(19) United States
(12) Patent Application Publication
(10) Pub. No.: US 2005/0272696 A1
(43) Pub. Date: Dec. 8, 2005

(54) Methods of selectively treating diseases with specific glycosaminoglycan polymers

(75) Inventor: Paul L. DeAngelis, Edmond, OK (US)

Correspondence Address: DUNLAP, CODDING & ROGERS P.C., PO BOX 16370, OKLAHOMA CITY, OK, 73113, US

(73) Assignee: Board of Regents of University of Oklahoma

(21) Appl. No.: 11/172,145

(22) Filed: Jun. 30, 2005

Related U.S. Application Data
(63) Continuation-in-part of application No. 10/642,248, filed on Aug. 15, 2003.
(63) Continuation-in-part of application No. 10/195,908, filed on Jul. 15, 2002.
Application No. 10/195,908 is a continuation-in-part of application No. 09/437,277, filed on Nov. 10, 1999, now Pat. No. 6,444,447.
Application No. 10/195,908 is a continuation-in-part of application No. 09/283,402, filed on Apr. 1, 1999, now abandoned.
Application No. 10/195,908 is a continuation-in-part of application No. 09/842,484, filed on Apr. 25, 2001, now abandoned.
Application No. 10/195,908 is a continuation-in-part of application No. 10/142,143, filed on May 8, 2002.
(60) Provisional application No. 60/584,442, filed on Jun. 30, 2004.
(60) Provisional application No. 60/404,356, filed on Aug. 16, 2002.
(60) Provisional application No. 60/479,432, filed on Jun. 18, 2003.
(60) Provisional application No. 60/491,362, filed on Jul. 31, 2003.
(60) Provisional application No. 60/107,929, filed on Nov. 11, 1998.
(60) Provisional application No. 60/080,414, filed on Apr. 2, 1998.
(60) Provisional application No. 60/199,538, filed on Apr. 25, 2000.
(60) Provisional application No. 60/289,554, filed on May 8, 2001.

(51) Int. Cl. A61K 31/728

(52) U.S. Cl. 514/54

(57) ABSTRACT

The present invention demonstrates that defined, specific GAG molecules have discerned differential effects, and that different types of cancers are prevented from proliferating and/or killed by oligosaccharides of different sizes; one size sugar does not treat all cancers effectively. Likewise, certain size GAGs have more potent angiogenic properties; thus, mixtures of different sizes of GAG molecules are not optimal. Therefore, the present invention is directed to methods of “personalized medicine”, in which customized defined, specific GAG molecules are administered to a patient, wherein the defined, specific GAG molecules are chosen based on the specific ailment from which the patient is suffering and/or the response of in vitro testing of the ability of the defined, specific GAG molecules to treat, inhibit and/or prevent the ailment in a sample from the patient.

### CROSS-REFERENCE TO RELATED APPLICATIONS

This application claims benefit under 35 U.S.C. 119(e) of provisional application U.S. Ser. No. 60/584,442, filed Jun. 30, 2004, the contents of which are hereby expressly incorporated herein by reference in their entirety.

This application is also a continuation-in-part of U.S. Ser. No. 10/642,248, filed Aug. 15, 2003; which claims benefit under 35 U.S.C. 119(e) of provisional applications U.S. Ser. No. 60/404,356, filed Aug. 16, 2002; U.S. Ser. No. 60/479,432, filed Jun. 18, 2003; and U.S. Ser. No. 60/491,362, filed Jul. 31, 2003; the contents of each of which are expressly incorporated herein by reference in their entirety.

Said application U.S. Ser. No. 10/642,248 is also a continuation-in-part of copending U.S. Ser. No. 10/195,908, filed Jul. 15, 2002; which is a continuation-in-part of U.S. Ser. No. 09/437,277, filed Nov. 11, 1999, now U.S. Pat. No. 6,444,447, issued Sep. 3, 2002; which claims benefit under 35 U.S.C. 119(e) of U.S. Provisional No. 60/107,929, filed Nov. 11, 1998, the contents of both of which are expressly incorporated herein in their entirety by reference.

Said U.S. Ser. No. 10/195,908 is also a continuation-in-part of U.S. Ser. No. 09/283,402, filed Apr. 1, 1999, now abandoned; which claims benefit under 35 U.S.C. 119(e) of U.S. Provisional No. 60/080,414, filed Apr. 2, 1998, the contents of both of which are expressly incorporated herein in their entirety by reference.

Said U.S. Ser. No. 10/195,908 is also a continuation-in-part of U.S. Ser. No. 09/842,484, filed Apr. 25, 2001, now abandoned; which claims benefit under 35 U.S.C. 119(e) of U.S. Provisional No. 60/199,538, filed Apr. 25, 2000, the contents of both of which are expressly incorporated herein in their entirety by reference.

Said U.S. Ser. No. 10/195,908 is also a continuation-in-part of U.S. Ser. No. 10/142,143, filed May 8, 2002; which claims benefit under 35 U.S.C. 119(e) of U.S. Provisional No. 60/289,554, filed May 8, 2001, the contents of both of which are expressly incorporated herein in their entirety by reference.

### STATEMENT REGARDING FEDERALLY SPONSORED RESEARCH OR DEVELOPMENT

This application was supported in part by National Research Grant C2163601 from the National Science Foundation. The United States Government may have rights in and to this application by virtue of this funding.

### BACKGROUND

1. Field of the Invention

The present invention relates to methodology for the use of defined, specific glycosaminoglycan molecules in the treatment of specific diseases and conditions, wherein the defined, specific glycosaminoglycan molecules exhibit differential effects in treatment of different diseases and conditions.

2. Description of the Related Art

Polysaccharides are large carbohydrate molecules comprising from about 25 sugar units to thousands of sugar units. Oligosaccharides are smaller carbohydrate molecules comprising less than about 25 sugar units. Animals, plants, fungi and bacteria produce an enormous variety of polysaccharide structures that are involved in numerous important biological functions such as structural elements, energy storage, and cellular interaction mediation. Often, the polysaccharide's biological function is due to the interaction of the polysaccharide with proteins such as receptors and growth factors. The glycosaminoglycan class of polysaccharides and oligosaccharides, which includes heparin, chondroitin, dermatan, keratan, and hyaluronic acid, plays major roles in determining cellular behavior (e.g., migration, adhesion) as well as the rate of cell proliferation in mammals. These polysaccharides and oligosaccharides are, therefore, essential for the correct formation and maintenance of the organs of the human body.

Several species of pathogenic bacteria and fungi also take advantage of the polysaccharide's role in cellular communication. These pathogenic microbes form polysaccharide surface coatings or capsules that are identical or chemically similar to host molecules. For instance, Group A & C Streptococcus and Type A Pasteurella multocida produce authentic hyaluronic acid capsules, and other Pasteurella multocida (Type F and D) and pathogenic Escherichia coli (K4 and K5) are known to make capsules composed of polymers very similar to chondroitin and heparin. The pathogenic microbes form the polysaccharide surface coatings or capsules because such a coating is nonimmunogenic and protects the bacteria from host defenses, thereby providing the equivalent of molecular camouflage.

Enzymes alternatively called synthases, synthetases, or transferases, catalyze the polymerization of polysaccharides found in living organisms. Many of the known enzymes also polymerize activated sugar nucleotides. The most prevalent sugar donors contain UDP, but ADP, GDP, and CMP are also used depending on (1) the particular sugar to be transferred and (2) the organism. Many types of polysaccharides are found at, or outside of, the cell surface. Accordingly, most of the synthase activity is typically associated with either the plasma membrane on the cell periphery or the Golgi apparatus membranes that are involved in secretion. In general, these membrane-bound synthase proteins are difficult to manipulate by typical procedures, and only a few enzymes have been identified after biochemical purification.

A larger number of synthases have been cloned and sequenced at the nucleotide level using “reverse genetic” approaches in which the gene or the complementary DNA (cDNA) was obtained before the protein was characterized. Despite this sequence information, the molecular details concerning the three-dimensional native structures, the active sites, and the mechanisms of catalytic action of the polysaccharide synthases, in general, are very limited or absent.

Some of the current methods for designing and constructing carbohydrate polymers in vitro utilize: (i) difficult, multistep sugar chemistry, or (ii) reactions driven by transferase enzymes involved in biosynthesis, or (iii) reactions harnessing carbohydrate degrading enzymes catalyzing transglycosylation or hydrolysis. The latter two methods are often restricted by the specificity and the properties of the available naturally occurring enzymes. Many of these enzymes are neither particularly abundant nor stable but are almost always expensive. Overall, the procedures currently employed yield polymers containing between 2 and about 12 sugars. Unfortunately, many of the physical and biological properties of polysaccharides do not become apparent until the polymer contains 25, 100, or even thousands of monomers.

As stated above, polysaccharides are the most abundant biomaterials on earth, yet many of the molecular details of their biosynthesis and function are not clear. Hyaluronic acid or “HA” is a linear polysaccharide of the glycosaminoglycan class and is composed of up to thousands of β(1,4)GlcUA-β(1,3)GlcNAc repeats. In vertebrates, HA is a major structural element of the extracellular matrix and plays roles in adhesion and recognition. HA has a high negative charge density and numerous hydroxyl groups; therefore, the molecule assumes an extended and hydrated conformation in solution. The viscoelastic properties of cartilage and synovial fluid are, in part, the result of the physical properties of the HA polysaccharide. HA also interacts with proteins such as CD44, RHAMM, and fibrinogen, thereby influencing many natural processes such as, but not limited to, angiogenesis, cancer, cell motility, wound healing, and cell adhesion.

HA is also made by certain microbes that cause disease in humans and animals. Some bacterial pathogens, namely Gram-negative Pasteurella multocida Type A and Gram-positive Streptococcus Group A and C, produce an extracellular HA capsule which protects the microbes from host defenses such as phagocytosis. Mutant bacteria that do not produce HA capsules are 10²- and 10³-fold less virulent in comparison to the encapsulated strains. Furthermore, the Paramecium bursaria Chlorella virus (PBCV-1) directs the algal host cells to produce a HA surface coating early in infection.

The various HA synthases (“HAS”), the enzymes that polymerize HA, utilize UDP-GlcUA and UDP-GlcNAc sugar nucleotide precursors in the presence of a divalent Mn, Mg, or Co ion to polymerize long chains of HA. The HA chains can be quite large (n=10² to 10⁴). In particular, the HASs are membrane proteins localized to the lipid bilayer at the cell surface. During HA biosynthesis, the HA polymer is transported across the bilayer into the extracellular space. In all HASs, a single species of polypeptide catalyzes the transfer of two distinct sugars. In contrast, the vast majority of other known glycosyltransferases transfer only one monosaccharide.

Recombinant PmHAS, PmCS, PmHS1, and PmHS2 elongate exogenous functional oligosaccharide acceptors to form long or short polymers in vitro; thus far no other Class I HA synthase has displayed this capability. The directionality of synthesis was established definitively by testing the ability of PmHAS and PmCS and PmHS1 and PmHS2 to elongate defined oligosaccharide derivatives. The non-reducing end sugar addition allows the reducing end to be modified for other purposes; the addition of GAG chains to small molecules, polymers, or surfaces is thus readily performed. Analysis of the initial stages of synthesis demonstrated that PmHAS and PmCS and PmHS1 and PmHS2 added single monosaccharide units sequentially. Apparently the fidelity of the individual sugar transfer reactions is sufficient to generate the authentic repeating structure of HA or chondroitin or heparin. Therefore, simultaneous addition of disaccharide block units is not required as hypothesized in some recent models of polysaccharide biosynthesis. PmHAS and PmCS and PmHS1 and PmHS2 appear distinct from most other known HA and chondroitin and heparin synthases based on differences in sequence, topology in the membrane, and/or putative reaction mechanism.

As mentioned previously, PmHAS, the 972-residue membrane-associated hyaluronan synthase, catalyzes the transfer of both GlcNAc and GlcUA to form an HA polymer. In order to define the catalytic and membrane-associated domains, PmHAS and PmCS mutants have been analyzed. PmHAS¹⁻⁷⁰³ is a soluble, active HA synthase suggesting that the carboxyl-terminus is involved in membrane association of the native enzyme. PmHAS¹⁻⁶⁵⁰ is inactive as a HA synthase, but retains GlcNAc-transferase activity. Within the PmHAS sequence, there is a duplicated domain containing a short motif, DGS or Asp-Gly-Ser, that is conserved among many glycosyltransferases. Changing this aspartate in either domain to asparagine, glutamate, or lysine reduced the HA synthase activity to low levels. The mutants substituted at residue 196 possessed GlcUA-transferase activity while those substituted at residue 477 possessed GlcNAc-transferase activity. The Michaelis constants of the functional transferase activity of the various mutants, a measure of the apparent affinity of the enzymes for the precursors, were similar to wild-type values. Furthermore, mixing D196N and D477K mutant proteins in the same reaction allowed HA polymerization at levels similar to the wild-type enzyme. These results provide the first direct evidence that the synthase polypeptide utilizes two separate glycosyltransferase sites. Likewise, PmCS mutants were made and tested having the same functionality (except GalNAc transferase activity) and sequence similarity to the mutants created for PmHAS. The same concept applies to PmHS1 and PmHS2, but different mutations must be made to produce the α4GlcNAc and β4 GlcA transferase activities.

The size of the HA polysaccharide dictates its biological effect in many cellular and tissue systems based on many reports in the literature. However, no source of very defined, uniform HA polymers with sizes greater than 5 kDa is currently available. This situation is complicated by the observation that long and short HA polymers appear to have antagonistic or inverse effects on some biological systems. Therefore, HA preparations containing a mixture of both size populations may yield contradictory or paradoxical results. Thus, one of the objects of the present invention is to provide a method to produce HA with very narrow, substantially monodisperse size distributions that overcomes the disadvantages and defects of the prior art.

The disease cancer has many potential clinical presentations and variables due to a combination of factors, including but not limited to: (1) the wide variety of tissues/organs of origin; (2) the biochemical differences in mutation site or physiological perturbations; and/or (3) the differences in the genetic makeup of patients. Therefore, the severity and the treatment of the disease will also vary. With respect to the use of novel glycomedicines such as GAG oligosaccharides, it is expected that not all disease states will be equal. However, there is no facile way to predict the outcome or the efficacy of any particular therapeutic molecule short of empirical testing.

Previously, other investigators have reported that mixtures of HA oligosaccharides have anticancer effects (Zeng et al., 1998). However, the most active components, as well as any inactive or inhibitory components, were not identified; thus, these formulations are not optimal and are not directly useful for treatment of mammals and humans.

Rapid blood vessel growth into the newly formed bone tissue is of paramount importance (Mowlem, 1963; Boume, 1972). Absence of adequate nutrient nourishment of the cells residing at the interior of large scaffolds after been implanted to a bone defect site will result in the death of the implanted cells and consequently the severe decrease of the possibility of bone regeneration. Apart from providing nutrients, rapid vascularization of bone grafts assists in the recruitment of osteoprogenitor and osteoclastic cells from the host tissue that will initiate the bone regeneration and remodeling cascade. The degradation products of hyaluronic acid (HA), oligoHA, are also known to stimulate endothelial-cell proliferation and to promote neovascularization associated with angiogenesis (West et al., 1985; Slevin et al., 2002).

Partial degradation products of sodium hyaluronate produced by the action of testicular hyaluronidase induced an angiogenic response (formation of new blood vessels) on the chick chorioallantoic membrane. Neither macromolecular hyaluronate nor exhaustively digested material had any angiogenic potential. Fractionation of the digestion products established that the activity was restricted to hyaluronate fragments between 4 and 25 disaccharides in length (West et al., 1985).

A delayed revascularization model was used previously to assess the angiogenic activity of hyaluronan fragments on impaired wound healing (Lees et al., 1995). 1- to 4-kDa hyaluronan fragments increased blood flow and increased graft vessel growth, whereas 33-kDa fragments had no such effect on graft blood flow or vessel growth.

Different cells in different tissues have different signalling pathways (due to varied levels and/or components that make each cell type distinct); thus, the effect of HA and oligosaccharides cannot be predicted. Empirical testing for each tissue is thus indicated. In addition, prior to the present invention, there was not a reliable supply of individual nanoHA sizes for investigating their effects.

Parent application U.S. Ser. No. 10/642,248, filed Aug. 15, 2003, the contents of which have been previously incorporated herein by reference, discloses and claims methods for the production of glycosaminoglycans of HA, chondroitin, and chimeric or hybrid molecules incorporating both HA and chondroitin, wherein the glycosaminoglycans are substantially monodisperse and thus have a defined size distribution.

The present invention discloses studies with the defined, specific GAG molecules disclosed and claimed in U.S. Ser. No. 10/642,248, and the presently disclosed and claimed invention demonstrates that these defined, specific GAG molecules have discerned differential effects. Briefly, the presently disclosed and claimed invention demonstrates that different types of cancers are prevented from proliferating and/or killed (or induced to undergo programmed suicide or apoptosis) by oligosaccharides of different sizes; one size sugar does not treat all cancers effectively. Likewise, the effects of GAG molecules on vascularization and angiogenesis are also size dependent. Therefore, the presently disclosed and claimed invention is directed to methods of “personalized medicine”, in which customized defined, specific GAG molecules are administered to a patient, wherein the defined, specific GAG molecules are chosen based on the specific ailment from which the patient is suffering and/or the response of in vitro testing of the ability of the defined, specific GAG molecules to treat, inhibit and/or prevent the ailment in a sample (i.e., biopsy) from the patient.

### SUMMARY OF THE INVENTION

The present invention is related to a method of inhibiting or preventing a disease or condition in a patient. The method includes identifying a disease or condition in a patient, such as cancer or a disease associated with abnormal levels of angiogenesis, and selecting a glycosaminoglycan polymer having a specific size distribution, wherein the glycosaminoglycan polymer having the specific size distribution is effective in inhibiting the disease or condition. A composition is then provided which comprises recombinantly-produced defined glycosaminoglycan polymers having the desired specific size distribution such that the glycosaminoglycan polymers are substantially monodisperse in size, wherein at least 95% of the composition comprises the defined glycosaminoglycan polymers having the desired specific size distribution and less than 5% of the composition comprises glycosaminoglycan polymers of a different size distribution. The composition is then administered to the patient in an amount effective to inhibit the disease or condition.

In one embodiment, the desired size distribution may be obtained by controlling a stoichiometric ratio of UDP-sugar to functional acceptor in the recombinant production thereof.

The substantially monodisperse glycosaminoglycan polymers may have a molecular weight in a range of from about 600 Da to about 3.5 kDa and a polydispersity value in a range of from about 1.0 to about 1.1, such as in a range of from about 1.0 to about 1.05. The defined glycosaminoglycan polymers may be defined hyaluronan polymers having a size distribution in a range of from HA10 to HA25, such as HA10, HA12, HA20 or HA22. Optionally, the glycosaminoglycan polymers may be chimeric or hybrid glycosaminoglycans having a non-natural structure.

Optionally, when the desired size distribution is obtained by controlling a stoichiometric ratio of UDP-sugar to functional acceptor in the recombinant production thereof, the substantially monodisperse glycosaminoglycan polymers may have a molecular weight in a range of from about 3.5 kDa to about 0.5 MDa, or a molecular weight in a range of from about 0.5 MDa to about 4.5 Mda. The substantially monodisperse glycosaminoglycan polymers may have a polydispersity value in a range of from about 1.0 to about 1.1, such as a range of from about 1.0 to about 1.05.

In one embodiment, the disease or condition is a first type of cancer, and the desired size distribution of the glycosaminoglycan polymer is effective in inhibiting the first type of cancer, but is not effective in inhibiting a second type of cancer.

The defined glycosaminoglycan polymer may be produced by a method that includes providing at least one functional acceptor, wherein the functional acceptor has at least two sugar units selected from the group consisting of uronic acid, hexosamine, structural variants and derivatives thereof, a hyaluronan polymer, a chondroitin polymer, a chondroitin sulfate polymer, a heparosan-like polymer, a heparinoid, mixed GAG chains, analog containing chains, and combinations thereof, providing at least one recombinant glycosaminoglycan transferase capable of elongating the at least one functional acceptor in at least one of a controlled fashion and a repetitive fashion to form extended glycosaminoglycan-like molecules, and providing at least one UDP-sugar selected from the group consisting of UDP-GlcUA, UDP-GlcNAc, UDP-Glc, UDP-GalNAc, UDP-GlcN, UDP-GalN and structural variants or derivatives thereof in a stoichiometric ratio to the at least one functional acceptor such that the at least one recombinant glycosaminoglycan transferase elongates the at least one functional acceptor to provide glycosaminoglycan polymers wherein the glycosaminoglycan polymers have a desired size distribution such that the glycosaminoglycan polymers are substantially monodisperse in size.

In the method described above, uronic acid may further be defined as a uronic acid selected from the group consisting of GlcUA, IdoUA, GalUA, and structural variants or derivatives thereof, and hexosamine may further be defined as a hexosamine selected from the group consisting of GlcNAc, GalNAc, GlcN, GalN, and structural variants or derivatives thereof. The at least one functional acceptor may be selected from the group consisting of a chondroitin oligosaccharide comprising at least about three sugar units, a chondroitin polymer, a chondroitin sulfate polymer, a heparosan-like polymer, a heparinoid, and an extended acceptor selected from the group consisting of HA chains, chondroitin chains, heparosan chains, mixed glycosaminoglycan chains, analog containing chains, a sulfated functional acceptor, a modified oligosaccharide, and combinations thereof. The at least one recombinant glycosaminoglycan transferase may be selected from the group consisting of a recombinant hyaluronan synthase or active fragment or mutant thereof; a recombinant chondroitin synthase or active fragment or mutant thereof; a recombinant heparosan synthase or active fragment or mutant thereof; a recombinant single action glycosyltransferase capable of adding only one of GlcUA, GlcNAc, GlcN, GalNAc, GlcN, GalN or a structural variant or derivative thereof; a recombinant synthetic chimeric glycosaminoglycan transferase capable of adding two or more of GlcUA, GlcNAc, GlcN, GalNAc, GlcN, GalN or a structural variant or derivative thereof; and combinations thereof. The method may further comprise at least one of: (A) the at least one functional acceptor is a plurality of functional acceptors immobilized on a substrate; (B) the at least one functional acceptor is a plurality of functional acceptors in a liquid phase; (C) the at least one recombinant glycosaminoglycan transferase is immobilized and the at least one functional acceptor and the at least one of UDP-GlcUA, UDP-GlcNAc, UDP-Glc, UDP-GalNAc, UDP-GlcN, UDP-GalN and a structural variant or derivative thereof are in a liquid phase; and (D) the at least one functional acceptor is immobilized and the at least one UDP-sugar are in a liquid phase.

The method may further include the step of providing a divalent metal ion, wherein the divalent metal ion is selected from the group consisting of manganese, magnesium, cobalt, nickel and combinations thereof, and the method may occur in a buffer having a pH from about 6 to about 8. The at least one recombinant glycosaminoglycan transferase may be selected from the group consisting of: (A) a recombinant glycosaminoglycan transferase having an amino acid sequence encoded by a nucleotide sequence capable of hybridizing under standard stringent, moderately stringent, or less stringent hybridization conditions to a nucleotide sequence selected from the group consisting of SEQ ID NOS:1, 3, 5, 7, 9 or 11; (B) a recombinant glycosaminoglycan transferase having an amino acid sequence essentially as set forth in SEQ ID NO:2, 4, 6, 8, 10, 12-22 or 25; (C) a recombinant glycosaminoglycan transferase encoded by a nucleotide sequence essentially as set forth in SEQ ID NO:1, 3, 5, 7, 9 or 11; and (D) a recombinant glycosaminoglycan transferase having at least one motif selected from the group consisting of SEQ ID NOS:23 and 24. The at least one functional acceptor may comprise a moiety selected from the group consisting of a fluorescent tag, a radioactive tag, an affinity tag, a detection probe, a medicant, and combinations thereof. Optionally, the at least one UDP-sugar may be radioactively labeled.

The present invention is also directed to a kit that includes at least two compositions comprising recombinantly-produced defined glycosaminoglycan polymers having desired specific size distributions such that the glycosaminoglycan polymers of each composition are substantially monodisperse in size, as described herein above. The kit also includes means for testing the ability of each of the defined glycosaminoglycan polymers to inhibit or prevent a disease or condition (such as cancer or a disease or condition associated with abnormal levels of angiogenesis) in a sample from a patient, such as a biopsy. One desired size distribution of the glycosaminoglycan polymer may be effective in inhibiting or preventing the disease or condition, while a different size distribution of the glycosaminoglycan polymer is not effective in inhibiting or preventing the disease or condition. The kit may be a catalog available on the World Wide Web.

The present invention is also related to a method of inhibiting or preventing a disease or condition in a patient that includes providing at least two compositions comprising recombinantly-produced defined glycosaminoglycan polymers having desired specific size distributions such that the glycosaminoglycan polymers of each composition are substantially monodisperse in size, as described herein above. A sample (such as a biopsy) from a patient suffering from or predisposed for a disease or condition is provided, and each of the at least two defined glycosaminoglycan polymer compositions is reacted with a portion of the sample from the patient. At least one defined glycosaminoglycan polymer composition that inhibits or prevents the disease or condition in the sample is identified, and the patient is administered an effective amount of the defined glycosaminoglycan polymer composition that inhibited or prevented the disease or condition in the sample, thus inhibiting or preventing the disease or condition in the patient. One desired size distribution of the glycosaminoglycan polymer may be effective in inhibiting or preventing the disease or condition, while a different size distribution of the glycosaminoglycan polymer is not effective in inhibiting or preventing the disease or condition.

Other objects, features and advantages of the present invention will become apparent from the following detailed description when read in conjunction with the accompanying drawings and appended claims.

### BRIEF DESCRIPTION OF THE SEVERAL VIEWS OF THE DRAWINGS

FIG. 1 is a graphical representation of a hypothetical model of HA effects on cancer.

FIG. 2 is a graphical representation of a schematic comparing the methods of the present invention to prior art methods for HA oligosaccharide synthesis.

FIG. 3 is a graphical representation of a schematic comparing the methods of the present invention to prior art methods of novel sugar syntheses.

FIG. 4 is a graphical representation illustrating elongation of sugar acceptor by pmHAS. This thin layer chromatogram depicts the sugar HA4 (GlcNAc-GlcUA-GlcNAc-GlcUA; see +0 control lane) being elongated by one sugar when UDP-GlcNAc was in the reaction (see +N). No change is seen if the UDP-GlcUA (lane +A) is present as GlcUA is not added until the next step of synthesis. When both UDP-sugars are present (lane +AN), extension of HA4 into HA7, 9, 11, 13 is observed. (Lane s, HA sugar standards; arrow marks the origin).

FIG. 5 is a graphical representation of pmHAS structure. Two relatively independent active sites exist in one polypeptide. Specific mutations are utilized to molecularly dissect a dual-action enzyme into two single-action enzymes suitable for use in bioreactors.

FIG. 6 is an electrophoresis gel illustrating isolation of pmHAS¹⁻⁷⁰³. This Coomassie-stained, SDS-polyacrylamide gel was used to monitor the purification of the soluble, dual-action pmHAS produced in recombinant Escherichia coli bacteria. After two chromatographic steps (ion exchange, IE; gel filtration, GF), the catalyst is 90-95% pure and fully functional (arrow). Similar preparations of the single-action mutants are suitable for generating a bioreactor.

FIG. 7 is a mass spectra analysis of the F-HA12 product. A fluorescent HA12 oligosaccharide was synthesized using a twin reactor scheme as described herein. A peak with the predicted mass is apparent; no shorter HA11 sugar or longer HA13 sugar is observed.

FIG. 8 is a graphical representation of a microarray library of variants—overview of drug discovery process.

FIG. 9 is a graphical representation of the biocatalytic scheme of the present invention, including a step-wise addition of sugars.

FIG. 10 is a gel analysis of in vitro synchronized, liquid-phase HA synthesis products in the presence or absence of HA4 acceptor. A matched set of reactions (100 μl each) containing 12 μM pmHAS, 30 mM UDP-GlcNAc, 30 mM UDP-GlcUA and either 38 μM HA4 acceptor (+) OR no acceptor (−) was incubated for 48 hours. A portion (0.2 μl) of the reactions was analyzed on a 0.7% agarose gel and Stains-All detection. For comparison, DNA standards were run (D, Bioline DNA HyperLadder, top to bottom —10, 8, 6, 5, 4, 3, 2.5, 2, 1.5, 1, 0.8, 0.5 kb; D′, Invitrogen high-MW DNA ladder, top band 48.5 kb). A smaller, narrow size distribution HA polymer is formed by pmHAS in presence of HA4 as seen by the faster migrating, tight gel band.

FIG. 11 is a SEC-MALLS analysis of in vitro HA synthesis products in the presence or absence of HA4 acceptor. The refractometer concentration peaks (lines) and the molar mass curves (symbols with corresponding y-axis scale) of the matched set of reactions described in FIG. 1 are shown on the same PL aquagel-OH 60 size exclusion chromatography (SEC) column profile. A smaller, narrow size distribution HA polymer is formed by pmHAS in the presence of HA4 (thick line and squares) as evidenced by its later elution time and flatter molar mass curve (generated by multi-angle laser light scattering) in comparison to the reaction without acceptor (thin line and circles).

FIG. 12 are electrophoresis gels illustrating intermediate size HA polysaccharides as acceptors. The starting 20 μl reaction contain 15 μg of pmHAS, 10 mM UDP-sugars and 5 μg HA4. 5 μl of 40 mM UDP-sugars and 15 μg of pmHAS were supplied additionally every 48 hours (“feeding”). A. 1% agarose gel electrophoresis. Lane 1, 3 feedings. Lane 2, 2 feedings. Lane 3, one feedings. Lane 4, no feeding. D1, Bio-Rad 1 kb DNA ruler. D2 Lambda HindIII DNA. D3, Bio-Rad 100 bp DNA ruler. B. 15% acrylamide gel electrophoresis. Lane 1-4, same as in panel A.

FIG. 13 is a graphical representation of schematic models for acceptor-mediated synchronization and polymer size control. Panel A depicts the reaction in vitro where UDP-sugars (black triangle UDP; small black or white ovals, monosaccharides) are bound to the pmHAS (HAS) and the first glycosidic linkages are formed over a lag period due to this rate-limiting step (slow initiation). Once the initial HA chain is started, then subsequent sugars are added rapidly to the nascent polymer (fast elongation) by the enzyme. It is probable that some chains are initiated before other chains (short lag versus long lag period, respectively); thus, asynchronous polymerization occurs, resulting in a population of HA product molecules with a broad size distribution. Panel B depicts the reaction where the acceptor sugar (striped bar) bypasses the slow initiation step. Thus, all chains are elongated by the nonprocessive pmHAS in a parallel, synchronous fashion resulting in a uniform HA product with a narrow size distribution. Panel C illustrates that if a large amount of acceptor molecules and a finite amount of UDP-sugars are present, then the UDP-sugars are distributed among the acceptors to produce shorter polymers than in the case with a smaller quantity of acceptors (resulting in longer chain extensions as shown in Panel 13B). Therefore, it is possible to adjust the molar ratio of acceptor to UDP-sugars to control the ultimate polymer molecular mass.

FIG. 14 is a graphic representation of control of HA product size by adjusting acceptor/UDP-sugar ratio. Decreasing amounts of acceptor sugar (lanes 1-5: final concentration=50, 38, 30, 25, or 19 μM HA4) were added to reactions (100 μl, 72 hours) containing 8 μM pmHAS, 32 mM of UDP-GlcNAc, 32 mM of UDP-GlcUA. Purified synthetic HA (1 μg) was analyzed on a 1.2% agarose gel and Stains-All. The average molecular masses and polydispersity of HA were also determined by SEC-MALLS (Mw and Mw/Mn for lane 1, 284 kDa: 1.001; 2, 347 kDa: 1.002; 3, 424 kDa: 1.004; 4, 493 kDa: 1.006; 5, 575 kDa: 1.01). The position of certain DNA standards is marked (kb). The use of higher acceptor/UDP-sugar ratios results in shorter HA chains.

FIG. 15 is a graphic representation of comparison of synthetic HA versus natural HA preparations. A variety of HA samples either synthesized by synchronized chemoenzymatic reactions in vitro or derived from streptococcal bacteria or chicken sources were analyzed on a 0.7% agarose gel with Stains-All detection. The Mw of each synthetic HA polymer was determined by SEC-MALLS. Lane 1, a mixture of synthetic HA polymers produced in five different reactions, bottom to top, 27, 110, 214, 310 and 495 kDa; 2, a mixture of HA polymers produced in five different reactions, bottom to top, 495, 572, 966, 1090 and 1510 kDa; 3, 2.0 MDa synthetic HA; 4, rooster comb HA (Sigma); 5, streptococcal HA (Sigma); 6-7, streptococcal HA (Lifecore); D, DNA HyperLadder. The tight bands of the synthetic HA polymers indicate their relative monodispersity in comparison to extracted HA.

FIG. 16 is a graphic representation of synthesis of various monodisperse fluorescent-end labeled HA polymers (suitable as probes). A series of parallel reactions (20 μl, 72 hours) containing 24 μM pmHAS, 34 μM fluor-HA4 and decreasing amounts of UDP-GlcNAc and UDP-GlcUA (lanes 1-4: final concentration=32, 25, 20 or 15 mM each) were prepared. Portions of the reactions (1 μl) were analyzed on a 0.7% agarose gel. The signal of the fluorescent tag was detected with long wave UV excitation. The position of certain DNA standards is marked (kb). The use of higher acceptor/UDP-sugar molar ratios results in shorter HA chains. A drug or medicament can be similarly added to GAG chains.

FIG. 17 is an electrophoresis gel illustrating utilization of large HA acceptors. Reactions were carried out at 30° C. for 48 hours. The 60 μl reaction contained 0.28 μg/μl of pmHAS, 3.3 mM UDP-GlcNAc, 3.3 mM UDP-GlcUA and without (lane 2) or with various amounts of acceptors (lanes 3-5, 7-9 and 10). 1.0 μl of each reaction was loaded on 0.7% agarose gel and stained with STAINS-ALL. Lane 1, BIORAD kb ladder (top band is 15 kb). Lane 6, 0.5 μg of 970 kDa HA starting acceptor. Lane 11, 3 μg of Genzyme HA starting acceptor. Lane 12, Invitrogen DNA HyperLadder (top band is 48.5 kB).

FIG. 18 is an electrophoresis gel that illustrates the migration of a ladder constructed of HA of defined size distribution for use as a standard.

FIG. 19 is an electrophoresis gel illustrating various mondisperse chondroitin sulfate HA hybrid GAGs. The 1% agarose gel stained with STAINS-ALL shows a variety of chondroitin sulfates (either A, B or C) that were elongated with pmHAS, thus adding HA chains. Lanes 1, 8, 15, 22 and 27 contain the Kilobase DNA ladder; lanes 2 and 7 contain starting CSA, while lanes 3-6 contain CSA-HA at 2 hrs, 4 hrs, 6 hrs and O/N, respectively; lanes 9 and 14 contain starting CSB, while lanes 10-13 contain CSB-HA at 2 hrs, 4 hrs, 6 hrs and O/N, respectively; lanes 16 and 21 contain starting CSC, while lanes 17-20 contain CSC-HA at 2 hrs, 4 hrs, 6 hrs and O/N, respectively; lanes 23-26 contain no acceptor at 2 hrs, 4 hrs, 6 hrs and O/N, respectively.

FIG. 20 is an electrophoresis gel illustrating control of hybrid GAG size by stoichiometric control. The 1% agarose gel stained with STAINS-ALL shows chondroitin sulfate A that was elongated with pmHAS, thus adding HA chains. Lanes 1, 7, 13, 19 and 25 contain the Kilobase ladder; lanes 2 and 6 contain 225 μg starting CSA, while lanes 3-5 contain 225 μg CSA-HA at 2 hrs, 6 hrs and O/N, respectively; lanes 8 and 12 contain 75 μg starting CSA, while lanes 9-11 contain 75 μg CSA-HA at 2 hrs, 6 hrs and O/N, respectively; lanes 14 and 18 contain 25 μg starting CSA, while lanes 15-17 contain 25 μg CSA-HA at 2 hrs, 6 hrs and O/N, respectively; lanes 20 and 24 contain 8.3 μg starting CSA, while lanes 21-23 contain 8.3 μg CSA-HA at 2 hrs, 6 hrs and O/N, respectively.

FIG. 21 is an electrophoresis gel illustrating extension of HA with chondroitin chains using pmCS. The 1.2% agarose gel stained with STAINS-ALL shows a reaction with pmCS and UDP-GlcUA, UDP-GalNAc with either a 81 kDa HA acceptor (lanes 3-7) or no acceptor (lanes 9-13). Lanes 1 and 15 contain the Kilobase DNA standard. Lanes 2, 8 and 14 contain starting 81 kDa HA. Lanes 3-7: contain HA acceptor +HA-C at 2 hr, 4 hr, 4 hr (set O/N in incubator without 4 hr feeding), 6 hr and O/N, respectively. Lanes 9-13: contain no acceptor (minus)-HA-C at 2 hr, 4 hr, 4 hr (set O/N in incubator without 4 hr feeding), 6 hr and O/N, respectively.

FIG. 22 is a size exclusion (or gel filtration) chromatography analysis coupled with multi-angle laser light scattering detection (SEC-MALLS) confirms the monodisperse nature of polymers created by the present invention. In A, HA (starting MW 81 kDa) extended with chondroitin chains using pmCS (same sample used in FIG. 21 lane #7, overnight [O/N] extension) was analyzed; the material was 280,000 Mw and polydispersity (Mw/Mn) was 1.003+/−0.024. Chondroitin sulfate extended with HA chains using pmHAS (same sample used in FIG. 31, lane #23) was analyzed and shown in the bottom chromatogram; the material was 427,000 Mw and polydispersity (Mw/Mn) was 1.006+/−0.024.

FIG. 23 is an 0.7% agarose gel detected with Stains-all compares the monodisperse, ‘select HA’ to commercially produced HA samples.

FIG. 24 is a schematic of catalyst generation and dual-enzyme reactor scheme. Panel A. Mutagenesis was used to transform the dual-action HA synthase into two single-action catalysts (GN-T, GlcNAc-transferase; GA-T, GlcUA-transferase). The resulting enzymes were purified and immobilized onto agarose beads. Panel B. A starting acceptor (e.g., tetrasaccharide HA4) is combined with the UDP-GlcNAc precursor and circulated through the GN-T reactor (GlcNAc, open circle; GlcUA, solid circle). After coupling, UDP-GlcUA precursor is added to the mixture and circulated through the GA-T reactor. This stepwise synthesis is repeated as desired (dashed line) until the target oligosaccharide size is reached. In this study, a total of 16 addition steps were performed to produce HA20.

FIG. 25 is a gel electrophoretic analysis of HA20 Synthesis. Samples of the crude reaction mixture from the sequential sugar addition steps were analyzed on a polyacrylamide gel. No runaway polymerization is observed even though both UDP-sugar precursors were present at high concentration throughout the synthesis. Note that even-numbered oligosaccharides with a higher charge to mass ratio migrate faster than odd-numbered oligosaccharides in this system. (S=ladder of native HA digested with hyaluronidase).

FIG. 26 is a mass spectra of HA oligosaccharides. MALDI-TOF MS was performed on the desalted HA oligosaccharides from three independent preparations synthesized with the pair of enzyme reactors. The target polymers have the appropriate molecular mass (expected isotopic mass/experimental mass: HA13, 2494.75/2494.94 Da; HA14, 2670.78/2670.92 Da; HA20, 3808.18/3808.58 Da) and are the major components.

FIG. 27 is a graphic representation of the results of a standard soft agar growth test of the drug-resistant human uterine sarcoma cell line MES-SA/Dx5 in the presence of Paclitaxel (a positive control chemotherapy agent; 1 μg/ml) or nanoHA (HA4, 10, 12, 14, 22; 100 μg/ml). Water (H₂O) is used as a negative control. HA12 is the most effective of the tested nanoHAs for this type of cancer.

FIG. 28 is a graphic representation of the results of a standard soft agar growth test of the human colon adenocarcinoma cell line HCT-116 in the presence of Paclitaxel (1 μg/ml) or nanoHA (HA4, 10, 12, 14, 22; 100 μg/ml). HA22 is the most effective of tested nanoHAs for this type of cancer.

FIG. 29 is a graphic representation demonstrating the angiogenic capacity of nanoHA (HA4, 8, 12, 18, 20 and 22) as determined by increased number of blood vessels in the avian chorioallantoic membrane (CAM) egg assay. In this assay, HA20 is the most effective of the tested nanoHAs.

FIG. 30 is a graphic representation demonstrating the angiogenic capacity of nanoHA (HA4, 8, 12, 18, 20 and 22) as determined by enhanced fractional image area of blood vessels (higher area is more angiogenesis) in the CAM assay. In this assay, HA20 is the most effective of the tested nanoHAs.

### DETAILED DESCRIPTION OF THE INVENTION

Before explaining at least one embodiment of the invention in detail, it is to be understood that the invention is not limited in its application to the details of construction and the arrangements of the components set forth in the following description or illustrated in the drawings. The invention is capable of other embodiments or of being practiced or carried out in various ways. Also, it is to be understood that the phraseology and terminology employed herein is for purpose of description and should not be regarded as limiting.

Glycosaminoglycans (“GAGs”) are linear polysaccharides composed of repeating disaccharide units containing a derivative of an amino sugar (either glucosamine or galactosamine). Hyaluronan [HA], chondroitin, and heparan sulfate/heparin contain a uronic acid as the other component of the disaccharide repeat while keratan contains a galactose. The GAGs are summarized in Table I.

TABLE I

| | | Post-Polymerization | |
|---|---|---|---|
| | Disaccharide | Modifications | |
| Polymer | Repeat | Vertebrates | Bacteria |
| Hyaluronan | β3GlcNAc β4GlcUA | none | none |
| Chondroitin | β3GalNAc β4GlcUA | O-sulfated/ | none |
| | | epimerized | |
| Heparin/heparan | α4GlcNAc β4GlcUA | O,N-sulfated/ | none |
| | | epimerized | |
| Keratan | β4GlcNAc β3Gal | O-sulfated | not reported |

GAGs and their derivatives are currently used in the medical field as ophthalmic and viscoelastic supplements, adhesion surgical aids to prevent post-operative adhesions, catheter and device coatings, and anticoagulants. Other current or promising future applications include anti-cancer medications, tissue engineering matrices, immune and neural cell modulators, and drug targeting agents.

Complex carbohydrates, such as GAGs, are information rich molecules. A major purpose of the sugars that make up GAGs is to allow communication between cells and extracellular components of multicellular organisms. Typically, certain proteins bind to particular sugar chains in a very selective fashion. A protein may simply adhere to the sugar, but quite often the protein's intrinsic activity may be altered and/or the protein transmits a signal to the cell to modulate its behavior. For example, in the blood coagulation cascade, heparin binding to inhibitory proteins helps shuts down the clotting response. In another case, HA binds to cells via the CD44 receptor that stimulates the cells to migrate and to proliferate. Even though long GAG polymers (i.e., >10² Da) are found naturally in the body, typically the protein's binding site interacts with a stretch of 4 to 10 monosaccharides. Therefore, oligosaccharides can be used to either (a) substitute for the polymer or (b) to inhibit the polymer's action depending on the particular system.

HA polysaccharide plays structural roles in the eye, skin, and joint synovium. Large HA polymers (˜10⁶ Da) also stimulate cell motility and proliferation. On the other hand, shorter HA polymers (˜10⁴ Da) often have the opposite effect. HA-oligosaccharides composed of about 10 to 25 sugars [HA₁₀₋₂₅] have promise for inhibition of cancer cell growth and metastasis. For example, in an in vivo assay, mice injected with various invasive and virulent tumor cell lines (melanoma, glioma, carcinomas from lung, breast and ovary) develop a number of large tumors and die within weeks. Treatment with HA oligosaccharides greatly reduced the number and the size of tumors (Zeng et al., 1998). Metastasis, the escape of cancer cells throughout the body, is one of the biggest fears of both the ailing patient and the physician. HA or HA-like oligosaccharides appear to serve as a supplemental treatment to inhibit cancer growth and metatasis.

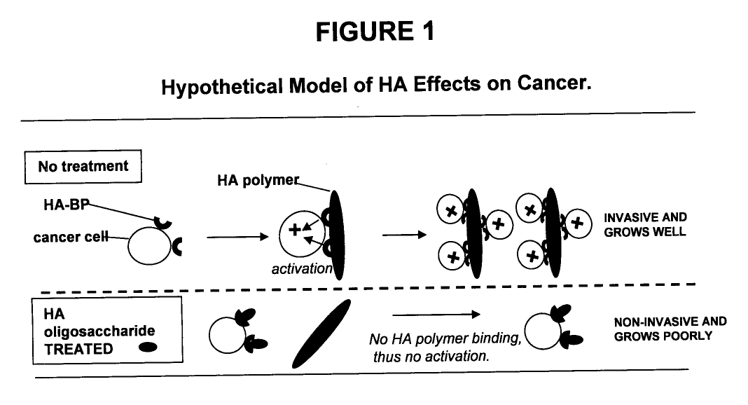

The preliminary mode of action of the HA-oligosaccharide sugars is thought to be mediated by binding or interacting with one of several important HA-binding proteins (probably CD44 or RHAM) in the mammalian body. One proposed scenario for the anticancer action of HA-oligosaccharides is that multiple CD44 protein molecules in a cancer cell can bind simultaneously to a long HA polymer (FIG. 1). This multivalent HA binding causes CD44 activation (perhaps mediated by dimerization or a receptor patching event) that triggers cancer cell activation and migration. However, if the cancer cell is flooded with small HA-oligosaccharides, then each CD44 molecule individually binds a different HA molecule in a monovalent manner such that no dimerization/patching event occurs. Thus no activation signal is transmitted to the cell (FIG. 1). The prior art believed that the optimal HA-sugar size was 10 to 14 sugars. Although this size may be based more upon the size of HA currently available for testing rather than biological functionality—i.e., now that HA molecules and HA-like derivatives <10 sugars are available according to the methodologies of the present invention, the optimal HA size or oligosaccharide composition may be found to be different.

It has also been shown that treatment with certain anti-CD44 antibodies or CD44-antisense nucleic acid prevents the growth and metastasis of cancer cells in a fashion similar to HA-oligosaccharides; in comparison to the sugars, however, these protein-based and nucleic acid-based reagents are somewhat difficult to deliver in the body and/or may have long-term negative effects. A very desirable attribute of HA-oligosaccharides for therapeutics is that these sugar molecules are natural by-products that can occur in small amounts in the healthy human body during the degradation of HA polymer; no untoward innate toxicity, antigenicity, or allergenic concerns are obvious.

Other emerging areas for the potential therapeutic use of HA oligosaccharides are the stimulation of blood vessel formation and the stimulation of dendritic cell maturation. Enhancement of wound-healing and resupplying cardiac oxygenation may be additional applications that harness the ability of HA oligosaccharides to cause endothelial cells to form tubes and sprout new vessels. Dendritic cells possess adjuvant activity in stimulating specific CD4 and CD8 T cell responses. Therefore, dendritic cells are targets in vaccine development strategies for the prevention and treatment of infections, allograft reactions, allergic and autoimmune diseases, and cancer.

Heparin interacts with many proteins in the body, but two extremely interesting classes are coagulation cascade proteins and growth factors. Antithrombin III [ATIII] and certain other hemostasis proteins are 100,000-fold more potent inhibitors of blood clotting when complexed with heparin. Indeed, heparin is so potent it must be used in a hospital setting and require careful monitoring in order to avoid hemorrhage. Newer, processed lower molecular weight forms of heparin are safer, but this material is still a complex mixture. It has been shown that a particular pentasaccharide (5 sugars long) found in heparin is responsible for the ATIII-anticoagulant effect. But since heparin is a very heterogeneous polymer, it is difficult to isolate the pentasaccharide (5 sugars long) in a pure state. The pentasaccharide can also be prepared in a conventional chemical synthesis involving ˜50 to 60 steps. However, altering the synthesis or preparing an assortment of analogs in parallel is not always feasible—either chemically or financially.

Many growth factors, including VEGF (vascular endothelial growth factor), HBEGF (heparin-binding epidermal growth factor), and FGF (fibroblast growth factor), bind to cells by interacting simultaneously with the growth factor receptor and a cell-surface heparin proteoglycan; without the heparin moiety, the potency of the growth factor plummets. Cell proliferation is modulated in part by heparin; therefore, diseases such as cancer and atherosclerosis are potential targets. Abnormal or unwanted proliferation would be curtailed if the growth factor was prevented from stimulating target disease-state cells by interacting with a heparin-like oligosaccharide analog instead of a surface-bound receptor. Alternatively, in certain cases, the heparin oligosaccharides alone have been shown to have stimulatory effects.

Chondroitin is the most abundant GAG in the human body, but all of its specific biological roles are not yet clear. Phenomenon such as neural cell outgrowth appear to be modulated by chondroitin. Both stimulatory and inhibitory effects have been noted depending on the chondroitin form and the cell type. Therefore, chondroitin or similar molecules are of utility in re-wiring synaptic connections after degenerative diseases (e.g., Alzheimer's) or paralytic trauma. The epimerized form of chondroitin (GlcUA converted to the C5 isomer, iduronic acid or IdoUA), dermatan, selectively inhibits certain coagulation proteins such as heparin cofactor II. By modulating this protein in the coagulation pathway instead of ATIII, dermatan appears to allow for a larger safety margin than heparin treatment for reduction of thrombi or clots that provoke strokes and heart attacks.

Many details of GAG/protein interactions are not yet clear due to (a) the heterogeneity of GAGs (in part due to their biosynthesis pathway) and (b) the difficulty in analyzing long polysaccharides and membrane receptor proteins at the molecular level. Fortunately, many short oligosaccharides have biological activities that serve to assist research pursuits as well as to treat disease in the near future. Conventional chemical synthesis of short GAG oligosaccharides is possible, but the list of roadblocks includes: (i) difficult multi-step syntheses that employ toxic catalysts, (ii) very low yield or high failure rates with products longer than ˜6 monosaccharides, (iii) imperfect control of stereoselectivity (e.g., wrong anomer) and regioselectivity (e.g., wrong attachment site), and (iv) the possibility for residual protection groups (non-carbohydrate moieties) in the final product.

It is well established that the large array of functions that a tumor cell has to fulfill to settle as a metastasis in a distant organ requires cooperative activities between the tumor and the surrounding tissue and that several classes of molecules are involved, such as cell-cell and cell-matrix adhesion molecules and matrix degrading enzymes, to name only a few. Furthermore, metastasis formation requires concerted activities between tumor cells and surrounding cells as well as matrix elements and possibly concerted activities between individual molecules of the tumour cell itself. CD44 transmembrane glycoproteins belong to the families of adhesion molecules and have originally been described to mediate lymphocyte homing to peripheral lymphoid tissues. It was soon recognized that the molecules, under selective conditions, may suffice to initiate metastatic spread of tumor cells (Marhaba et al., 2004). CD44 variant isoforms have been implicated in many biological processes, such as cell adhesion, cell substrate, cell to cell interactions, including lymphocyte homing haemopoiesis, cell migration and metastasis. These abilities are of great importance in chronic inflammation and in cancer. CD44 has shown the ability to recruit leucocytes to vascular endothelium at sites of inflammation, which is one of the first steps in the inflammatory response. In cancer, deregulation of the adhesion mechanisms increases the ability of tumor cells to metastasis. This behavior seems to be explained by the existing relationship between hyaluronan and CD44, which is its major cell surface receptor. There are CD44 variant isoforms (i.e., similar, but not functionally equivalent) which are expressed on different types of normal cells. In addition some isoforms are overexpressed on tumor cells including breast, cervical, endometrial and ovarian cancer (Makrydimas et al., 2003). This property seems to be correlated with the metastatic potential of these cells. Depending on the CD44 isoform and the cell background, various phenomena are possible. Therefore, HA interactions and signaling may differ among cancer types.

Adhesion is by no means a passive task. Rather, ligand binding, as exemplified for CD44 and other similar adhesion molecules, initiates a cascade of events that can be started by adherence to the extracellular matrix. This leads to activation of the molecule itself, binding to additional ligands, such as growth factors and matrix degrading enzymes, complex formation with additional transmembrane molecules and association with cytoskeletal elements and signal transducing molecules. Thus, through the interplay of CD44 with its ligands and associating molecules CD44 modulates adhesiveness, motility, matrix degradation, proliferation and cell survival, features that together may well allow a tumor cell to proceed through all steps of the metastatic cascade (Marhaba et al., 2004).

The interaction of CD44 with fragmented hyaluronan on rheumatoid synovial cells induces expression of VCAM-1 and Fas on the cells, which leads to Fas-mediated apoptosis of synovial cells by the interaction of T cells bearing FasL. On the other hand, engagement of CD44 on tumor cells derived from lung cancer reduces Fas expression and Fas-mediated apoptosis, resulting in less susceptibility of the cells to CTL-mediated cytotoxicity through Fas-FasL pathway (Yasuda et al., 2002). Therefore, the response to HA or its fragments cannot always be predicted. Patients may differ in their responses.

Versican is a large chondroitin sulfate proteoglycan produced by several tumor cell types, including malignant melanoma. The expression of increased amounts of versican in the extracellular matrix may play a role in tumor cell growth, adhesion and migration. V3 acts by altering the hyaluronan-CD44 interaction (Serra et al., 2005). In addition, multiple myeloma (MM) plasma cells express the receptor for hyaluronan-mediated motility (RHAMM), a hyaluronan-binding, cytoskeleton and centrosome protein. Expression and splicing of RHAMM are important molecular determinants of the disease severity of MM (Maxwell et al., 2004).

However, prior to the present invention, there was not a reliable supply of individual nanoHA sizes for investigating their effects on particular types of cancer.

Rapid blood vessel growth into the newly formed bone tissue is of paramount importance (Mowlem, 1963; Boume, 1972). Absence of adequate nutrient nourishment of the cells residing at the interior of large scaffolds after been implanted to a bone defect site will result in the death of the implanted cells and consequently the severe decrease of the possibility of bone regeneration. Apart from providing nutrients, rapid vascularization of bone grafts assists in the recruitment of osteoprogenitor and osteoclastic cells from the host tissue that will initiate the bone regeneration and remodeling cascade. The degradation products of hyaluronic acid (HA), oligoHA, are also known to stimulate endothelial-cell proliferation and to promote neovascularization associated with angiogenesis (West et al., 1985; Slevin et al., 2002).

Partial degradation products of sodium hyaluronate produced by the action of testicular hyaluronidase induced an angiogenic response (formation of new blood vessels) on the chick chorioallantoic membrane. Neither macromolecular hyaluronate nor exhaustively digested material had any angiogenic potential. Fractionation of the digestion products established that the activity was restricted to hyaluronate fragments between 4 and 25 disaccharides in length (West et al., 1985).

A delayed revascularization model was used previously to assess the angiogenic activity of hyaluronan fragments on impaired wound healing (Lees et al., 1995). 1- to 4-kDa hyaluronan fragments increased blood flow and increased graft vessel growth, whereas 33-kDa fragments had no such effect on graft blood flow or vessel growth.

In addition, Slevin et al. (2002) disclosed that angiogenic oligosacharides of hyaluronan induced multiple signaling pathways affecting vascular endothelial cell mitogenic and wound healing responses. Treatment of bovine aortic endothelial cells with oligosaccharides of hyaluronan (o-HA) resulted in rapid tyrosine phosphorylation and plasma membrane translocation of phospholipase Cγ1 (PLCγ1). Cytoplasmic loading with inhibitory antibodies to PLCγ1, Gβ, and Gα(i/o/t/z) inhibited activation of extracellular-regulated kinase 1/2 (ERK1/2). Treatment with the Gα(i/o) inhibitor, pertussis toxin, reduced o-HA-induced PLCγ1 tyrosine phosphorylation, protein kinase C (PKC) α and β1/2 membrane translocation, ERK1/2 activation, mitogenesis, and wound recovery, suggesting a mechanism for o-HA-induced angiogenesis through G-proteins, PLCγ1, and PKC. The work of Slevin et al. (2002) demonstrated a possible role for PKCα in mitogenesis and PKCβ1/2 in wound recovery, and that o-HA-induced bovine aortic endothelial cell proliferation, wound recovery, and ERK1/2 activation were also partially dependent on Ras activation.

Different cells in different tissues have different signalling pathways (due to varied levels and/or components that make each cell type distinct); thus, the effect of HA and oligosaccharides cannot be predicted. Empirical testing for each tissue is thus indicated. In addition, prior to the present invention, there was not a reliable supply of individual nanoHA sizes for investigating their effects.

Chemoenzymatic synthesis, however, employing catalytic glycosyltransferases with exquisite control and superb efficiency is currently being developed by several universities and companies. A major obstacle is the production of useful catalyst because the vast majority of glycosyltransferases are rare membrane proteins that are not particularly robust. In the copending applications referenced herein and in the presently claimed and disclosed invention, several practical catalysts from Pasteurella bacteria that allow for the synthesis of the three most important human GAGs (i.e., the three known acidic GAGs) are described and enabled (e.g., HA, chondroitin, and heparin).

All of the known HA, chondroitin and heparan sulfate/heparin glycosyltransferase enzymes that synthesize the alternating sugar repeat backbones in microbes and in vertebrates utilize UDP-sugar precursors and divalent metal cofactors (e.g., magnesium, cobalt, and/or manganese ion) near neutral pH according to the overall reaction:
nUDP-GlcUA+nUDP-HexNAc→2nUDP+[GlcUA-HexNAc]ₙ
where HexNAc=GlcNAc or GalNAc. Depending on the specific GAG and the particular organism or tissue examined, the degree of polymerization, n, ranges from about 25 to about 10,000. If the GAG is polymerized by a single polypeptide, the enzyme is called a synthase or co-polymerase.

As outlined in copending and incorporated by reference in the “Cross-Reference” section of this application hereinabove, the present applicant(s) have discovered four new dual-action enzyme catalysts from distinct isolates of the Gram-negative bacterium Pasteurella multocida using various molecular biology strategies. P. multocida infects fowl, swine, and cattle as well as many wildlife species. The enzymes are: a HA synthase, or pmHAS; a chondroitin synthase, or pmCS; and two heparosan synthases, or pmHS1 and pmHS2. To date, no keratan synthase from any source has been identified or reported in the literature.

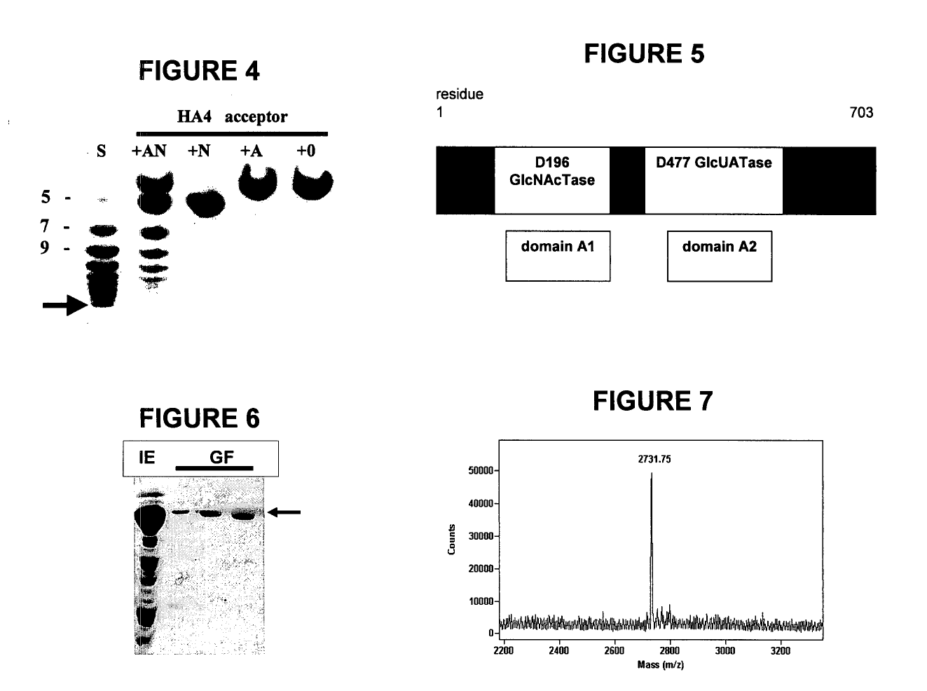

In copending U.S. Ser. No. 10/217,613, filed Aug. 12, 2002, the contents of which are hereby expressly incorporated herein by reference in their entirety, the molecular directionality of pmHAS synthesis was disclosed and claimed. pmHAS is unique in comparison to all other existing HA synthases of Streptococcus bacteria, humans and an algal virus. Specifically, recombinant pmHAS can readily elongate exogeneously-supplied short HA chains (e.g., 2-4 sugars) into longer HA chains (e.g., 3 to 150 sugars). The pmHAS synthase has been shown to add monosaccharides one at a time in a step-wise fashion to the growing chain (FIG. 4). The pmHAS enzyme's exquisite sugar transfer specificity results in the repeating sugar backbone of the GAG chain. The pmCS enzyme, which is about 90% identical at the amino acid level to pmHAS, performs the same synthesis reactions but transfers GalNAc instead of GlcNAc. The pmCS enzyme was described and enabled in copending U.S. Ser. No. 11/042,530, the contents of which are hereby expressly incorporated herein by reference in their entirety. The pmHS1 and pmHS2 enzymes are not very similar at the amino acid level to pmHAS, but perform similar synthesis reactions; the composition of sugars is identical but the linkages differ because heparosan is β4GlcUA-α4GlcNAc. The pmHS1 and PmHS2 enzymes were described and enabled in copending U.S. Ser. No. 10/142,143.

The explanation for the step-wise addition of sugars to the GAG chain during biosynthesis was determined by analyzing mutants of the pmHAS enzyme. pmHAS possesses two independent catalytic sites in one polypeptide (FIG. 5). Mutants were created that transferred only GlcUA, and distinct mutants were also created that transferred only GlcNAc. These mutants cannot polymerize HA chains individually, but if the two types of mutants are mixed together in the same reaction with an acceptor molecule, then polymerization was rescued. The chondroitin synthase, pmCS, has a similar sequence and similar two-domain structure. The heparosan synthases, pmHS1 and PmHS2, also contain regions for the two active sites. Single action mutants have also been created for the chondroitin synthase, pmCS, and are described hereinafter in detail.

The naturally occuring Pasteurella GAG synthases are very specific glycosyltransferases with respect to the sugar transfer reaction; only the correct monosaccharide from the authentic UDP-sugar is added onto acceptors. The epimers or other closely structurally related precursor molecules (e.g., UDP-glucose) are not utilized. The GAG synthases do, however, utilize certain heterologous acceptor sugars. For example, pmHAS will elongate short chondroitin acceptors with long HA chains. pmHS1 will also add long heparosan chains onto HA acceptor oligosaccharides as well as heparin oligosaccharides (see parent application U.S. Ser. No. 10/642,248). Therefore, the presently claimed and disclosed invention encompasses a wide range of hybrid or chimeric GAG oligosaccharides prepared utilizing these P. multocida GAG catalysts.

As used herein, the term “nucleic acid segment” and “DNA segment” are used interchangeably and refer to a DNA molecule which has been isolated free of total genomic DNA of a particular species. Therefore, a “purified” DNA or nucleic acid segment as used herein, refers to a DNA segment which contains a Hyaluronate Synthase (“HAS”) coding sequence or Chondroitin Synthase (“CS”) coding sequence or Heparin/Heparosan Synthase (“HS”) coding sequence yet is isolated away from, or purified free from, unrelated genomic DNA, for example, total Pasteurella multocida. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified pmHAS or pmCS or pmHS1 or PmHS2 gene refers to a DNA segment including HAS or CS or HS coding sequences isolated substantially away from other naturally occurring genes or protein encoding sequences. In this respect, the term “gene” is used for simplicity to refer to a functional protein-, polypeptide- or peptide-encoding unit. As will be understood by those in the art, this functional term includes genomic sequences, cDNA sequences or combinations thereof. “Isolated substantially away from other coding sequences” means that the gene of interest, in this case pmHAS or pmCS or pmHS1 or PmHS2 forms the significant part of the coding region of the DNA segment, and that the DNA segment does not contain other non-relevant large portions of naturally-occurring coding DNA, such as large chromosomal fragments or other functional genes or DNA coding regions. Of course, this refers to the DNA segment as originally isolated, and does not exclude genes or coding regions later added to, or intentionally left in, the segment by the hand of man.

Due to certain advantages associated with the use of prokaryotic sources, one will likely realize the most advantages upon isolation of the HAS or CS or HS gene from the prokaryote P. multocida. One such advantage is that, typically, eukaryotic genes may require significant post-transcriptional modifications that can only be achieved in a eukaryotic host. This will tend to limit the applicability of any eukaryotic HAS or CS or HS gene that is obtained. Moreover, those of ordinary skill in the art will likely realize additional advantages in terms of time and ease of genetic manipulation where a prokaryotic enzyme gene is sought to be employed. These additional advantages include (a) the ease of isolation of a prokaryotic gene because of the relatively small size of the genome and, therefore, the reduced amount of screening of the corresponding genomic library and (b) the ease of manipulation because the overall size of the coding region of a prokaryotic gene is significantly smaller due to the absence of introns. Furthermore, if the product of the pmHAS or pmCS or pmHS1 or PmHS2 gene (i.e., the enzyme) requires posttranslational modifications, these would best be achieved in a similar prokaryotic cellular environment (host) from which the gene was derived.

Preferably, DNA sequences in accordance with the present invention will further include genetic control regions which allow the expression of the sequence in a selected recombinant host. The genetic control region may be native to the cell from which the gene was isolated, or may be native to the recombinant host cell, or may be an exaggerous segment that is compatible with and recognized by the transcriptional machinery of the selected recbominant host cell. Of course, the nature of the control region employed will generally vary depending on the particular use (e.g., cloning host) envisioned.

Particular sequences that may be utilized in accordance with the presently claimed and disclosed invention were originally disclosed in detail in parent application U.S. Ser. No. 10/642,248. The individual sequences and their corresponding SEQ ID NO's are listed in Table II. The numbering, mutations and nomenclature used in Table II to describe each of the sequences is defined in detail in the parent application, which has previously been incorporated by reference.

In particular embodiments, the invention concerns utilizes DNA segments and recombinant vectors incorporating DNA sequences which encode a pmHAS or pmCS or pmHS1 or PmHS2 gene, that includes within its amino acid sequence an amino acid sequence in accordance with SEQ ID NO:2,4,6,8,10,12-22 or 25, respectively. Moreover, in other particular embodiments, the invention concerns isolated DNA segments and recombinant vectors incorporating DNA sequences which encode a gene that includes within its nucleic acid sequence an amino acid sequence encoding HAS or CS or HS pepetides or peptide fragment thereof, and in particular to a HAS or CS or HS peptide or peptide fragment thereof, corresponding to Pasteurella multocida HAS or CS or HS. For example, where the DNA segment or vector encodes a full length HAS or CS or HS protein, or is intended for use in expressing the HAS or CS or HS protein, preferred sequences are those which are essentially as set forth in SEQ ID NO:1, 3, 5, 7, 9, or 11, respectively.

Truncated pmHAS gene (such as, but not limited to, pmHAS¹⁻⁷⁰³, SEQ ID NO:11) also falls within the definition of preferred sequences as set forth above. For instance, at the carboxyl terminus, approximately 270-272 amino acids may be removed from the sequence and still have a functioning HAS. Those of ordinary skill in the art would appreciate that simple amino acid removal from either end of the pmHAS sequence can be accomplished. The truncated versions

TABLE II
DNA and Amino Acid Sequences Utilized in Accordance
with the Present Invention

| SEQ ID NO: | Sequence |
|---|---|
| 1 | pmHAS nucleic acid sequence |
| 2 | pmHAS amino acid sequence |
| 3 | pmCS nucleic acid sequence |
| 4 | pmCS amino acid sequence |
| 5 | pmHS1 nucleic acid sequence |
| 6 | pmHS1 amino acid sequence |
| 7 | bioclone of pmHS1 nucleic acid sequence |
| 8 | bioclone of pmHS1 amino acid sequence |
| 9 | pmHS2 nucleic acid sequence |
| 10 | pmHS2 amino acid sequence |
| 11 | pmHAS¹⁻⁷⁰³ nucleic acid sequence |
| 12 | pmHAS¹⁻⁷⁰³ amino acid sequence |
| 13 | pmHAS⁴⁶⁻⁷⁰³ |
| 14 | pmHAS⁷²⁻⁷⁰³ |
| 15 | pmHAS⁹⁶⁻⁷⁰³ |
| 16 | pmHAS¹¹⁸⁻⁷⁰³ |
| 17 | pmHAS¹⁻⁷⁰³ D247N |
| 18 | pmHAS¹⁻⁷⁰³ D249N |
| 19 | pmHAS¹⁻⁷⁰³ D527N |
| 20 | pmHAS¹⁻⁷⁰³ D529N |
| 21 | pmHAS¹⁻⁷⁰³ D247N D249N |
| 22 | pmHAS¹⁻⁷⁰³ D527N D529N |
| 23 | Motif I (GlcUA transferase portion) |
| 24 | Motif II (GlcNAc transferase portion) |
| 25 | pmCS¹⁻⁷⁰⁴ |

of the sequence (as disclosed hereinafter) simply have to be checked for HAS activity in order to determine if such a truncated sequence is still capable of producing HA. The other GAG synthases disclosed and claimed herein are also amenable to truncation or alteration with preservation of activity and such truncated or alternated GAG synthases also fall within the scope of the present invention.

Nucleic acid segments having HAS or CS or HS activity may be isolated by the methods described herein. The term “a sequence essentially as set forth in SEQ ID NO:X” means that the sequence substantially corresponds to a portion of SEQ ID NO:X and has relatively few amino acids or codons encoding amino acids which are not identical to, or a biologically functional equivalent of, the amino acids or codons encoding amino acids of SEQ ID NO:X. The term “biologically functional equivalent” is well understood in the art and is further defined in detail herein, as a gene having a sequence essentially as set forth in SEQ ID NO:X, and that is associated with the ability of prokaryotes to produce HA or a hyaluronic acid or chondroitin or heparin polymer in vitro or in vivo. In the above examples “X” refers to either SEQ ID NO: 1, 2, 3, 4, 5, 6, 7, 8, 9, 10, 11 or 12 or any additional sequences set forth herein, such as the truncated or mutated versions of pmHAS¹⁻⁷⁰³ that are contained generally in SEQ ID NOS:13-22.

The art is replete with examples of practitioner's ability to make structural changes to a nucleic acid segment (i.e., encoding conserved or semi-conserved amino acid substitutions) and still preserve its enzymatic or functional activity when expressed. See for special example of literature attesting to such: (1) Risler et al. “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (I) Ile and Val; (ii) Leu and Met, (iii) Lys, Arg, and Gln, and (iv) Tyr and Phe.”]; (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1:216-226 (1992) [“Analysis of the pattern of observed substitutions as a function of local environment shows that there are distinct patterns . . . ” Compatible changes can be made.]

These references and countless others indicate that one of ordinary skill in the art, given a nucleic acid sequence or an amino acid, could make substitutions and changes to the nucleic acid sequence without changing its functionality (specific examples of such changes are given hereinafter and are generally set forth in SEQ ID NOS:13-22). Also, a substituted nucleic acid segment may be highly identical and retain its enzymatic activity with regard to its unadulterated parent, and yet still fail to hybridize thereto. Additionally, the present application discloses 4 enzymes and numerous mutants of these enzymes that still retain at least 50% of the enzymatic activity of the unmutated parent enzyme—i.e., 1/2 of the dual action transferase activity of the unadulterated parent. As such, variations of the sequences and enzymes that fall within the above-defined functional limitations have been disclosed and enabled. One of ordinary skill in the art, given the present specification, would be able to identify, isolate, create, and test DNA sequences and/or enzymes that produce natural or chimeric or hybrid GAG molecules. As such, the presently claimed and disclosed invention should not be regarded as being solely limited to the specific sequences disclosed herein.

The present invention utilizes nucleic acid segments encoding an enzymatically active HAS or CS or HS from P. multocida—pmHAS, pmCS, pmHS1, and PmHS2, respectively. One of ordinary skill in the art would appreciate that substitutions can be made to the pmHAS or pmCS or pmHS1 or PmHS2 nucleic acid segments listed in SEQ ID NO: 1, 3, 5, 7, 9, and 11, respectively, without deviating outside the scope and claims of the present invention. Indeed, such changes have been made and are described in detail in the parent application U.S. Ser. No. 10/642,248 with respect to the mutants produced. Standardized and accepted functionally equivalent amino acid substitutions are presented in Table III. In addition, other analogous or homologous enzymes that are functionally equivalent to the disclosed synthase sequences would also be appreciated by those skilled in the art to be similarly useful in the methods of the present invention, that is, a new method to control precisely the size distribution of polysaccharides, namely glycosaminoglycans.

TABLE III

| | Conservative and Semi-Conservative |
|---|---|
| Amino Acid Group | Substitutions |
| NonPolar R Groups | Alanine, Valine, Leucine, Isoleucine, |
| | Proline, Methionine, Phenylalanine, |
| | Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

Another preferred embodiment of the present invention includes the use of a purified nucleic acid segment that encodes a protein in accordance with SEQ ID NO:1 or 3 or 5 or 7 or 9 or 11, respectively, further defined as a recombinant vector. As used herein, the term “recombinant vector” refers to a vector that has been modified to contain a nucleic acid segment that encodes an HAS or CS or HS protein, or fragment thereof. The recombinant vector may be further defined as an expression vector comprising a promoter operatively linked to said HAS- or CS- or HS-encoding nucleic acid segment.

A further preferred embodiment of the present invention includes the use of a host cell, made recombinant with a recombinant vector comprising an HAS or CS or HS gene. The preferred recombinant host cell may be a prokaryotic cell. In another embodiment, the recombinant host cell is an eukaryotic cell. As used herein, the term “engineered” or “recombinant” cell is intended to refer to a cell into which a recombinant gene, such as a gene encoding HAS or CS or HS, has been introduced mechanically or by the hand of man. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter associated or not naturally associated with the particular introduced gene.

In preferred embodiments, the HAS- or CS- or HS-encoding DNA segments further include DNA sequences, known in the art functionally as origins of replication or “replicons”, which allow replication of contiguous sequences by the particular host. Such origins allow the preparation of extrachromosomally localized and replicating chimeric or hybrid segments or plasmids, to which HAS- or CS- or HS-encoding DNA sequences are ligated. In more preferred instances, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The isolation and use of other replication origins such as the SV40, polyoma or bovine papilloma virus origins, which may be employed for cloning or expression in a number of higher organisms, are well known to those of ordinary skill in the art. In certain embodiments, the invention may thus be defined in terms of a recombinant transformation vector which includes the HAS- or CS- or HS-coding gene sequence together with an appropriate replication origin and under the control of selected control regions.

Thus, it will be appreciated by those of skill in the art that other means may be used to obtain the HAS or CS or HS gene or cDNA, in light of the present disclosure. For example, polymerase chain reaction or RT-PCR produced DNA fragments may be obtained which contain full complements of genes or cDNAs from a number of sources, including other strains of Pasteurella or from a prokaryot with similar glycosyltransferases or from eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HAS or CS or HS.

Once the DNA has been isolated, it is ligated together with a selected vector. Virtually any cloning vector can be employed to realize advantages in accordance with the invention. Typical useful vectors include plasmids and phages for use in prokaryotic organisms and even viral vectors for use in eukaryotic organisms. Examples include pKK223-3, pSA3, recombinant lambda, SV40, polyoma, adenovirus, bovine papilloma virus and retroviruses. However, it is believed that particular advantages will ultimately be realized where vectors capable of replication in both biotechnologically useful Gram-positive or Gram-negative bacteria (e.g., Bacillus, Lactococcus, or E. coli) are employed.

Vectors such as these, exemplified by the pSA3 vector of Dao and Ferretti or the pAT19 vector of Trieu-Cuot, et al., allow one to perform clonal colony selection in an easily manipulated host such as E. coli, followed by subsequent transfer back into a food grade Lactococcus or Bacillus strain for production of hyaluronan or chondroitin or heparin polymer. In another embodiment, the recombinant vector is employed to make the functional GAG synthase for in vitro use. These are benign and well studied organisms used in the production of certain foods and biotechnology products and are recognized as GRAS (generally recognized as safe) organisms. These are advantageous in that one can augment the Lactococcus or Bacillus strain's ability to synthesize HA or chondroitin or heparin through gene dosaging (i.e., providing extra copies of the HAS or CS or HS gene by amplification) and/or inclusion of additional genes to increase the availability of HA or chondroitin or heparin precursors. The inherent ability of a bacterium to synthesize HA or chondroitin or heparin can also be augmented through the formation of extra copies, or amplification, of the plasmid that carries the HAS or CS or HS gene. This amplification can account for up to a 10-fold increase in plasmid copy number and, therefore, the HAS or CS or HS gene copy number.

Another procedure to further augment HAS or CS or HS gene copy number is the insertion of multiple copies of the gene into the plasmid. Another technique would include integrating at least one copy of the HAS or CS or HS gene into chromosomal DNA. This extra amplification would be especially feasible, since the bacterial HAS or CS or HS gene size is small. In some scenarios, the chromosomal DNA-ligated vector is employed to transfect the host that is selected for clonal screening purposes such as E. coli, through the use of a vector that is capable of expressing the inserted DNA in the chosen host.

In certain other embodiments, the invention concerns the use of isolated DNA segments and recombinant vectors that include within their sequence a nucleic acid sequence essentially as set forth in SEQ ID NO:1, 3, 5, 7, 9, or 11. The term “essentially as set forth” in SEQ ID NO: 1, 3, 5, 7, 9, or 11 is used in the same sense as described above and means that the nucleic acid sequence substantially corresponds to a portion of SEQ ID NO: 1, 3, 5, 7, 9, or 11 and has relatively few codons which are not identical, or functionally equivalent, to the codons of SEQ ID NO: 1, 3, 5, 7, 9, or 11. The term “functionally equivalent codon” is used herein to refer to codons that encode the same amino acid, such as the six codons for arginine or serine, and also refers to codons that encode biologically equivalent amino acids, as set forth in Table III.

It will also be understood that amino acid and nucleic acid sequences may include additional residues, such as additional N- or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and enzyme activity is concerned. The addition of terminal sequences particularly applies to nucleic acid sequences which may, for example, include various non-coding sequences flanking either of the 5′ or 3′ portions of the coding region or may include various internal sequences, which are known to occur within genes. Furthermore, residues may be removed from the N- or C-terminal amino acids and yet still be essentially as set forth in one of the sequences disclosed herein, so long as the sequence meets the criteria set forth above, as well.

Allowing for the degeneracy of the genetic code as well as conserved and semi-conserved substitutions, sequences which have between about 40% and about 99%; or more preferably, between about 80% and about 90%; or even more preferably, between about 90% and about 99% identity to the nucleotides of SEQ ID NO: 1, 3, 5, 7, 9, or 11 will be sequences which are “essentially as set forth” in SEQ ID NO: 1, 3, 5, 7, 9, or 11. Sequences which are essentially the same as those set forth in SEQ ID NO: 1, 3, 5, 7, 9, or 11 may also be functionally defined as sequences which are capable of hybridizing to a nucleic acid segment containing the complement of SEQ ID NO: 1, 3, 5, 7, 9, or 11 under “standard stringent hybridization conditions”, “moderately stringent hybridization conditions,” “less stringent hybridization conditions,” or “low stringency hybridization conditions.” Suitable “standard” or “less stringent” hybridization conditions will be well known to those of skill in the art and are clearly set forth hereinbelow. In a preferred embodiment, standard stringent hybridization conditions or less stringent hybridization conditions are utilized.

The terms “standard stringent hybridization conditions,” “moderately stringent conditions,” and “less stringent hybridization conditions” or “low stringency hybridization conditions” are used herein, describe those conditions under which substantially complementary nucleic acid segments will form standard Watson-Crick base-pairing and thus “hybridize” to one another. A number of factors are known that determine the specificity of binding or hybridization, such as pH; temperature; salt concentration; the presence of agents, such as formamide and dimethyl sulfoxide; the length of the segments that are hybridizing; and the like. There are various protocols for standard hybridization experiments. Depending on the relative similarity of the target DNA and the probe or query DNA, then the hybridization is performed under stringent, moderate, or under low or less stringent conditions.

The hybridizing portion of the hybridizing nucleic acids is typically at least about 14 nucleotides in length, and preferably between about 14 and about 100 nucleotides in length. The hybridizing portion of the hybridizing nucleic acid is at least about 60%, e.g., at least about 80% or at least about 90%, identical to a portion or all of a nucleic acid sequence encoding a HAS or chondroitin or heparin synthase or its complement, such as SEQ ID NO: 1, 3, 5, 7, 9, or 11 or the complement thereof. Hybridization of the oligonucleotide probe to a nucleic acid sample typically is performed under standard or stringent hybridization conditions. Nucleic acid duplex or hybrid stability is expressed as the melting temperature or Tₘ, which is the temperature at which a probe nucleic acid sequence dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC, SSPE, or HPB). Then, assuming that 1% mismatching results in a 1 EC decrease in the Tₘ, the temperature of the final wash in the hybridization reaction is reduced accordingly (for example, if sequences having >95% identity with the probe are sought, the final wash temperature is decreased by about 5EC). In practice, the change in Tₘ can be between about 0.5EC and about 1.5EC per 1% mismatch. Examples of standard stringent hybridization conditions include hybridizing at about 68EC in 5×SSC/5× Denhardt's solution/1.0% SDS, followed with washing in 0.2×SSC/0.1% SDS at room temperature or hybridizing in 1.8×HPB at about 30EC to about 45EC followed by washing a 0.2-0.5×HPB at about 45EC. Moderately stringent conditions include hybridizing as described above in 5×SSC\5× Denhardt's solution 1% SDS washing in 3×SSC at 42EC. The parameters of salt concentration and temperature can be varied to achieve the optimal level of identity between the probe and the target nucleic acid. Additional guidance regarding such conditions is readily available in the art, for example, by Sambrook et al., 1989, Molecular Cloning, A Laboratory Manual, (Cold Spring Harbor Press, N.Y.); and Ausubel et al. (eds.), 1995, Current Protocols in Molecular Biology, (John Wiley & Sons, N.Y.). Several examples of low stringency protocols include: (A) hybridizing in 5×SSC, 5× Denhardts reagent, 30% formamide at about 30° C. for about 20 hours followed by washing twice in 2×SSC, 0.1% SDS at about 30° C. for about 15 min followed by 0.5×SSC, 0.1% SDS at about 30° C. for about 30 min (FEMS Microbiology Letters, 2000, vol. 193, p. 99-103); (B) hybridizing in 5×SSC at about 45° C. overnight followed by washing with 2×SSC, then by 0.7×SSC at about 55° C. (J. Viological Methods, 1990, vol. 30, p. 141-150); or (C) hybridizing in 1.8×HPB at about 30° C. to about 45° C.; followed by washing in 1×HPB at 23° C.

Naturally, the present invention also encompasses the use of DNA segments which are complementary, or essentially complementary, to the sequences set forth in SEQ ID NO:1 or 3 or 5 or 7 or 9 or 11. Nucleic acid sequences which are “complementary” are those which are capable of base-pairing according to the standard Watson-Crick complementarity rules. For example, the sequence 5′-ATAGCG-3′ is complementary to the sequence 5′-CGCTAT-3″ because when the two sequences are aligned, each “T” is able to base-pair with an “A”, which each “G” is able to base pair with a “C”. As used herein, the term “complementary sequences” means nucleic acid sequences which are substantially complementary, as may be assessed by the nucleotide comparison set forth above, or as defined as being capable of hybridizing to the nucleic acid segment of SEQ ID NO: 1, 3, 5, 7, or 9, or 11 under standard stringent, moderately stringent, or less stringent hybridizing conditions.

The nucleic acid segments utilized in the methods of the present invention, regardless of the length of the coding sequence itself, may be combined with other DNA sequences, such as promoters, polyadenylation signals, additional restriction enzyme sites, multiple cloning sites, epitope tags, polyhistidine regions, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

Naturally, it will also be understood that this invention is not limited to the use of the particular amino acid and nucleic acid sequences of any of SEQ ID NOS:1-25. Recombinant vectors and isolated DNA segments may therefore variously include the HAS or CS or HS coding regions themselves, coding regions bearing selected alterations or modifications in the basic coding region, or they may encode larger polypeptides which nevertheless include HAS or CS or HS coding regions or may encode biologically functional equivalent proteins or peptides which have variant amino acid sequences.

The DNA segments utilized in accordance with the present invention encompass DNA segments encoding biologically functional equivalent HAS or CS or HS proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the enzyme activity or to antigenicity of the HAS or CS or HS protein or to test HAS or CS or HS mutants in order to examine HAS or CS or HS activity at the molecular level or to produce HAS or CS or HS mutants having changed or novel enzymatic activity and/or sugar substrate specificity.

Traditionally, chemical or physical treatments of polysaccharides were required to join two dissimilar materials. For example, a reactive nucleophile group of one polymer or surface was exposed to an activated acceptor group of the other material. Two main problems exist with this approach, however. First, the control of the chemical reaction cannot be refined, and differences in temperature and level of activation often result in a distribution of several final products that vary from lot to lot preparation. For instance, several chains may be cross-linked in a few random, ill-defined areas, and the resulting sample is not homogenous. Second, the use of chemical reactions to join molecules often leaves an unnatural or nonbiological residue at the junction of biomaterials. For example, the use of an amine and an activated carboxyl group would result in an amide linkage. This inappropriate residue buried in a carbohydrate may pose problems with biological systems such as the subsequent production of degradation products which accumulate to toxic levels or the triggering of an immune response.

The methods for enzymatically producing defined glycosaminoglycan polymers utilized in the present invention involves providing at least one functional acceptor and at least one recombinant glycosaminoglycan transferase capable of elongating the functional acceptor in a controlled or repetitive fashion to form extended glycosaminoglycan-like molecules. At least one of UDP-GlcUA, UDP-GalUA UDP-GlcNAc, UDP-Glc, UDP-GalNAc, UDP-GlcN, UDP-GalN and a structural variant or derivative thereof is added in a stoichiometric ratio to the functional acceptor to provide glycosaminoglycan polymers that are substantially monodisperse in size.

The term “substantially monodisperse in size” as used herein will be understood to refer to defined glycoasminoglycan polymers that have a very narrow size distribution. For example, substantially monodisperse glycosaminoglycan polymers having a molecular weight in a range of from about 3.5 kDa to about 0.5 MDa will have a polydispersity value (i.e., Mw/Mn, where Mw is the average molecular weight and Mn is the number average molecular weight) in a range of from about 1.0 to about 1.1, and preferably in a range from about 1.0 to about 1.05. In yet another example, substantially monodisperse glycosaminoglycan polymers having a molecular weight in a range of from about 0.5 MDa to about 4.5 MDa will have a polydispersity value in a range of from about 1.0 to about 1.5, and preferably in a range from about 1.0 to about 1.2. For small sugar chains, oligosaccharides, the molecule can be exactly described structurally; these single molecular entities have a precise molecular weight, composition, and sugar linkages, and are thus considered “defined”.

Therefore, the term “defined” as used herein will be understood to refer to a single molecular entity having a precise molecular weight, composition and sugar linkages, and which is substantially free of other molecular entities having different molecular weights, compositions and sugar linkages.

The synthesis methods of the present invention allow natural and artificial oligosaccharides to be synthesized in a pure and defined state. In particular, immobilized mutatnt enzymes are very useful for step-wise synthesis. For example, the schemes of the presently disclosed and claimed invention can produce, for example but not by way of limitation, the defined oligosaccharides HA13, HA14 or HA20 with molecular weights of 2494 Da, 2670 Da, or 3808 Da, respectively (see FIG. 26). Such pure chemoenzymatically synthesized oligosaccharides are defined herein as “nanoHA”.

The functional acceptor utilized in accordance with the present invention will have at least two sugar units of uronic acid and/or hexosamine, wherein the uronic acid may be GlcUA, IdoUA or GalUA, and the hexosamine may be GlcNAc, GalNAc, GlcN or GalN. In one embodiment, the functional acceptor may be an HA oligosaccharide of about 3 sugar units to about 4.2 kDa, or an HA polymer having a mass of about 3.5 kDa to about 2MDa. In another embodiment, the functional acceptor may be a chondroitin oligosaccharide or polymer, a chondroitin sulfate oligosaccharide or polymer, or a heparosan-like polymer. In yet another embodiment, the functional acceptor may be an extended acceptor such as HA chains, chondroitin chains, heparosan chains, mixed glycosaminoglycan chains, analog containing chains or any combination thereof.

Any recombinant glycosaminoglycan transferase described or incorporated by reference herein may be utilized in the methods of the present invention. For example, the recombinant glycosaminoglycan transferase utilized in accordance with the present invention may be a recombinant hyaluronan synthase, a recombinant chondroitin synthase, a recombinant heparosan synthase, or any active fragment or mutant thereof. The recombinant glycosaminglycan transferase may be capable of adding only one UDP-sugar described herein above or may be capable of adding two or more UDP-sugars described herein above.

Metastasis, the escape of cancer cells throughout the body, is one of the biggest fears of both the ailing patient and the physician, and this area is a well studied application with respect to HA involvement. The present invention is directed to the use of defined, specific GAG molecules as a supplemental treatment to inhibit cancer growth and metatasis in conjunction with existing cancer therapies.

HA oligosaccharide treatment of cancer cell lines in culture reduced their rate of proliferation (Zeng et al., 1998). HA oligosaccharides were also very promising in an in vivo assay for tumor growth and metastasis (Zeng et al., 1998). In this assay, mice were injected with an invasive and virulent tumor cell line, and the progression of disease (e.g., general health, number of tumors, size of tumors) was monitored at a 10 day timepoint. Treatment with HA oligosaccharides greatly reduced the number and the size of tumors. Untreated animals would need to be euthanized within 2-4 weeks because of tremendous tumor growth. Various cancer cell lines, including melanoma, glioma, carcinomas from lung, breast and ovary, are susceptible to the therapeutic action of HA oligosaccharides.

The putative mode of action of the HA-oligosaccharide sugars is thought to be mediated by binding or interacting with one of several important HA-binding proteins (probably CD44 or RHAMM) in the mammalian body (Zeng et al., 1998; Yu et al., 1997; Bartolazzi et al., 1994; Zawadzki et al., 1998; Lesley et al., 2000; Radotra et al., 1997; Ahrens et al., 2001; Harada et al., 2001; Zhang et al., 1995; and Tan et al., 2001). However, the molecular details are lacking at this time, but there are several hypotheses. One attractive scenario for the anticancer action of HA-oligosaccharide is that multiple CD44 protein molecules in a cancer cell can bind simultaneously to a long HA polymer (Zeng et al., 1998; Yu et al., 1997; Bartolazzi et al., 1994; and Tan et al., 2001). This multivalent HA binding causes CD44 activation (perhaps mediated by dimerization or a receptor patching event) that triggers cancer cell activation and migration (FIG. 1). However, if the cancer cell is flooded with small HA-oligosaccharides, then each CD44 molecule individually binds a different HA molecule in a monovalent manner so that no dimerization/patching event occurs. Thus no activation or migration signal is transmitted to the cell.

It has been also shown that treatment with certain anti-CD44 antibodies (Yu et al., 1997; Bartolazzi et al., 1994; and Zawadzki et al., 1998) or CD44-antisense nucleic acid (Harada et al., 2001) prevents the growth and metastasis of cancer cells in a fashion similar to HA-oligosaccharides; in comparison to the sugars, however, these protein-based and nucleic acid-based reagents are somewhat difficult to deliver in the body and/or may have long-term negative effects. The optimal HA-sugar size was thought to be 10 to 14 sugars; molecules less than 8 sugars long do not have detectable biological activity (Zeng et al., 1998; and Tammi et al., 1998). A very desirable attribute of HA-oligosaccharides for therapeutics is that these sugar molecules are natural by-products that occur in small amounts in the healthy human body during the degradation of HA polymer; no untoward innate toxicity, antigenicity, or allergenic concerns are obvious (Zeng et al., 1998). The major current problem facing the development of the HA-based sugar therapeutics is that only very small amounts can be prepared by the current technology of the prior art.

The size of the hyaluronan (HA) polysaccharide dictates its biological effect in many cellular and tissue systems based on many reports in the literature. However, no source of very defined, uniform HA polymers with sizes greater than 5 kDa is currently available. This situation is complicated by the observation that long and short HA polymers appear to have antagonistic or inverse effects on some biological systems. Therefore, HA preparations containing a mixture of both size populations may yield contradictory or paradoxical results. One embodiment of the novel method of the present invention produces HA with very narrow, monodisperse size distributions that are referred to herein as “selectHA.”

The Pasteurella bacterial HA synthase enzyme, pmHAS, catalyzes the synthesis of HA polymers utilizing monosaccharides from UDP-sugar precursors in vivo and in vitro. pmHAS will also elongate exogenously supplied HA oligosaccharide acceptors in vitro; in fact, HA oligosaccharides substantially boost the overall incorporation rate. A purified, recombinant pmHAS derivative was employed herein to produce either native composition HA or derivatized HA.

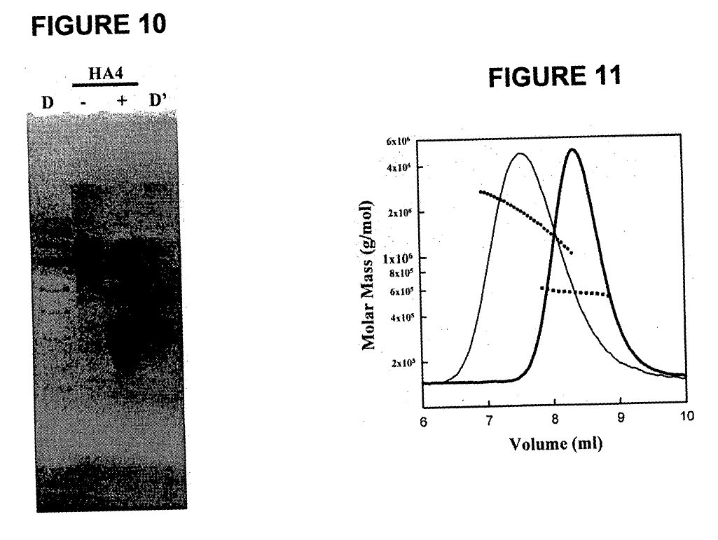

HA polymers of a desired size were constructed by controlling stoichiometry (i.e., ratio of precursors and acceptor molecules). The polymerization process is synchronized in the presence of acceptor, thus all polymer products are very similar (see FIGS. 10-17). In contrast, without the use of an acceptor, the polymer products are polydisperse in size. In the present examples, stoichiometrically controlled synchronized synthesis reactions yielded a variety of HA preparations in the range of ˜15 kDa to about 1.5 MDa. Each specific size class had a polydispersity value in the range of 1.01 for polymers up to 0.5 MDa or ˜1.2 for polymers of ˜1.5 MDa (1 is the ideal monodisperse size distribution) as assessed by size exclusion chromatography/multi-angle laser light scattering analysis. The selectHA preparations migrate on electrophoretic gels (agarose or polyacrylamide) as very tight bands.

The use of a modified acceptor allows the synthesis of selectHA polymers containing radioactive (e.g., ³H, ¹²⁵I), fluorescent (e.g., fluorescein, rhodamine), detection (i.e., NMR or X-ray), affinity (e.g., biotin) or medicant tags (see FIG. 16). In this scheme, each molecule has a single detection agent located at the reducing terminus. Alternatively, the use of radioactive UDP-sugar precursors allows the synthesis of uniformly labeled selectHA polymers with very high specific activities.

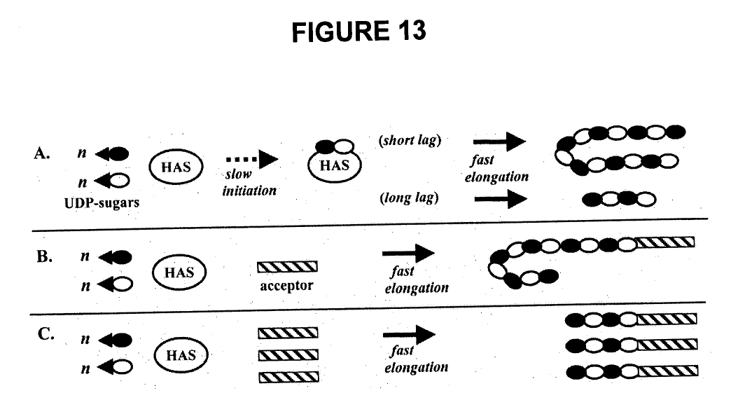

Overall, the selectHA reagents should assist in the elucidation of the numerous roles of HA in health and disease due to their monodisperse size distributions and defined compositions. It must be emphasized that unpredicted kinetic properties of the Pasteurella GAG synthases in a recombinant virgin state in the presence of defined, unnatural reaction conditions facilitates targeted size range production of monodisperse polymers that are not synthesizable by previously reported methods (FIG. 13).

The methods of the presently disclosed and claimed invention are novel and powerful, as the availability of gram quantities of these well-defined oligosaccharides is an important step in the development of small sugars as a new class of drugs for treatment of cancer metastasis. In addition to the anticancer effects, HA-based molecules promise to be useful for other areas as well, including but not limited to, stimulation of blood vessel growth (Rahmanian et al., 1997; and Lees et al., 1995) and stimulation of the immune system (Termeer et al., 2000; and Termeer et al., 2002).

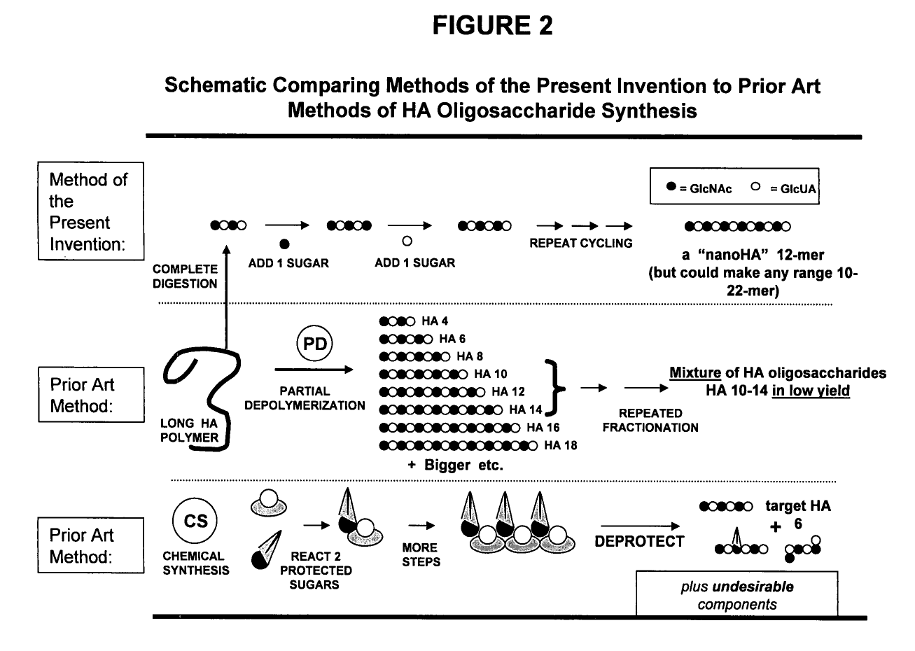

The most promising initial target oligosaccharides for inhibition of cancer metastasis are HA chains composed of 10 to 14 sugars. The two current prior art techniques for creating the desired HA-oligosaccharides are extremely limited and will not allow the medical potential of the sugars to be achieved (see FIG. 2 and Table IV). Small HA molecules are presently made either by: (1) partially depolymerizing (labeled PD in Table IV) costly large polymers with degradative enzymes (Zeng et al., 1998) or by chemical means (e.g., heat, acid, sonication), or (2) highly demanding organic chemistry-based carbohydrate synthesis (labeled CS) (Halkes et al., 1998). The former

TABLE IV
Comparison of the Methods of the Present Invention to Current Existing Technologies

| | | Current | | Innovative |
|---|---|---|---|---|
| | Present | Practice | Associated Barriers of | Approaches of the |
| Key Variable | Invention | (Prior Art) | Current Practice | Present Invention |
| Oligosaccharide | Require | Partial | Low yield for this size | Bioreactor system. |
| ultimate length | HA10-25 | depolymerization | range but obtainable | Sugar lengths from HA5 |
| | size for | [PD] | (need to harvest a | to HA150. For specific |
| | promising | | portion of Gaussian | target size of HA10-14, |
| | effects on | | peak). | relatively facile synthesis |
| | cancer. | Chemical | No report of sugars | on laboratory scale. |
| | | synthesis | bigger than HA6; | |
| | | [CS] | laborious and time- | |
| | | | consuming. | |
| Oligosaccharide | 90-100% | PD | Likely to contain | For each synthesis, |
| purity | pure, all | | contaminants of HA +/− two | only one major target |
| | correct | | sugar units unless | size molecule in final |
| | isomers, no | | do laborious repetitive | product; all natural |
| | undesired | | fractionation (causes low | sugars without |
| | foreign | | yields | undesirable |
| | moieties. | CS | Target molecule often | substituents or side |
| | | | has residual blocking | products. |
| | | | groups and some | |
| | | | racemization from | |
| | | | synthesis that may be | |
| | | | problematic. | |
| Synthesis speed | Minutes to | PD | Hours to days. | Enzyme synthesis rates |
| | hours time- | CS | Weeks to months. | 1-100 sugars per |
| | scale. | | | second; column format |
| | | | | allows high efficiency. |
| Flexibility of | Control at | PD | No flexibility; only HA | Sugar-by-sugar |
| final sugar | each | | sugars possible (unless | synthesis makes any HA |
| composition | synthetic | | chemically treated). | or chondroitin mixed |
| and structure | step to make | Reverse | Block hybrids possible; | structure; parallel |
| | novel | catalysis | hard to control particular | synthesis possible; |
| | structures | [RC] | desired structures. | designer |
| | (substitute | CS | Flexible, but each | oligosaccharides made |
| | with some | | synthesis requires | with no problem! |
| | non-HA | | unique strategy and | |
| | sugars) | | starting materials. | |

method is difficult to control, inefficient, costly, and is in a relatively stagnant development stage. For example, the enzyme wants to degrade the polymer to the 4 sugar end stage product, but this sugar is inactive. The use of acid hydrolysis also removes a fraction of the acetyl groups from the GlcNAc groups, thereby introducing a positive charge into an otherwise anionic molecule. The latter method, chemical synthesis, involves steps with low to moderate repetitive yield and has never been reported for a HA-oligosacchride longer than 6 sugars in length (Halkes et al., 1998). Also, racemization (e.g., production of the wrong isomer) during chemical synthesis may create inactive or harmful molecules. The inclusion of the wrong isomer in a therapeutic preparation in the past can have tragic consequences as evidenced by the birth defects spawned by the drug, Thalidomide. As sugars contain many similar reactive hydroxyl groups, in order to effect proper coupling between two sugars in a chemical synthesis, most hydroxyl groups must be blocked or protected. At the conclusion of the reaction, all of the protecting groups must be removed, but this process is not perfect; as a result, a fraction of the product molecules retain these unnatural moieties. The issues of racemization and side-products from chemical synthesis are not problems for the high-fidelity enzyme catalysts of the present invention.

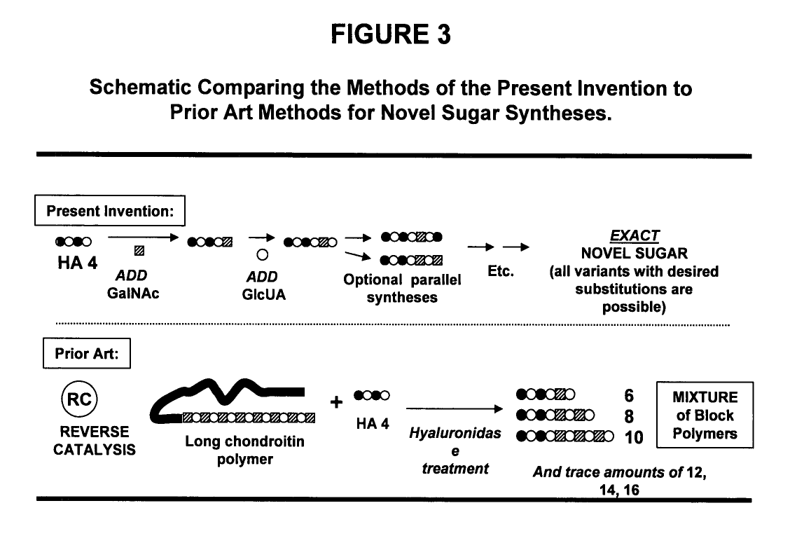

The partial depolymerization method only yields fragments of the original HA polymer and is essentially useless for creating novel sugars beyond simple derivatizations (e.g., esterifying some fraction of GlcUA residues in an indiscriminate fashion). Chemical synthesis (FIG. 2) could suffice in theory to make novel sugars, but the strategy needs to be customized for adding a new sugar, plus the problems with side-reactions/isomerization and the ultimate oligosaccharide size still pose the same trouble as described earlier. Another distinct prior art method using the degradative enzymes to generate small molecules by “running in reverse” (labeled RC in FIG. 3 and Table IV) on mixtures of two polymers (e.g., HA and chondroitin) has some potential for novel synthesis (Takagaki et al., 2000). However, this technology can make only a very limited scope of products with a block pattern (no single or specifically spaced substitutions possible) using slow reactions that cannot easily be customized or controlled. No other technology is as versatile as the biocatalytic system of the present invention with respect to flexibility of final oligosaccharide structure in the 8 to 14 sugar size range—this is truly an added value of the system of the presently disclosed and claimed invention. Novel, “designer” molecules can be prepared with minimal re-tooling by use of the appropriate enzyme catalysts and substrates described herein.

As described herein earlier, the present inventor has discovered the four Pasteurella glycosaminoglycan synthases. A novel strategy was used to isolate the gene for a HA synthase, pmHAS, as described in U.S. Ser. No. 10/217,613, filed Aug. 12, 2002, and this unique enzyme does not closely resemble the known HA synthases of Streptococcus bacteria, man or an algal virus. The chondroitin synthase, pmCS, was the first known enzyme to polymerize chondroitin (see U.S. Ser. No. 09/842,484, filed Apr. 25, 2002). The present inventor has demonstrated the molecular directionality of pmHAS synthesis, and it was observed that acceptor sugars were elongated by pmHAS if supplied with the appropriate UDP-sugar (FIG. 4). The acceptor sugar was elongated if supplied in a free state in a liquid solution or covalently immobilized to plastic (data not shown). These findings form the basis for oligosaccharide synthesis both in liquid phase (for bioreactor synthesis) and in solid phase (for microarray construction). The pmCS enzyme, which is about 90% identical at the amino acid level to pmHAS, performs the same synthesis reactions but incorporates GalNAc instead of GlcNAc. On the other hand, the Streptococcus, vertebrate, and virus HASs do not perform this reaction and are relatively useless for oligosaccharide synthesis.

The pmHAS polypeptide contains duplicated sequence elements that were considered to be sugar-transfer sites; one site would transfer a GlcNAc sugar and the other site would transfer a GlcUA sugar to form the alternating HA polymer backbone (FIG. 5). If a certain aspartate residue (e.g., D136) in the first domain, A1, was mutated, then the enzyme only transfers GlcUA. On the other hand, if a certain residue (e.g., D477) in the second domain, A2, was mutated, then the enzyme only transfers GlcNAc. Other essential amino acids may also be mutated in a similar fashion to achieve the same goal. The mutation of two groups in the same motif/domain are better for inactivating the dual action catalyst and transforming to a desirable single-action catalyst for immobilized reactors. Thus the pmHAS enzyme was molecularly dissected into its two catalytic components (see parent application U.S. Ser. No. 10/642,248). Based on the protein sequence, the chondroitin synthase, pmCS, also has 2 domains.

Further mutagenesis transformed the low expression level (˜0.1% of protein) pmHAS membrane protein found in nature to a high expression level (˜10% of protein) soluble protein (see parent application U.S. Ser. No. 10/642,248). This alteration of pmHAS allows both (i) the generation of more catalyst and (ii) the purification of catalyst by standard chromatographic means. Several strategies were developed to purify milligram-level quantities of pmHAS mutant proteins by conventional protein chromatography. 90-100% pure enzyme is obtained in one or two steps by the methods of the present invention (FIG. 6). All phases of purification are readily scaled up. A soluble version of the chondroitin synthase, pmCS, has also been produced (see parent application U.S. Ser. No. 10/642,248.

It has been shown that the pmHAS¹⁻⁷⁰³ enzyme responds very favorably with a linear increase in reaction rate when tested with high UDP-sugar concentrations (10-15 mM) predicted to be useful for “industrial” scale synthesis; the presence of two similar UDP-sugars simultaneously does not cause cross-inhibition (see DeAngelis et al., 2003). A property of many enzymes is that their reaction products or downstream metabolites often regulate the catalysis rate. In the live cell, this control makes sense because if sufficient product is made, then it is not logical to consume more starting materials. In biotechnology, however, this feedback inhibition prematurely shuts the enzyme system down, thereby reducing yields. HA synthases from both Streptococcus bacteria and man are turned off or inhibited by low levels of the unavoidable by-product of HA synthesis, UDP (0-5% activity at 0.1-0.4 mM). On the other hand, pmHAS¹⁻⁷⁰³ is not very susceptible to UDP inhibition (Table V). This fortunate circumstance allows higher production yields because UDP does not need to be vigorously removed during the reaction.

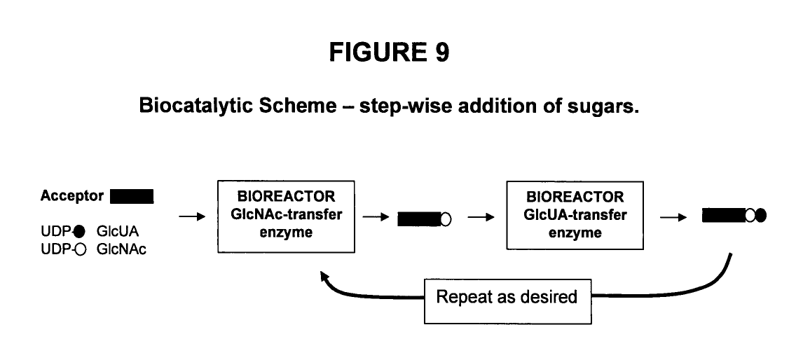

Large-scale synthesis mediated by catalysts can be performed in a variety of formats. Perhaps the most useful and advantageous method is the catalytic bioreactor format (FIG. 9). For example, processing often involves passing the starting material through a reactor column packed

TABLE V
Insensitivity of pmHAS¹⁻⁷⁰³ to UDP By-product Inhibition.
Radioactive [³H]HA₄ acceptor was incubated with pmHAS in a reaction
containing 1 mM UDP-GlcUA and 1 mM UDP-GlcNAc in the presence of
increasing amounts of free UDP. The amount of radioactivity incorporated
into high molecular weight product was measured. The sugar elongation
reaction proceeds very well even in the presence of high ratios of
UDP/UDP-sugar.

| | Polymer |
|---|---|
| UDP Level (mM) | Production (dpm) |
| 0 | 4,800 |
| 5 | 4,900 |
| 10 | 3,700 |
| 15 | 3,300 |

with catalyst. This column serves to hold or to immobilize the catalyst (often an extremely expensive material) so that it can contact all of the starting material in a serial fashion. After the reaction occurs in the column bed, the product exits the column. A good column (i.e., one that does not lose the catalyst or allow the catalyst to fail) allows repetitive (multiple use allows cost-savings) or continuous reactions to occur.

In designing the biocatalytic system for sugar synthesis of the present invention, it was first tested if the pmHAS enzyme and its mutant derivatives could be immobilized to a bead suitable for use in a column. Chemistry that will allow virtually 100% of the purified enzyme to be attached to a bead with minimal loss of catalytic activity (data not shown) was identified. The beads with wild-type dual-action pmHAS made long HA polymer chains. The mutant versions of pmHAS possessing only a single functional transfer site transferred only one type of sugar (see FIG. 9). Furthermore, the immobilized enzyme was extremely stable and retained catalytic function even if maintained at useful functional temperatures (i.e., 30° C.) for a week in reaction buffer.

Laboratory-Scale Pilot Synthesis with Bioreactors. Two bioreactors with immobilized mutant pmHAS enzymes were prepared (described above). One column only transferred GlcNAc while the other column transferred only GlcUA. As an easily monitorable test, a series of fluorescent HA oligosaccharides were prepared with these bioreactors. As a feedstock, a fluorescent HA4 (F-HA4) acceptor was first made in a two-step chemical synthesis. This acceptor and the two required UDP-sugars, UDP-GlcNAc and UDP-GlcUA (0.8 mM each), together in a suitable reaction buffer (1 M ethylene glycol, 10 mM MnCl₂, 50 mM Tris, pH 7.2) were applied to the two enzyme columns in a repetitive fashion 8 times (4 cycles each column). Samples of the reaction mixture were analyzed by thin layer chromatography at every step. It was observed that larger oligosaccharides were made as expected. A desirable nanoHA molecule, a F-HA12 sugar, was produced in a single afternoon. The identity of the product was verified by the most rigorous analytical method, mass spectrometry (FIG. 7) (Zaia et al., 2001). The theoretical molecular weight for the F-HA12 sugar agreed with the observed experimental molecular weight (2731.8 Da).

In addition to being a sensitive test molecule for the synthesis process of the present invention, this fluorescent reagent has an added bonus for use as a probe. The fluorescent tag allows sensitive visualization of the location and the fate (e.g., stick to cell surface, internalized, etc.) of nanoHA on live cancer cells. The reagent also demonstrates that a drug can be coupled to HA oligosaccharides by the methods of the present invention.

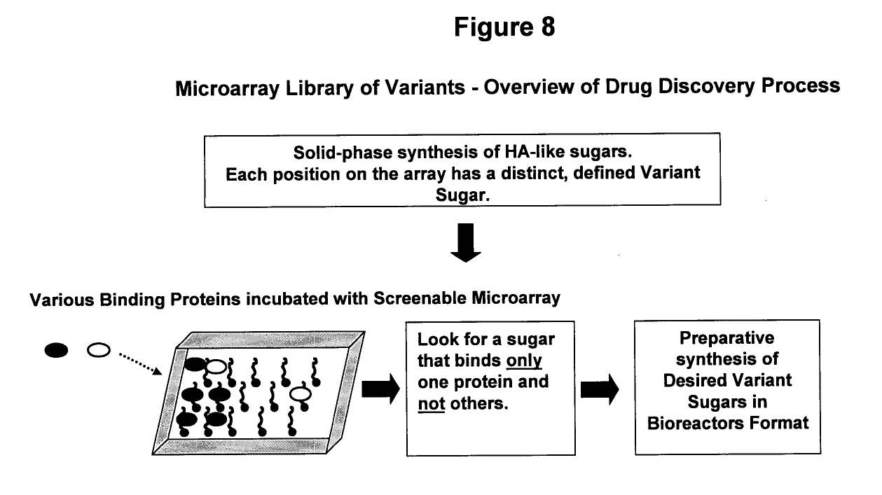

Microarrays are emerging as powerful, high-throughput tools in genomics and proteomics research. Sugar-based microarrays can be generated by the methods of the present invention to test a wide variety of novel oligosaccharides for interaction with proteins essential for tissue integrity or recognition/signaling events. Information from screening microarrays allows for production of GAGs with increased potency and/or increased selectivity that can also be synthesized in the bioreactor. As shown in FIG. 8, HA polymers may be synthesized in situ to a glass slide compatible for analysis with conventional microarray detection instrumentation. For oligosaccharide production, the individual sugars would be added in a controlled, stepwise fashion to build custom oligosaccharides.

Acceptor-mediated Synchronization of Reaction Yields Monodisperse HA Products—Recombinant pmHAS synthesizes HA chains in vitro if supplied with both required UDP-sugars (DeAngelis et al., 1998) according to the equation:
nUDP-GlcUA+nUDP-GlcNAc→2nUDP+[GlcUA-GlcNAc]ₙ
However, if a HA-like oligosaccharide ([GlcUA-GlcNAc]x) is also supplied in vitro, then the overall incorporation rate was elevated up to ˜50- to 100-fold (DeAngelis, 1999). It was suggested that the rate of initiation of a new HA chain de novo was slower than the subsequent elongation (i.e., repetitive addition of sugars to a nascent HA molecule). The observed stimulation of synthesis by exogenous acceptor appears to operate by bypassing the kinetically slower initiation step allowing the elongation reaction to predominate as in the following equation:
nUDP-GlcUA+nUDP-GlcNAc+[GlcUA-GlcNAc]x→2nUDP+[GlcUA-GlcNAc]x+n
HA polymerization reactions were performed with purified pmHAS and UDP-sugar precursors under various conditions and analyzed the reaction products by agarose gel electrophoresis and/or size exclusion chromatography with MALLS. It was observed that the size distribution of HA products obtained was quite different depending on the presence or the absence of the HA4 acceptor; in summary, reactions with acceptor produced smaller HA chains with a more narrow size distribution. An example is depicted in FIGS. 10 and 11 where the reaction containing HA4 acceptor yielded a HA product with a Mw (weight average molecular mass) of 555 kDa and polydispersity (Mw/Mn; Mn=number average molecular weight) of 1.006, but the parallel reaction without acceptor resulted in product with a Mw of 1.8 MDa and Mw/Mn of 1.17. For reference, the polydispersity value for an ideal monodisperse polymer equals 1.

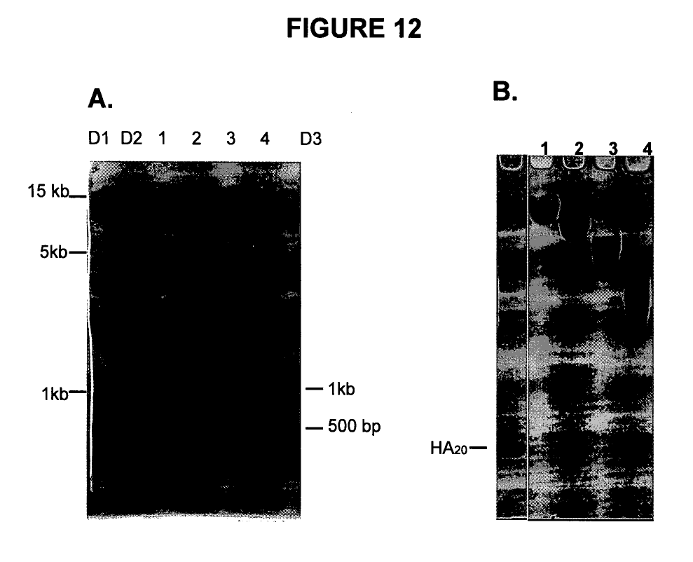

To verify whether pmHAS can utilize HA acceptors of various sizes, parallel assays were set up using the same starting conditions, and at various times additional UDP-sugars were added to the reaction. The result indicated that intermediate products were utilized as starting material for later chain elongation by pmHAS. (FIG. 12).

To explain the findings above, it was hypothesized that polymerization by pmHAS in the presence of an HA acceptor is a synchronized process. Reactions without acceptor exhibit a lag period interspersed with numerous, out of step initiation events that yield a short HA oligosaccharide (FIG. 13A). Once any HA chain is formed, the polymer is elongated rapidly. Other new HA chains that arise later during the lag period are also elongated rapidly, but the size of these younger chains never catches up to the older chains in a reaction with a finite amount of UDP-sugars. In contrast, in reactions containing an acceptor, all HA chains are elongated in parallel in a nonprocessive fashion resulting in a more homogenous final polymer population (FIG. 13B). For practical synthesis where there are more acceptor molecules than catalyst molecules, it is critical that processive elongation (i.e., no dissociation of the nascent HA chain and the synthase until polymerization is complete) does not occur because disparity would arise when some acceptor chains are elongated before other chains.

Stoichiometric Control of HA Product Size—The two enzymological properties of recombinant pmHAS described above also allow for the control of HA polymer size in chemoenzymatic syntheses. First, as noted above, the rate-limiting step in vitro appears to be chain initiation. Therefore, pmHAS will transfer monosaccharides onto the existing HA acceptor chains before substantial de novo synthesis. Second, the enzyme polymerizes HA in a rapid nonprocessive fashion in vitro (Jing et al., 2000; and DeAngelis et al., 2003). Therefore, the amount of HA4 should affect the final size of the HA product when a finite amount of UDP-sugar is present. The synthase will add all available UDP-sugar precursors to the nonreducing termini of acceptors as in the equation:
nUDP-GlcUA+nUDP-GlcNAc+z[GlcUA-GlcNAc]ₓ→2nUDP+z[GlcUA-GlcNAc]_{x+(n/z)}
If there are many termini (i.e., z is large), then a limited amount of UDP-sugars will be distributed among many molecules and thus result in many short polymer chain extensions (FIG. 13C). Conversely, if there are few termini (i.e., z is small), then the limited amount of UDP-sugars will be distributed among few molecules and thus result in long polymer chain extensions (FIG. 13B).

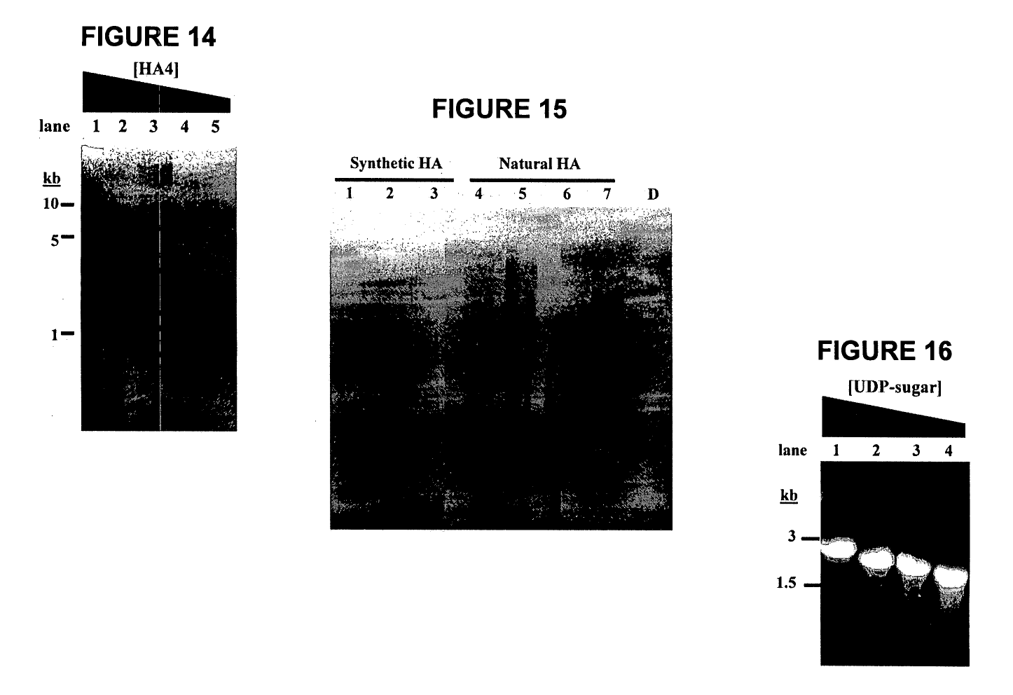

To test this speculation, a series of assays were performed utilizing various levels of HA4 with a fixed amount of UDP-sugar and pmHAS (FIG. 14). With this general strategy, HA was generated from 16 kDa to 2 MDa with polydispersity ranging from 1.001 to ˜1.2 (FIG. 15). By controlling the molar ratio of acceptor to UDP-sugar, it is now possible to select the final HA polymer size desired. Typically, ˜50% to ˜70% of the starting UDP-sugars are consumed in the reactions on the basis of HA polysaccharide recovery.

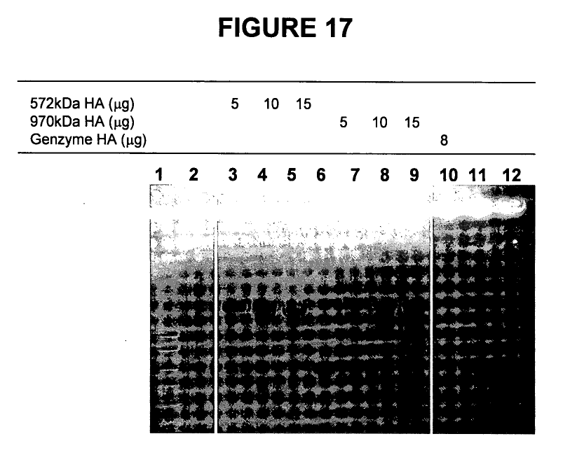

Interestingly, if an intermediate-sized molecular mass HA chain is prepared by this method, then the chain may be elongated by simply adding more UDP-sugars to the reaction mixture provided that active catalyst is present. The resulting polymers migrate as tight bands on gels and appear quite monodisperse throughout the entire reaction time course even after multiple additions of UDP-sugars. The resulting bands with steadily increasing molecular weights indicated that HA polymers larger than oligosaccharides (˜20 kDa to 1.3 MDa) may also be utilized as starting material for chain elongation by pmHAS (FIG. 17).

In vitro synthesis of tagged or labeled HA—The technology of the present invention for the production of monodisperse polymers also allows the use of a modified acceptor to synthesize HA polymers containing various types of foreign moieties. The pmHAS adds monosaccharides to the nonreducing terminus of the acceptor chain (DeAngelis, 1999), thus the aldehyde functionality of the reducing end is available for reaction by numerous chemical schemes. An example is shown using fluorescent HA4 acceptor to produce fluorescent monodisperse HA of various sizes (FIG. 16). Similarly, radioactive (e.g., ³H, ¹²⁵I), affinity (e.g., biotin), detection (e.g., probe for NMR or X-ray uses or a reporter enzyme), or medicant tagged glycosaminoglycan polymers are possible with the appropriate modified acceptor. However, the invention is not limited to the tags described herein, and other tags known to a person having ordinary skill in the art may be utilized in accordance with the present invention.

Alternatively, substitution of all or a portion of the unlabeled UDP-sugars in a chemoenzymatic synthesis reaction with a radioactive precursor (e.g., UDP-[³H]GlcUA) is a very useful method to produce labeled HA probes (data not shown). The advantage of this method is that the radioactive HA does not contain any foreign, non-sugar moieties that might interfere with biological function or cause mistargeting.

Utility of synthetic HA—The molecular weights of most commercially available HA preparations is usually in the 10⁵-10⁶ Da range (Laurent et al., 1992). For research requiring smaller HA polymers, degradation via enzymatic (e.g., hyaluronidase digestion) or chemical (e.g., radicals or oxidation) or physical (e.g., ultrasonication) methods are usually employed. However, this process is not always satisfactory because it is time-consuming, the final yield of the targeted HA size is low, and at least one demanding chromatographic step is usually required. The methods of the present invention can generate HA as small as ˜15 kDa with polydispersity (Mw/Mn) around 1.001 with the current synchronized stoichiometrically-controlled synthesis technique. If the synthesis of smaller monodisperse HA oligosaccharides (less than 25 monosaccharides long or ˜5 kDa) is required, then it is preferable to utilize a pair of reactors with immobilized mutant pmHAS enzymes (a GlcUA-transferase and a GlcNAc-transferase) operating in an alternating, repetitive fashion (DeAngelis et al., 2003).

High molecular weight HA preparations are commercially available from animal or bacterial sources, but inherent problems including possible contaminants and broad size distributions complicate research. Polydispersities of commercially available HA polymers are commonly higher than 1.5. Indeed, there exists a substantial need for uniform HA in biomedical studies (Uebelhart et al., 1999). The present invention has demonstrated that narrow size distribution, high molecular weight HA (˜1-2 MDa) is also readily prepared by synchronized, stoichiometrically-controlled reactions (FIG. 15). However, the present invention is not limited to such size HA, and other HA product size ranges are also within the scope of the present invention.

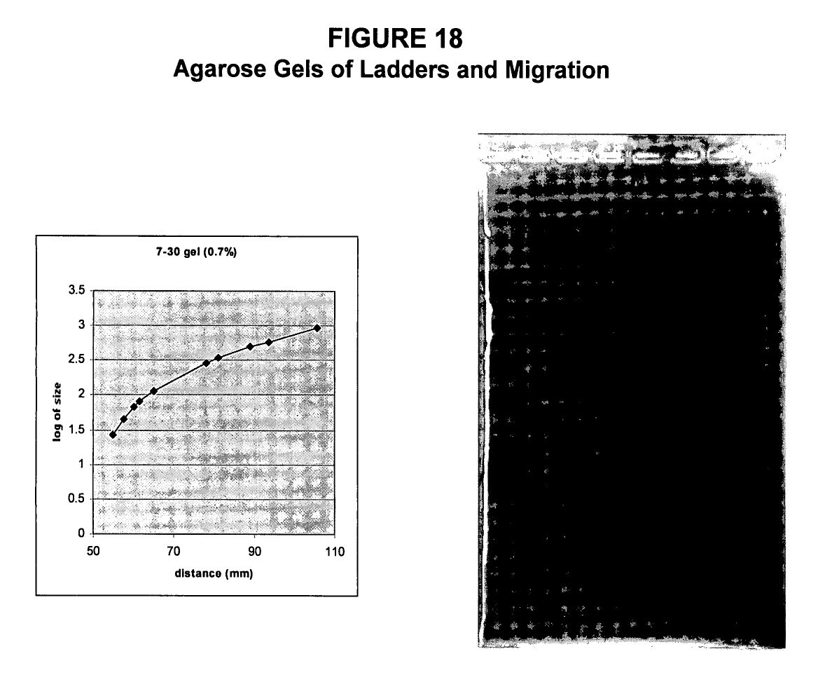

To determine the exact average molecular mass of large polymers of HA (>10 kDa), MALLS is usually the choice. Yet many researchers need to quickly estimate the molecular mass and lack the required instrumentation. The correlation of HA migration on agarose gels with DNA (Lee et al., 1994) is often used for this purpose. Drawbacks of this method include (i) the original “calibration standard” HA samples were not uniform or monodisperse, and (ii) the migration of HA and DNA on agarose gels changes differentially with alteration of the agarose concentration. Ladders comprised of an assortment of synthetic HA polymers with defined, narrow size distributions (FIGS. 15 and 18) provide an excellent series of standards for characterizing the size of HA in experimental samples.

In general, the unique technology platform of the presently disclosed and claimed invention allows the generation of a variety of improved synthetic HA tools with narrow size distributions and defined compositions for elucidating the numerous roles of HA in health and disease. Similar synchronized, stoichiometrically-controlled reactions utilizing the other Pasteurella glycosaminoglycan synthases (DeAngelis, 2002) is also within the scope of the presently disclosed and claimed invention, and allows the chemoenzymatic synthesis of monodisperse chondroitin and heparosan polymers.

In addition to the small sugar chains (e.g., tetrasaccharide HA4), larger HA polymers can be used as starting acceptor for pmHAS; the enzyme will elongate existing chains with more sugars. Experiments were performed using 575 kDa HA and 970 kDa HA (synthesized in vitro with pmHAS and HA4 as acceptor, using the previously described methods) and a commercially available HA sample (˜2 MDa; Genzyme) as acceptors. The results indicate that the existing HA chains were further elongated (FIG. 17). For example, the ˜2 MDa starting material in lane 11 was elongated to produce the larger (i.e., slower migrating) material in lane 10. Therefore, a method for creating higher value longer polymers is also described by the present invention. The length of the final product can be controlled stoichiometrically as shown in lanes 7-9; a lower starting acceptor concentration (lane 7) results in longer chains because the same limited amount of UDP-sugars is consumed, making a few long chains instead of many shorter chains (lane 9).

The molecular weights of naturally existing HA polymers usually range from hundreds of thousands up to several millions of Daltons. For research requiring smaller HA polymers, enzymatic degradation is usually the first choice. However, this process is not satisfactory because it is time-consuming and the final yield of the targeted HA size fraction is low, and demanding chromatography is required. With the in vitro synthesis techniques of the present invention, HA as small as 10 kDa can be generated with polydispersity around 1.001.

High molecular HAs are commercially available from animal or bacterial sources. Problems with those include possible contaminants leading to immunological responses as well as broad size distribution (Soltes etc, 2002). Polydispersities (Mw/Mn) are commonly higher than 1.5. Conclusions drawing from experimental data during biological research with these HA could be misleading. Thus there exists a need for uniform HA to perform biological study, as agreed by Uebelhart and Williams (1999).

In general, the unique technologies of the present invention allow the generation of a variety of defined, monodisperse HA tools for elucidating the numerous roles of HA in health and disease due to their monodisperse size distributions and defined compositions.

In addition to making HA polymers, the relaxed acceptor specificity of pmHAS allows the use of various chondroitin acceptors. This allows the production of monodisperse hybrid GAGs that have utility in medicine including tissue engineering and surgical aids. In particular, new protein-free proteoglycans are now possible that do not have antigenicity or allergenicity concerns compared to animal-derived products.

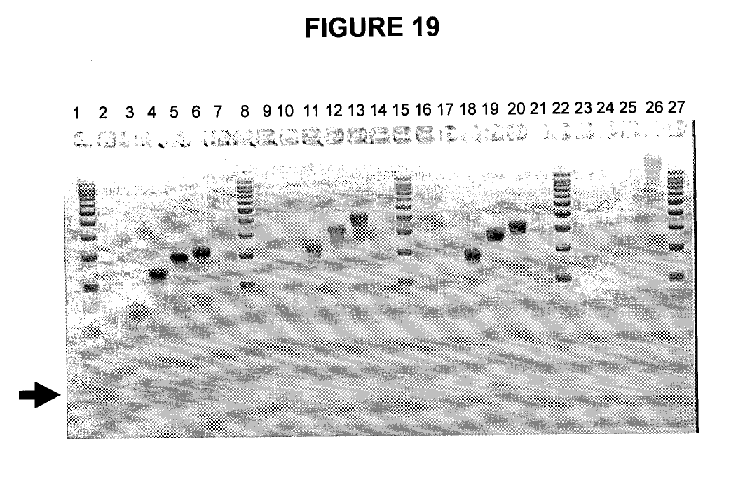

In FIG. 19, various monodisperse chondroitin sulfate HA hybrid GAGs are created by elongating a variety of chondroitin sulfates (A, B, and C) with pmHAS, thus adding HA chains. Various amounts of HA were added to the preparations (at various times during reaction as noted) by adding more UDP-sugars. For example, lanes 3-6 show hybrids with a constant amount of chondroitin sulfate and increasing HA chain lengths. The starting chondroitin sulfates stain weakly here, and the band position is marked with an arrow. Without the acceptor (lanes 23-26), no such defined bands are seen; after a long period, some HA polymer shows up (lane 26) which results from de novo initiation without acceptor.

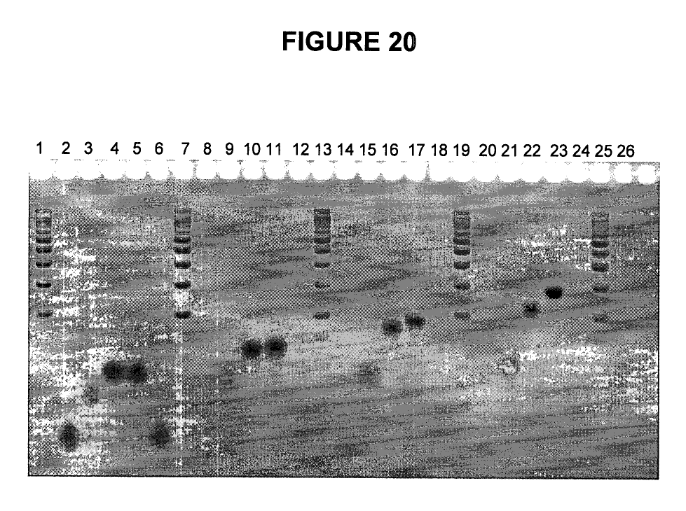

In FIG. 20, chondroitin sulfate A was elongated with pmHAS, thus adding HA chains. Various amounts of HA were added to the preparations by controlling the level of chondroitin acceptor (thus changing the UDP-sugar/acceptor ratio) as well as adding more UDP-sugars during the reaction. By changing the UDP-sugar/acceptor ratio, stoichiometric control of the hybrid GAG size was demonstrated.

In addition to extension with a HA synthase, other GAG synthases may be used in the methods of the present invention. For example, a chondroitin synthase such as but not limited to pmCS can be used to elongate an existing chondroitin sulfate polymer or HA polymer to produce defined hybrid GAG molecules of various structures. Again, these molecules may have use as surgical aids or tissue engineering scaffolds.

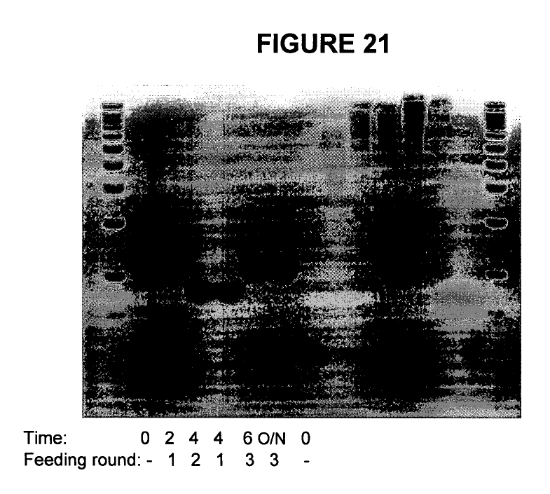

In FIG. 21, pmCS and UDP-GlcUA, UDP-GalNAc were reacted with either a 81 kDa HA acceptor (lanes 3-7) or no acceptor (lanes 9-13). Various lengths of chondroitin were added to the HA chains (at longer times with more UDP-sugars producing longer hybrid chains). Without the acceptor, no such defined bands were seen; after a long period, some long pure chondroitin polymer shows up which results from de novo initiation without acceptor.

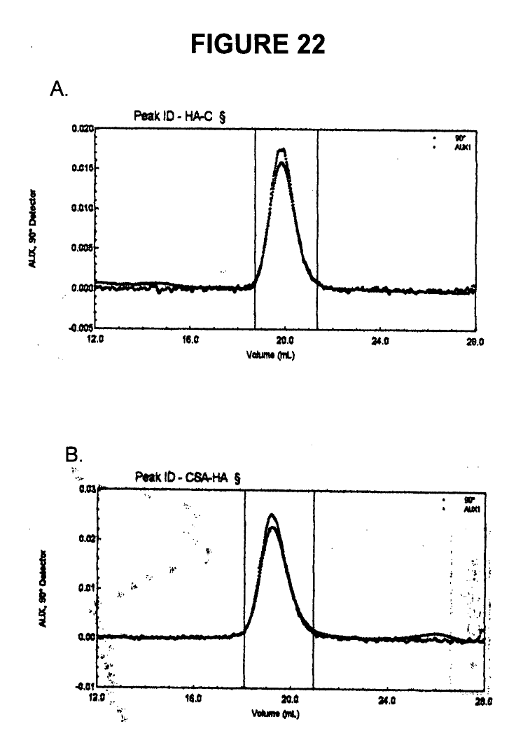

In FIG. 22, Size exclusion (or gel filtration) chromatography analysis coupled with multi-angle laser light scattering detection confirms the monodisperse nature of polymers created by the present invention. In the FIG. 22A, HA (starting MW 81 kDa) extended with chondroitin chains using pmCS (same sample used in FIG. 21, lane #7, overnight [O/N] extension) was analyzed; the material was 280,000 Mw and polydispersity (Mw/Mn) was 1.003+/−0.024. Chondroitin sulfate HA extended with HA chains using pmHAS (same sample used in FIG. 19, lane #23) was analyzed and shown in FIG. 22B; the material was 427,000 Mw and polydispersity (Mw/Mn) was 1.006+/−0.024.

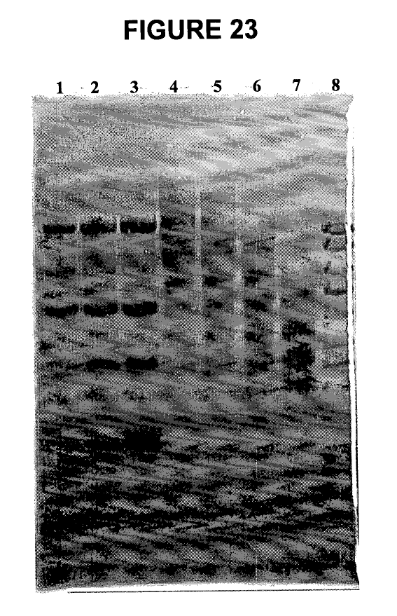

In FIG. 23, a 0.7% agarose gel detected with Stains-all compares the monodisperse, ‘select HA’ to commercially produced HA samples is shown. In lanes 1-3, the mixture of various monodisperse HAs made by the present invention (separate reaction products that were recombined to run all in one lane; sizes from top to bottom of lane: 1.27 MDa, 946 kDa, 575 kDa, 284 kDa, 27 kDa) run as discrete, tight bands. In contrast, in lanes 4-7, the commercially produced HA samples run as polydisperse smears (lane 4, 1.1 MDa; 5, 810 kDa; 6, 587 kDa; 7, 350 kDa). Remarkably, the monodisperse HA bands look almost as narrow as the single-molecule species of DNA present in lane 8 (BIOLINE standard).

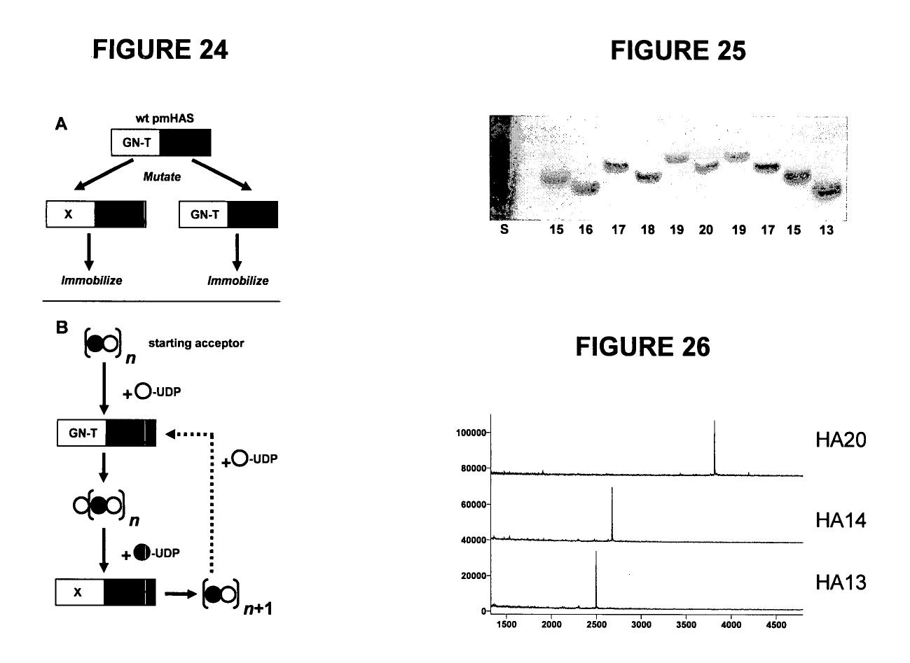

Generation of Immobilized Enzyme-Reactors —As mentioned previously, the good solubility and higher yields of pmHAS¹⁻⁷⁰³ compared to wild-type pmHAS allow for the purification of active HA synthase. Mutation of a predicted UDP-sugar substrate-binding amino acid motif, DXD (Jing et al., 2003), in either of the two enzyme active sites into NXN converts the dual-action HA synthase into essentially a single-action glycosyltransferase. Mutation of the A1 domain yields a β4GlcUA-Tase, while mutation of the A2 domain yields a β3GlcNAc-Tase (FIG. 24A). The pmHAS mutants that contained only a single change in a DXD motif (e.g., DXN or NXD) reported earlier were not suitable for preparative-scale synthesis because their HA polymerizing activity could be rescued partially by the high UDP-sugar concentration utilized (Jing et al., 2003). On the other hand, the NXN double mutants (SEQ ID NOS:21 and 22) were virtually inactive as HA synthases at the high substrate levels employed here.

Each of the pmHAS NXN mutant enzymes were purified and immobilized covalently onto activated agarose beads in a functional state. The solid-phase catalyst facilitates (a) recirculation of the reaction mixture to assure quantitative sugar addition at every step, (b) simplified recovery of the oligosaccharide product, and (c) preservation of the catalyst for subsequent steps. The enzyme immobilized on beads was also more stable than free soluble enzyme over time or heat challenge (data not shown).

Chemoenzymatic Synthesis—In the typical oligosaccharide synthesis, 1 equivalent of the tetrasaccharide HA4 (β4GlcUA-β3GlcNAc)₂ acceptor and 1.2 to 1.5 equivalents of UDP-sugar in reaction buffer were circulated over an enzyme reactor at room temperature (FIG. 24B). The reactions were virtually complete after one or two passes of the reaction mixture through a reactor (˜5 to 10 minutes) as judged by thin layer chromatography (TLC) (not shown). However, it is very important in any multistep or repetitive synthesis to assure virtual completion of each step to avoid accumulation of a multitude of failure products at the end of the process. Therefore, the reaction mixture was recirculated on a given enzyme reactor for an additional 1 to 2 hours. The reaction mixture was then removed from the first enzyme reactor, the next required UDP-sugar was added, and the reaction mixture was recirculated on the next enzyme reactor. No significant runaway polymerization (i.e., multiple sugar additions on a single reactor) was noted with these NXN mutant enzyme-reactors even in the presence of both UDP-sugars. No intermediate purification measures were performed during the 8, 9 or 10 sugar addition steps to produce HA12, HA13 or HA14, respectively. The total synthesis time was about two days. Cycling the desalted tridecasaccharide HA13 through seven more enzyme reactor steps created a longer oligosaccharide, the 20-mer HA20 (FIG. 25).

The crude reaction mixtures were judged to contain >95-97% of the target product oligosaccharide by matrix-assisted laser desorption ionization time-of-flight mass spectrometry (MALDI-TOF MS) (not shown) and polyacrylamide gel electrophoresis (FIG. 25). Therefore, each enzyme reactor step is proceeding to >99.5% of completion to achieve the overall observed operating efficiency.

The only final purification required was gel filtration chromatography to remove low molecular weight salts, unincorporated precursor sugar, and UDP byproduct from the target oligosaccharide. For the larger HA molecules, simple dialysis or ultrafiltration for desalting would suffice. All of the oligosaccharides had the expected masses as measured by MALDI-TOF MS (FIG. 26). The final yields after 10 addition steps at the 90 μmole-scale were about 50% due to losses during sample monitoring and slight retention of sugars on the agarose-based reactors in each cycle.

The recombinant Pasteurella enzyme, designated a Class II HA synthase, has several unique intrinsic properties that allow chemoenzymatic synthesis of desirable short oligosaccharides. In contrast, all the known Class I HA synthases (streptococcal, viral, and vertebrate) are relatively unsuitable for this synthetic task. Only pmHAS will readily elongate in vitro exogenously supplied oligosaccharides (e.g., HA4). The Class I HAS are not as well understood as pmHAS and the two component sugar transferase activities have not been separated in a practical fashion by molecular genetic means.

In the dual enzyme reactor strategy of the present invention, the final size of the oligosaccharide depends on the number of sugar addition steps employed. Substantial benefits of this scheme are that purification of intermediates is not needed after every step and that high stepwise yields are possible by recirculating the reaction mixture over a given enzyme-reactor. An added benefit of utilizing pmHAS derivatives for multistep syntheses is that these enzymes are relatively insensitive to the UDP byproduct of the transferase reaction (˜60% inhibition at 15 mM UDP with 1 mM substrates; Table V). In contrast, the class I HAS enzymes are greatly inhibited by relatively low concentrations of UDP (>90% inhibition at 0.5 mM UDP with 1 mM substrates). Indeed, the pmHAS mutants are efficient catalysts as judged by swift reaction times utilizing only 1.2 to 1.5 molar equivalents of UDP-sugar per sugar addition step.

Other methods for production of HA oligosaccharides have been reported, but they have shortcomings. Chemical synthesis of carbohydrates is difficult due to the demands of stereoselective (i.e., a versus b glycosidic linkages) and regioselective (i.e., only one of the multiple functionalities per sugar ring) coupling of sugars. State of the art synthetic strategies utilize multiple protection/deprotection cycles in a variety of toxic and/or flammable solvents with often less than quantitative yields (FIG. 2, “CS”). In contrast, the enzyme is the “perfect” carbohydrate chemist performing sugar additions with no side-products in aqueous solution. The largest HA oligosaccharide synthesized by chemical means to date was the hexasaccharide (HA6) containing a methoxyphenyl group at the reducing terminus (Halkes et al., 1998); a very nice example, but this product is too small for the interesting biological activities described earlier. Another major difficulty of organic synthesis is that the reaction rate for longer oligosaccharide formation is significantly slower than for shorter sugars. In contrast, the pmHAS-catalyzed reaction rate appears to increase for the longer HA oligosaccharide acceptors (not shown).

The cost of UDP-sugars used in chemoenzymatic synthesis, a once ominous barrier, has been significantly lowered recently. Recombinant permeabilized bacterial systems for the production of kilogram quantities of nucleotide-sugars are becoming available (Koizumi et al., 1998). Even though the costs of these fine biochemicals may be higher than simpler organic chemicals and synthetic reagents, the reduced number of reaction steps, the higher overall yields, and the avoidance of toxic materials lowers the overall economic differential between a ‘standard’ and a chemoenzymatic carbohydrate synthesis.

As noted earlier, the initial discovery experiments implicating that small HA chains had interesting biological properties utilized mixtures of oligosaccharides prepared by partial digestion of high molecular weight HA polysaccharide with degradative enzymes. Such protocols typically suffer from poor reproducibility and low yields of the target species (e.g., one length in range of HA10 to HA20). Some HA chains are cleaved too much (the limit digest is HA4) resulting in inactive fragments while other HA chains are not sufficiently fragmented resulting in longer molecules which will possibly counteract the desirable effect of the shorter target HA oligosaccharides. Recently, two groups have reported anion-exchange chromatography purification schemes to separate desirable HA oligosaccharides from partial digests (Tawada et al., 2002; and Mahoney et al., 2001). However, in these reports only HA-derived materials were isolated (i.e., no novel sugars), and the processes rely on chromatographic separations which may be difficult to scale up.

In addition to being an advance in carbohydrate synthesis, the presently disclosed and claimed invention also yields basic science knowledge with respect to elucidating the mechanism of GAG synthesis in Pasteurella. Two modes of polymer synthesis are possible: (a) processive (i.e., nascent polymer is retained by the glycosyltransferase until the chain is completed) or (b) non-processive (i.e., nascent polymer is repetitively bound and released by the glycosyltransferase). In our immobilized reactor format, the HA oligosaccharide must be bound transiently to a mutant synthase, extended by one sugar, and released before the oligosaccharide is acted on by a second mutant synthase. The rapidity and the efficiency of our chemoenzymatic synthesis implies that the pmHAS catalyst elongates the HA polymer in a non-processive fashion. To form the long HA polysaccharide chains (˜1×10³ sugars) observed in the Pasteurella bacterial capsule, other proteins or components of the polymer transport apparatus probably assist in vivo with chain retention because this property does not appear to be an intrinsic characteristic of pmHAS.

Previously, the present inventor has demonstrated that reactions containing a mixture of two mutant enzymes (i.e., a GlcNAc-Tase and a GlcUA-Tase) formed HA polymers relatively efficiently in comparison to wild-type (Jing et al., 2000; and Jing et al., 2003). One explanation for this observation is that two pmHAS monomers actually form the active catalytic species and the two polypeptides cooperate to perform the reaction; a lesion in any one site would be compensated by employing a pair of molecules. However, based on the success of the reactor synthesis, pmHAS must act as a monomer because the two mutant enzymes are immobilized in separate locations that cannot physically interact.

The chemoenzymatic route disclosed herein also allows the use of modified acceptor molecules. For example, previously the present inventor has elongated radiolabeled acceptor (HA4 reduced with borotritide) into longer HA chains (DeAngelis, 1999), but the foreign moiety at the reducing terminus of the HA polymer could instead be a drug or another polymer to enhance therapeutic effect. The pmHAS wild-type enzyme and pmHAS-based transferases described here only transfer authentic HA monosaccharides from UDP-sugars; the C4 epimer analogs (i.e., galactose-based) and UDP-glucose do not substitute (DeAngelis et al., 1998). Thus, the present invention also includes mutant enzymes suitable for reactors developed to catalyze the incorporation of unnatural sugars to form new molecules with altered biological activity and/or useful chemical properties Overall, the chemoenzymatic synthesis platform of the present invention opens up a wide spectrum of new biomedical applications, and is not limited simply to the creation of single molecular entities, such as HA12 through HA20.

It is well established that the large array of functions that a tumor cell has to fulfill to settle as a metastasis in a distant organ requires cooperative activities between the tumor and the surrounding tissue and that several classes of molecules are involved, such as cell-cell and cell-matrix adhesion molecules and matrix degrading enzymes, to name only a few. Furthermore, metastasis formation requires concerted activities between tumor cells and surrounding cells as well as matrix elements and possibly concerted activities between individual molecules of the tumour cell itself. CD44 transmembrane glycoproteins belong to the families of adhesion molecules and have originally been described to mediate lymphocyte homing to peripheral lymphoid tissues. It was soon recognized that the molecules, under selective conditions, may suffice to initiate metastatic spread of tumor cells (Marhaba et al., 2004). CD44 variant isoforms have been implicated in many biological processes, such as cell adhesion, cell substrate, cell to cell interactions, including lymphocyte homing haemopoiesis, cell migration and metastasis. These abilities are of great importance in chronic inflammation and in cancer. CD44 has shown the ability to recruit leucocytes to vascular endothelium at sites of inflammation, which is one of the first steps in the inflammatory response. In cancer, deregulation of the adhesion mechanisms increases the ability of tumor cells to metastasis. This behavior seems to be explained by the existing relationship between hyaluronan and CD44, which is its major cell surface receptor. There are CD44 variant isoforms (i.e., similar, but not functionally equivalent) which are expressed on different types of normal cells. In addition some isoforms are overexpressed on tumor cells including breast, cervical, endometrial and ovarian cancer (Makrydimas et al., 2003). This property seems to be correlated with the metastatic potential of these cells. Depending on the CD44 isoform and the cell background, various phenomena are possible. Therefore, HA interactions and signaling may differ among cancer types.

Adhesion is by no means a passive task. Rather, ligand binding, as exemplified for CD44 and other similar adhesion molecules, initiates a cascade of events that can be started by adherence to the extracellular matrix. This leads to activation of the molecule itself, binding to additional ligands, such as growth factors and matrix degrading enzymes, complex formation with additional transmembrane molecules and association with cytoskeletal elements and signal transducing molecules. Thus, through the interplay of CD44 with its ligands and associating molecules CD44 modulates adhesiveness, motility, matrix degradation, proliferation and cell survival, features that together may well allow a tumor cell to proceed through all steps of the metastatic cascade (Marhaba et al., 2004).

The interaction of CD44 with fragmented hyaluronan on rheumatoid synovial cells induces expression of VCAM-1 and Fas on the cells, which leads to Fas-mediated apoptosis of synovial cells by the interaction of T cells bearing FasL. On the other hand, engagement of CD44 on tumor cells derived from lung cancer reduces Fas expression and Fas-mediated apoptosis, resulting in less susceptibility of the cells to CTL-mediated cytotoxicity through Fas-FasL pathway (Yasuda et al., 2002). Therefore, the response to HA or its fragments cannot always be predicted. Patients may differ in their responses.

Versican is a large chondroitin sulfate proteoglycan produced by several tumor cell types, including malignant melanoma. The expression of increased amounts of versican in the extracellular matrix may play a role in tumor cell growth, adhesion and migration. V3 acts by altering the hyaluronan-CD44 interaction (Serra et al., 2005). In addition, multiple myeloma (MM) plasma cells express the receptor for hyaluronan-mediated motility (RHAMM), a hyaluronan-binding, cytoskeleton and centrosome protein. Expression and splicing of RHAMM are important molecular determinants of the disease severity of MM (Maxwell et al., 2004).

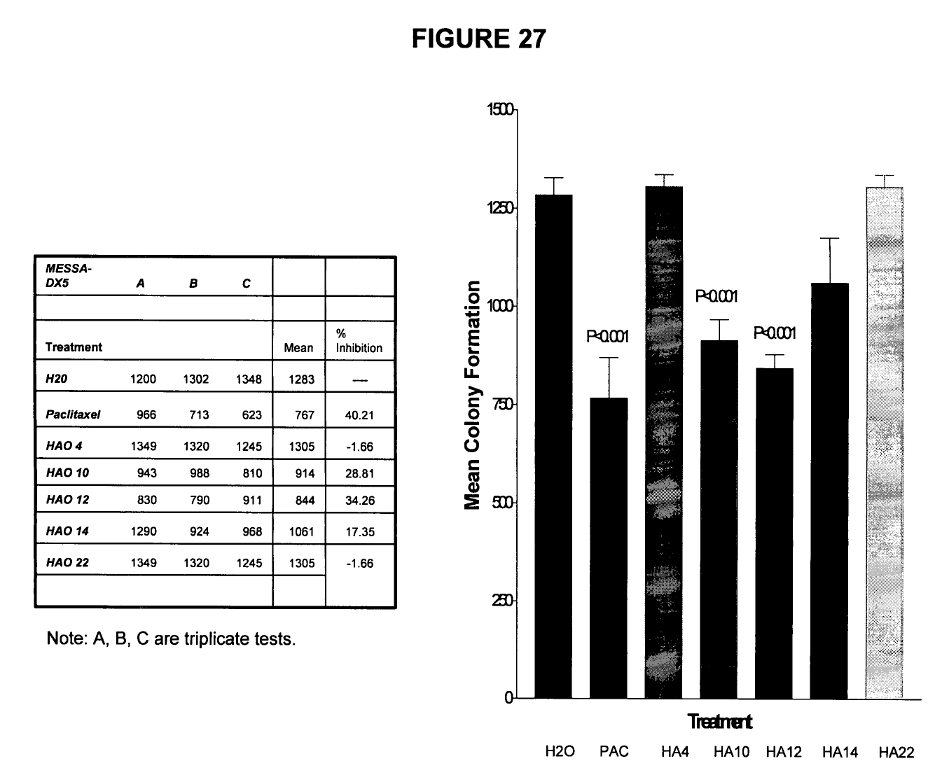

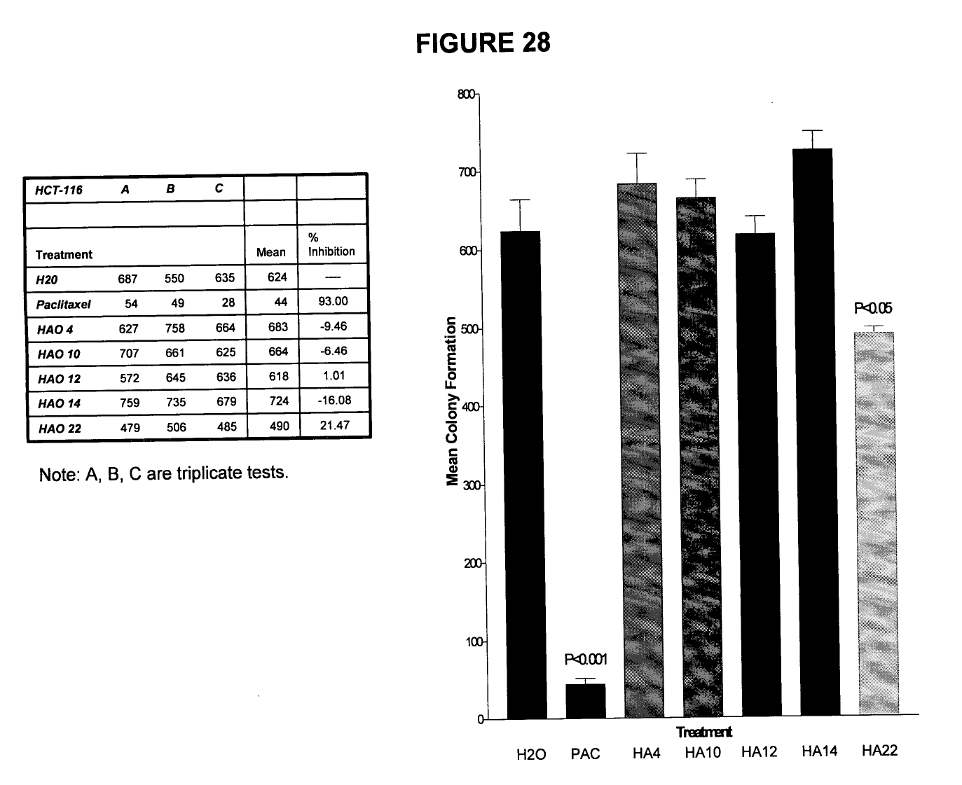

However, prior to the present invention, there was not a reliable supply of individual nanoHA sizes for investigating their effects on particular types of cancer. Therefore, the effects of different HA sizes on tumor cell growth was investigated. Anchorage independent growth, such as growth in soft agar, is a hallmark of transformation for those mammalian cells that usually require a substrate to which adhere in order to proliferate. Therefore, an inhibition of colony formation of a cancer cell line growing in soft agar is a direct measurement of the ability of a substance to inhibit cancer growth. Paclitaxel or nanoHA were used in standard soft agar growth test assays with two different cell lines: drug-resistance human uterine sarcoma MES-SA/Dx5 (FIG. 27) or human colon adenocarcinoma (FIG. 28). HA10 and HA12 caused inhibition of mean colony formation in MESSA-Dx5 cell line. However, no significant effect was seen with HA4, HA14, and HA22. In contrast, HA22 caused inhibition of mean colony formation in the HCT-116 cell line, while HA4, HA10, HA12 and HA14 had no effect. This demonstrates that two different tumor cell lines were inhibited by two different size HA products.

Rapid blood vessel growth into the newly formed bone tissue is of paramount importance (Mowlem, 1963; Boume, 1972). Absence of adequate nutrient nourishment of the cells residing at the interior of large scaffolds after been implanted to a bone defect site will result in the death of the implanted cells and consequently the severe decrease of the possibility of bone regeneration. Apart from providing nutrients, rapid vascularization of bone grafts assists in the recruitment of osteoprogenitor and osteoclastic cells from the host tissue that will initiate the bone regeneration and remodeling cascade. The degradation products of hyaluronic acid (HA), oligoHA, are also known to stimulate endothelial-cell proliferation and to promote neovascularization associated with angiogenesis (West et al., 1985; Slevin et al., 2002).

Partial degradation products of sodium hyaluronate produced by the action of testicular hyaluronidase induced an angiogenic response (formation of new blood vessels) on the chick chorioallantoic membrane. Neither macromolecular hyaluronate nor exhaustively digested material had any angiogenic potential. Fractionation of the digestion products established that the activity was restricted to hyaluronate fragments between 4 and 25 disaccharides in length (West et al., 1985).

A delayed revascularization model was used previously to assess the angiogenic activity of hyaluronan fragments on impaired wound healing (Lees et al., 1995). 1- to 4-kDa hyaluronan fragments increased blood flow and increased graft vessel growth, whereas 33-kDa fragments had no such effect on graft blood flow or vessel growth.

In addition, Slevin et al. (2002) disclosed that angiogenic oligosacharides of hyaluronan induced multiple signaling pathways affecting vascular endothelial cell mitogenic and wound healing responses. Treatment of bovine aortic endothelial cells with oligosaccharides of hyaluronan (o-HA) resulted in rapid tyrosine phosphorylation and plasma membrane translocation of phospholipase Cγ1 (PLCγ1). Cytoplasmic loading with inhibitory antibodies to PLCγ1, Gβ, and Gα(i/o/t/z) inhibited activation of extracellular-regulated kinase 1/2 (ERK1/2). Treatment with the Gα(i/o) inhibitor, pertussis toxin, reduced o-HA-induced PLCγ1 tyrosine phosphorylation, protein kinase C (PKC) α and β1/2 membrane translocation, ERK1/2 activation, mitogenesis, and wound recovery, suggesting a mechanism for o-HA-induced angiogenesis through G-proteins, PLCγ1, and PKC. The work of Slevin et al. (2002) demonstrated a possible role for PKCa in mitogenesis and PKCβ1/2 in wound recovery, and that o-HA-induced bovine aortic endothelial cell proliferation, wound recovery, and ERK1/2 activation were also partially dependent on Ras activation.

Different cells in different tissues have different signalling pathways (due to varied levels and/or components that make each cell type distinct); thus, the effect of HA and oligosaccharides cannot be predicted. Empirical testing for each tissue is thus indicated.

The chick embryo chorioallantoic membrane (CAM) is an extraembryonic membrane that is commonly used in vivo to study both new vessel formation and its inhibition in response to tissues, cells, or soluble factors (see Storgard et al., 2005). Quantitative or semiquantitative methods may be used to evaluate the amount of angiogenesis and anti-angiogenesis. Thanks to the CAM system, angiogenesis could be investigated in association with normal, inflammatory and tumor tissues, and soluble factors inducing angiogenic or anti-angiogenic effects could be identified.

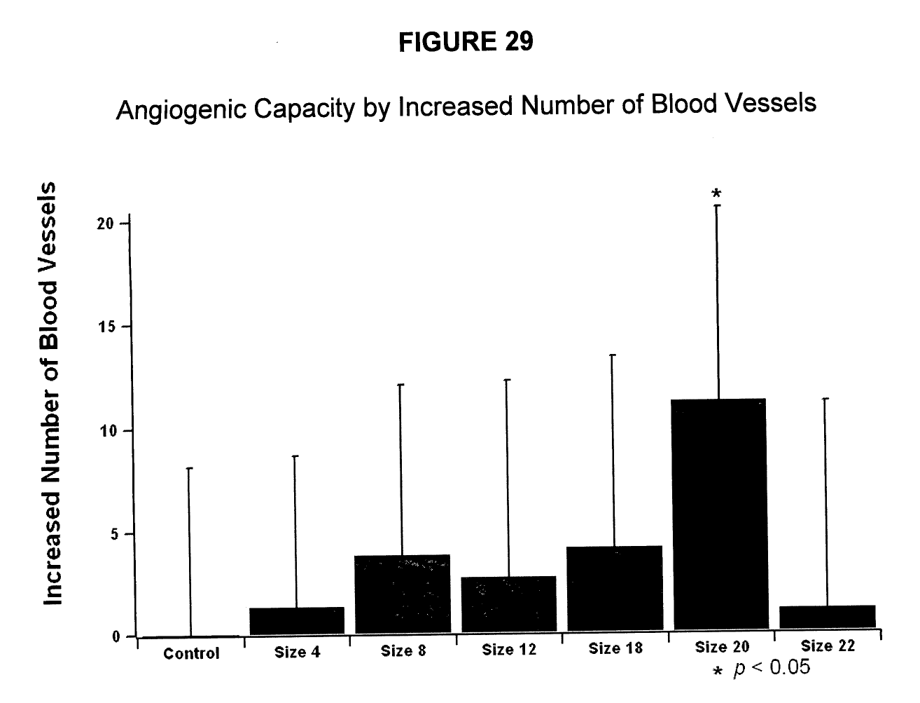

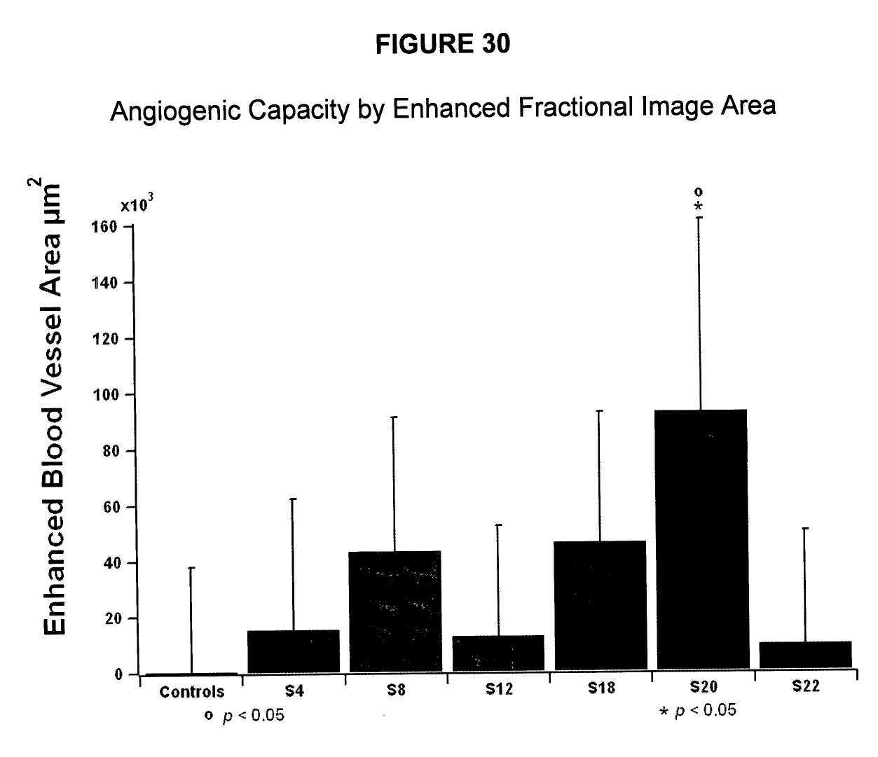

The avian chorioallantoic membrane (CAM) is a useful model to study angiogenesis and its regulation in vivo (Ribatti et al., 1996). Even though this model is based on avian systems, thus phylogenetically distant from mammals, it has been proven to be one of the most frequently successfully used models. Briefly, the HA oligosaccharides were applied to the CAM, the eggs were incubated for several days, and the blood vessel growth was monitored by light microscopy. The HA samples were compared to water as negative controls. The number of vessels (FIG. 29) or the area the vessels encompassed (FIG. 30) were measured. HA20 was the optimal size in this standard assay. Similar testing of various HA oligos in various models for other tissues would yield the best HA molecule for treating the condition of that model.

Tables VI and VII list the effects of different size HA on cell behavior and physiology. These tables clearly demonstrate the importance of HA size in treating certain conditions, as one HA size may cause one biological result, while another HA size may cause the exact opposite biological result in another system. In addition, it is also evident from these tables that a single HA size range may cause one biological result in one cell type (i.e., one type of cancer) and the opposite biological result in another cell type (i.e., another type of cancer or a healthy cell). For example, an HA size of 10³ causes increased metastasis in human chondrosarcoma cells and decreased metastasis in mouse mammary carcinoma, human colon carcinoma, and rat glioma cells. These results clearly demonstrate the need for the “personalized medicine” approach of the present invention, in which customized defined, specific GAG molecules are administered to a patient, wherein the defined, specific GAG molecules are chosen based on the specific ailment from which the patient is suffering and/or the response of in vitro testing of the ability of the defined, specific GAG molecules to treat, inhibit and/or prevent the ailment in a sample (i.e., biopsy) from the patient.

One strategy for patient treatment according to the methods of the presently disclosed and claimed invention would include the harvest and use of a sample from a patient (such as a biopsy or tissue) in an in vitro test to monitor reduction of a disease state (e.g., the cancer state or the modulation of angiogenesis). This test may be performed by contacting the patient sample with various sizes of GAGs and various compositions of GAGs, and assessing the optimal effective size and composition of GAG based on the consideration for healthy tissue effects. Alternatively, the GAG may be in a probe state (i.e., radioactive, fluorescent, NMR-active or other state disclosed herein or known in the art) and/or medicant state which is administered for localization and/or treatment of diseased tissue for potential subsequent or concurrent surgical, radiological or chemical modalities.

TABLE VI
Effects of different size HA on cell behavior and physiology (in vitro incubation)

| | Biological | HA Size | | |
|---|---|---|---|---|
| Effect | Result | (Daltons) | Cell Type | References |
| Induces angiogenesis | | 800-5000 | chick chorioallantoic | West et al., 1985 |
| | | | membrane | |
| Induces angiogenesis and cell proliferation | | 600-3200 | bovine endothelial cells | West et al., 1989 |
| Induces expression of IL-1β, TNF-α, and IGF- | increased | 4-8 × 10⁴ | mouse bone marrow- | Noble et al., 1993 |
| 1 by a TNF-α-dependent mechanism | inflammation | | derived macrophages | |
| Stimulates angiogenesis | | 1350-4500 | in vivo incubation on rat | Sattar et al., 1994 |
| | | | backs | |
| Stimulates cell migration | | 1350-4500 | bovine aortic endothelial | Sattar et al., 1994 |
| | | | cells | |
| Induces angiogenesis | | 1000-4000 | cryoinjured skin grafts | Lees et al., 1995 |
| Activates NF-κB/I-κB system | increased | <5 × 10⁵ | mouse alveolar | Noble et al., 1993 |
| | inflammation | | macrophages | |
| Induces expression of the chemokines | increased | <5 × 10⁵ | mouse alveolar | McKee et al., 1996 |
| RANTES, MIP-1α & β, and crg-2 and the | inflammation | | macropages and human | |
| cytokine IL-8 by a CD44-dependent | | | monocytic leukemia | |
| mechanism | | | cells | |
| Induces expression of iNOS in synergy with | increased | ˜2 × 10⁵ | mouse alveolar and | McKee et al., 1997 |
| IFN-γ by a NF-κB-dependent mechanism | inflammation | | bone marrow-derived | |
| | | | macrophages | |
| Induces expression of early-response genes | increased | 1350-4500 | bovine aortic endothelial | Deed et al., 1997 |
| like c-fos and c-jun (essential for cell | angiogenesis | | cells | |
| proliferation) | | | | |
| Induces expression of the chemokines | increased | ˜2.8 × 10⁵ | thioglycollate-elicited | Hodge-Dufour et |
| RANTES and MIP-1α & β, and the cytokine | inflammation | | mouse macrophages | al., 1997 |
| IL-12 by a CD44-dependent mechanism | | | | |
| Inhibits tumor growth | | 1200-4800 | mouse melanoma cells | Zeng et al., 1998 |
| Induces cell proliferation through a pathway | increased | 1350-4500 | bovine aortic endothelial | Slevin et al., 1998 |
| involving the phosphorylation of CD-44 and | angiogenesis | | cells | |
| the activation of PKC | | | | |
| Increases expression of ICAM-1 and VCAM- | increased | 0.8-6 × 10⁵ | mouse cortical tubular | Oertli et al., 1998 |
| 1 by a NF-κB-dependent mechanism | inflammation | | cells | |
| IL-10 and IFN-γ inhibit HA-induced | increased | ˜2 × 10⁵ | mouse bone marrow- | Horton et al., 1998 |
| expression of MIP-1α, MIP-1β, and KC | inflammation | | derived and | |
| | | | thioglycollate-elicited | |
| | | | peritoneal macrophages | |
| Induces expression of iNOS in synergy with | increased | ˜2 × 10⁵ | rat hepatocytes, | Rockey et al., 1998 |
| IFN-γ by a NF-κB-dependent mechanism | inflammation | | endothelial, Kupffer, | |
| | | | and stellate cells | |
| Induces expression of the chemokines Mig | increased | ˜2 × 10⁵ | mouse alveolar | Horton et al., 1998 |
| and IP-10 in synergy with IFN-γ by a TNF-α | inflammation | | macrophages | |
| independent mechanism | | | | |
| Stimulates MCP-1 production by a CD44- | localized | 0.8-8 × 10⁵ | SV40-transformed | Beck-Schimmer et |
| dependent mechanism | inflammation | | mouse cortical tubular | al., 1998 |
| | | | cells (renal epithelium) | |
| Induces expression of metalloproteinase | increased | ˜2 × 10⁵ | mouse and rat alveolar | Horton et al., 1999 |
| metalloelastase | inflammation | | macrophages | |
| Activates NF-κB signaling pathway by a | increased | ˜2 × 10⁵ | human bladder, | Fitzgerald et al., |
| CD44-dependent mechanism | inflammation | | cervical, and breast | 2000 |
| | | | carcinomas; mouse | |
| | | | macrophages | |
| Induces production of cytokines IL-1β, TNF- | cell | 800-1200 | human dendritic cells | Termeer et al., |
| α, and IL-12 and induces immunophenotypic | maturation, | | and mouse bone | 2000 |
| maturation of cells by a TNF-α-dependent | increased | | marrow—derived | |
| mechanism | inflammation | | macrophages | |
| Stimulates the mitogenic response and | increased | 4000-6000 | human pulmonary and | Lokeshwar et al., |
| protein tyrosine phosphorylation | angiogenesis | | lung microvessel | 2000 |
| | | | endothelial cells | |
| Stimulates expression of ICAM-1, TGF-β, and | increased | ˜2 × 10⁵ | peripheral blood | Ohkawara et al., |
| GM-CSF by a CD44-dependent mechanism | inflammation | | eosinophils | 2000 |
| and improves survival and changes | | | | |
| morphology of cells | | | | |
| Prevents liver injury caused by TNF-α | decreased | 4.5-9 × 10⁴ | mouse (in vivo) | Wolf et al., 2001 |
| | inflammation | | | |
| Induces maturation of dendritic cells via the | increased | 800-1200 | human dendritic cells | Termeer et al., |
| Toll-like receptor-4 by a NF-κB-dependent | inflammation | | and mouse bone | 2002 |
| mechanism | | | marrow—derived | |
| | | | macrophages | |
| Stimulates expression and tyrosine | increased | ˜3.5 × 10³ | human chondrosarcmoa | Suzuki et al., 2002 |
| phosphorylation of c-Met, the hepatocyte | metastasis | | cells | |
| growth/scatter factor receptor, by a CD44- | | | | |
| dependent mechanism | | | | |
| Induces cell proliferation, wound recovery, | increased | 1350-4500 | bovine aortic endothelial | Slevin et al., 2002 |
| and activation of ERK 1/2 through a pathway | angiogenesis | | cells | |
| involving Ras and Src and induces | | | | |
| angiogenesis using G-proteins, PLCγ1, and | | | | |
| PKC | | | | |
| Induces tyrosine phosphorylation and | decreased | ˜3.2 × 10⁴ | human lung cancer cells | Fujita et al., 2002 |
| activation of focal adhesion kinase which then | apoptosis | | transfected with CD44 | |
| associates with PI 3-kinase and activates | | | | |
| mitogen-activated protein kinase | | | | |
| Inhibits tumor growth and promotes apoptosis | decreased | ˜2.5 × 10³ | mouse mammary and | Ghatak et al., |
| by suppressing the PI 3-kinase/Akt cell | metastasis | | human colon carcinoma | 2002 |
| survival pathway | | | cells | |
| Induces expression of Mig in synergy with | increased | ˜2 × 10⁵ | mouse alveolar | Horton et al., 2002 |
| IFN-γ by a NF-κB-dependent mechanism | inflammation | | macrophages | |
| Stimulates expression of urokinase-type | increased | ˜3.5 × 10³ | human chondrosarcoma | Kobayashi et al., |
| plasminogen activator and its receptor, | metastasis | | cells | 2002 |
| phosphorylation of MAP kinase proteins, and | | | | |
| cell invasion by a CD44-dependent | | | | |
| mechanism | | | | |
| Stimulates proliferation and haptotactic | increased | | malignant mesotheioma | Nasreen et al., |
| migration by a CD44-dependent mechanism | metastasis | | cells | 2002 |
| Protects from damage by oxygen free radicals | antioxidative | | rat wounds | Trabucchi et al., |
| | | | | 2002 |
| Stimulates cell growth and increases | stimulation of | 6 × 10⁴ | rat mesenchymal cells | Huang et al., 2003 |
| osteocalcin expression | osteoblasts | | | |
| Sensitizes tumor cells to chemotherapeutic | decreased | ˜2.5 × 10³ | human mammary | Misra et al., 2003 |
| drugs by suppressing the MAP kinase and PI | drug | | carcinoma cells | |
| 3-kinase pathways | resistance | | | |
| Induces cleavage of CD44 and promotes cell | increased | <3.6 × 10⁴ | human pancreatic | Sugahara et al., |
| motility | metastasis | | carcinoma cells | 2003 |
| Inhibits endogenous HA polymer interaction, | decreased | ˜2.5 × 10³ | rat glioma cells | Ward et al., 2003 |
| thus reducing HA-induced signaling | metastasis | | | |
| Increases production of IL-8 | increased | ˜2 × 10⁵ | human lung fiobroblasts | Bai et al., 2005 & |
| | inflammation | | | Mascarenhas et |
| | | | | al., 2004 |
| Increases production of IL-8 by Toll-like | increased | 800-1600 | human endothelial cells | Taylor et al., 2004 |
| receptor-4-dependent mechanism | inflammation | | | |
| Induces chondrolysis by upregulating | increased | 1200 | bovine articular | Ohno et al., 2005 |
| pathways involved in cartilage remodeling | catabolism | | chondrocytes | & Knudson et al., |
| | | | | 2000 |

TABLE VII
Effects of Different Size HA on cell behavior and physiology (tissue culture)

| | HA Size | | | |
|---|---|---|---|---|
| Effect | (Daltons) | Cell Type | Method | References |
| Inhibits phagocytosis | 0.46-2.8 × 10⁵ | mouse peritoneal | phagocytosis | Forrester et al., 1980 |
| | | macrophage | of latex | |
| | | | spheres | |
| Inhibits cell proliferation | >10⁶ | Bovine endothelial | in vitro | West et al., 1989 |
| | | cells | incubation | |
| Inhibits cells proliferation | >10⁶ | bovine aortic | in vitro | West et al., 1991 |
| | | endothelial cells | incubation | |
| Provides structure and elasticity in | >10⁶ | | | Laurent et al., 1996 |
| synovial fluid | | | | |
| Inhibits induction of early-response | >10⁶ | bovine aortic | in vitro | Deed et al., 1997 |
| gene expression | | endothelial cells | incubation | |
| Inhibits HA fragment stimulation of | >10⁶ | SV40-transformed | in vitro | Beck-Schimmer et al., 1998 |
| MCP-1 production | | mouse cortical tubular | incubation | |
| | | cells (renal epithelium) | | |
| Reduces contact inhibition of growth | | | | Itano et al. 2002 |
| and promotes migration | | | | |
| Mediates and modulates cell-matrix | 2.7 × 10⁶ | frog kidney epitelial | cell | Zimmerman et al., 2002 |
| adhesion | | cells | attachment to | |
| | | | HA-coated | |
| | | | crystals | |
| Inhibits cell migration by down- | Sigma | human preosteoclast | in vitro | Spessotto et al., 2002 |
| regulating the expression of the | | cells | incubation | |
| metalloproteinase MMP-9 in a CD44- | | | | |
| dependent mechanism | | | | |
| Enhanced the IL-2-induced edema | Sigma | lung and liver | in vivo | Mustafa et al., 2002 |
| and lymphocytic infiltration | (5-8 × 10⁶) | | administration | |
| Decreases and/or repairs damage to | 8 × 10⁵ | bovine and human | in vitro | Homandberg et al., 2003 & |
| proteoglycan caused by fibronectin | | cartilage | incubation | Williams et al., 2003 |
| fragments | | | | |
| Restores the attachment and | 9.5 × 10⁵ | bovine chondrocytes | in vitro | Kim et al., 2003 |
| migration of chondrocytes suppressed | | | incubation | |
| by IL-1α | | | | |
| Induces drug resistance and | HAS2 | human mammary | in vivo | Misra et al., 2003 & Marieb |
| anchorage-independent growth. | | carcinoma cells | expression | et al., 2004 |
| Increased production due to elevated | | | | |
| emmprin expression stimulates cell | | | | |
| survival pathway signaling. | | | | |
| Induces osteoblast differentiation and | 0.9-2.3 × 10⁶ | rat mesenchymal cells | in vitro | Huang et al., 2003 |
| bone formation | | | incubation | |
| Increases cell viability and survival | 5-7 × 10⁵ | human chondrocytes | in vitro | Brun et al., 2003 |
| after oxidative cell injury, both in a | | | incubation | |
| CD44-dependent mechanism | | | | |
| Regulates localization, proliferation, | 0.2-1 × 10⁵ | mouse and human | in vivo | Nilsson et al., 2003 |
| and differentiation | | hemopoietic stem | expression | |
| | | cells | | |
| Prevents perineural scar formation | Orthovisc | rat nerve cells | in vivo | Ozgenel, 2003 |
| and enhances peripheral nerve | | | administration | |
| regeneration | | | | |
| Promotes adhesion to laminin, | HAS2&3 | human colon | in vivo | Laurich et al., 2004 |
| facilitating invasion and metastasis | | carcinoma cells | expression | |
| Promotes hypertrophic changes; | HAS2 | rabbit chondrocytes | in vivo | Suzuki et al., 2005 |
| modulates and maintains cartilage | | | expression | |
| Prevents liver injury by reducing | ≧7.8 × 10⁵ | rat liver cells | in vivo | Nakamura et al., 2004 |
| proinflammatory cytokines | | | administration | |
| Exhibits antioxidative effects | ≧2.2 × 10⁵ | lipid model system | in vitro | Trommer et al., 2003 |
| | | | incubation | |
| Decreased dexamethasone-induced | Sigma | human malignant | in vitro | Vincent et al., 2003 |
| apoptosis | | multiple myeloma | incubation | |
| | | cells | | |
| Inhibits cell proliferation | ˜1 × 10⁶ | rat primary cortical | in vitro | Struve et al., 2005 |
| | | astrocytes | incubation | |
| Promotes tumor growth, metastasis, | | | | Liu et al., 2001; Kosaki et al., |
| and/or angiogenesis | | | | 1999; Itano et al., 1999; |
| | | | | Ichikawa et al., 1999; |
| | | | | Simpson et al., 2002; |
| | | | | Jacobson et al., 2002; and |
| | | | | Jojovic et al., 2002 |

### Materials and Methods

Methods were performed as described in parent application U.S. Ser. No. 10/642,248, which has previously been incorporated herein by reference, except as described herein below.

Acceptor Preparation—All reagents were the highest grade available from either Sigma or Fisher unless otherwise noted. The tetrasaccharide HA4, the starting acceptor for the synthesis of longer polymers, was generated by exhaustive degradation of streptococcal HA polymer with ovine testicular hyaluronidase Type V and purified by extensive chloroform extraction, ultrafiltration, and size exclusion chromatography. The HA4 molecule was converted into a fluorescent derivative in two steps. First, an amino-HA4 derivative was prepared by reductive amination of HA4 (12 mM) with sodium cyanoborohydride (70 mM) and excess diaminoethane (200 mM) in 0.1 M borate buffer, pH 8.5, 1 mM CuCl₂ at 37° C. for 2 days. The amino-HA4 product was purified on P2 resin. Second, a fluorescent acceptor was prepared by derivatizing amino-HA4 with the N-hydroxysuccinimide ester of Oregon Green™ 488 (3-fold molar excess; Molecular Probes, Eugene, Oreg.) in 50% dimethylsulfoxide, 100 mM Hepes buffer, pH 8.5. The major isomer of fluor-HA4 was purified by preparative normal-phase thin layer chromatography (2:1:1 n-butanol/acetic acid/water and silica, Whatman). The identities of HA4, amino-HA4, and fluor-HA4 were verified by virtue of the agreement of their expected and experimental masses (775 Da, 819 Da, and 1213 Da, respectively) as assessed by matrix-assisted laser desorption ionization time-of-flight mass spectrometry in negative mode (DeAngelis et al., 2003).

Catalyst preparation and in vitro synthesis—The catalysts, pmHAS¹⁻⁷⁰³, and pmCS¹⁻⁷⁰⁴, are soluble purified Escherichia coli-derived recombinant proteins (Jing et al., 2000). The enzymes in the octyl-thioglucoside cell extracts were purified by chromatography on Toyopearl Red AF resin (Tosoh) using salt elution (50 mM HEPES, pH 7.2, 1 M ethylene glycol (an enzyme stabilizer) with 0 to 1.5 M NaCl gradient in 1 hour) (DeAngelis et al., 2003). The fractions containing the target protein (≧90% pure by SDS-PAGE/Coomassie-staining) were concentrated and exchanged into 1 M ethylene glycol, 50 mM Tris, pH 7.2, by ultrafiltration with an Amicon spin unit (Millipore). The selectHA monodispserse syntheses in general contained pmHAS¹⁻⁷⁰³, UDP-GlcNAc, UDP-GlcUA, 5 mM MnCl₂, 1 M ethylene glycol, 50 mM Tris, pH 7.2, and a sugar acceptor. Reactions were incubated at 30° C. for 2 to 72 hrs. The soluble, truncated dual-action wild-type pmHAS¹⁻⁷⁰³ enzyme was mutated with the QuickChange system (Stratagene) to produce a pair of single-action enzymes: the GlcNAc-Tase pmHAS¹⁻⁷⁰³ (D527N, D529N) and the GlcUA-Tase pmHAS¹⁻⁷⁰³ (D247N, D249N). The mutant enzymes in the bacterial lysates (Jing et al., 2000) were purified by chromatography on Toyopearl Red AF resin (Tosoh), and the fractions containing the mutant protein were immobilized via their free amino groups to N-hydroxysuccinimide agarose beads (Sigma). Typically, ˜95% of the protein was coupled to the beads after mixing for 4-6 hours at 4° C. Residual activated esters were quenched with 50 mM Tris, pH 7.2, 1 M ethylene glycol buffer (TEG) for 2 hours at 4° C. before washing the beads extensively with more TEG. The enzyme reactors (˜18 mg protein on 4 ml of packed beads in a small glass column) were catalytically active for at least 8 months with storage at 4° C. in TEG buffer with 0.05% sodium azide preservative.

Analysis of in vitro synthesized HA—The size of HA was analyzed on agarose gels (0.7-1.2%; 1×TAE buffer (40 mM Tris acetate, 2 mM EDTA); 40V) stained with Stains-All dye (0.005% w/v in ethanol) (Lee et al., 1994). Approximately 0.5-5 μg of HA was loaded per lane. For smaller HA polymers (<40 kDa), HA was also analyzed on polyacrylamide gels (15-20%) with acridine orange staining (Ikegami-Kawai et al., 2002). To purify HA for later analysis, pmHAS was removed by chloroform extraction and the HA product was precipitated with three volumes of ethanol and the pellets were redissolved in water. Alternatively, the unincorporated precursor sugars were removed by ultrafiltration (Microcon units, Millipore). The HA concentration was determined by the carbazole assay using a glucuronic acid standard (Bitter et al., 1962).

Size exclusion chromatography/multi-angle laser light scattering (SEC-MALLS) analysis was employed to determine the absolute molecular masses of HA products. Polymers (2.5 to 12 μg mass; 50 μl injection) were separated on Polymer Laboratories PL aquagel-OH 30 (8 μm), —OH 40, —OH 50, —OH 60 (15 μm) columns (7.5×300 mm, Polymer Laboratories, Amherst, Mass.) in tandem or alone as required by the size range of the polymers to be analyzed. The columns were eluted with 50 mM sodium phosphate, 150 mM NaCl, pH 7 at 0.5 ml/min. MALLS analysis of the eluant was performed by a DAWN DSP Laser Photometer in series with an OPTILAB DSP Interferometric Refractometer (632.8 nm; Wyatt Technology, Santa Barbara, Calif.). The ASTRA software package was used to determine the absolute average molecular mass using a dn/dc coefficient of 0.153 determined by Wyatt Technology. The Mw and polydispersity values are the average of data from at least two SEC-MALLS runs.

Chemoenzymatic Synthesis—In the typical oligosaccharide synthesis, 90 μmoles of acceptor oligosaccharide and 110-135 μmoles (1.2 to 1.5 equivalents) of UDP-sugar (˜15 mM final) in reaction buffer (TEG plus 17 mM MnCl₂) were circulated over an enzyme reactor at room temperature. The tetrasaccharide HA4, the starting acceptor for the synthesis of longer oligosaccharides, was generated by exhaustive degradation of streptococcal HA polymer (Sigma) with ovine testicular hyaluronidase Type V (Sigma) and purified by extensive chloroform extraction, ultrafiltration, and gel filtration chromatography on P2 (BioRad) resin. For converting HA4 starting material (with a GlcUA at the nonreducing terminus) into the pentasaccharide HAS, the GlcNAc from UDP-GlcNAc was transferred with the GlcNAc-Tase reactor.

The reactions were monitored by TLC (silica plates developed with n-butanol/acetic acid/H₂O, 1.5:1:1 for HA4 to HA8 or 1:1:1 for HA8 to HA14) and napthoresorcinol staining (dipped in 0.2% w/v reagent in 96% ethanol/4% sulfuric acid, followed by heating at 100° C.). Typically, each step of the 90-μmole scale reactions were judged to be complete by TLC within 1 or 2 passes of the mixture through the reactor (˜5 to 10 min contact time), but the reaction mixture was further recirculated for a total of 12 passes (˜1 to 2 hours) to insure virtually complete oligosaccharide conversion. After the reaction mixture was harvested, the enzyme reactor was washed with a column volume of TEG buffer and this washing was added to the reaction mixture. A small amount of MnCl₂ was added to compensate for the volume increase due to the wash step (final 17 mM).

The next UDP-sugar (in this specific case, UDP-GlcUA) was added to the reaction mixture and then applied to the next reactor (converting HA5 into the hexasaccharide HA6 with immobilized GlcUA-Tase). This repetitive synthesis was continued by adding the next appropriate UDP-sugar and switching enzyme reactors. Between each step, the reactors were washed extensively with TEG to remove any residual reaction products retained on the column from the previous step.

At the end of the desired synthesis, the reaction mixtures were lyophilized and the oligosaccharides were desalted by gel filtration on P4 (BioRad) resin eluted with 0.2 M ammonium formate. The major sugar peak was harvested and the volatile residual salts were removed by lyophilization from water three times.

HA20 was prepared starting with purified HA13 from the synthesis above. In this synthesis, for proof of principle and for convenience, all of the required UDP-sugars for the complete synthesis were added at the first step.

Oligosaccharide Analyses—For MALDI-TOF MS, the matrix solution (50 mg/ml 6-aza-2-thiothymine in 50% acetonitrile, 49.9% water, 0.1% trifluoroacetic acid, 10 mM ammonium citrate) was mixed 1:1 with the samples containing ˜0.1 μg/μl oligosaccharide in water, spotted onto the target plate, and vacuum dried. The samples were analyzed in the negative ion, reflectron mode on a Voyager Elite DE mass spectrometer (20 kV acceleration, low mass gate 800 Da, delayed extraction 180 ns). The oligosaccharides were also analyzed by 20% polyacrylamide gel electrophoresis with acridine orange staining as described previously (Ikegami-Kawai et al., 2002).

Soft agar assays were performed as described in Chapter 5, Growth Interactions in Cancer Metastasis, of Laboratory Techniques in Biochemistry and Molecular Biology (2000; Pillai and Van Der Vliet, eds.), and as described in Hamburger et al. (1980), all of which are incorporated herein by reference.

The chick embryo chorioallantoic membrane assays were performed as described in Chapter 9, Angiogenesis and Metastasis, of Laboratory Techniques in Biochemistry and Molecular Biology (2000; Pillai and Van Der Vliet, eds.), and as described in Ribatti et al. (1996) and Ribatti et al. (1997), all of which are incorporated herein by reference.

Although the foregoing invention has been described in detail by way of illustration and example for purposes of clarity of understanding, it will be obvious to those skilled in the art that certain changes and modifications may be practiced without departing from the spirit and scope thereof, as described in this specification and as defined in the appended claims below.

### REFERENCES

The following references, to the extent that they provide exemplary procedural or other details supplementary to those set forth herein, are specifically incorporated herein by reference in their entirety as though set forth herein particular.
Asplund, T., et al. (1998) Biochim. Biophys. Acta. 1380, 377-388.
Ausubel, F. M., et al. (1995) Short Protocols in Molecular Biology, 3^{rd} Ed., John Wiley & Sons Inc., New York.
Bai, K. J., et al. (2005) Am J Respir Crit Care Med.
Beck-Schimmer, B., et al. (1998) J Am Soc Nephrol 1998. 9(12): p. 2283-90.
Bello, Y M, et al. (2001) Am J Clin Dermatol, 2:305-313.
Bertram, J., et al. (1991) J. Bact. 173: 443-8.
Bitter, T. and H. M. Muir. (1962) Anal. Biochem. 4, 330-334.
Bradford, M. M., (1976) Anal. Biochem. 72, 248-254.
Breton, C. and A. Imberty. (1999) Curr. Opin. Struc. Biol. 9, 563-571.
Brun, P., et al. (2003) Osteoarthritis Cartilage, 11(3): p. 208-16.
Busch, C., et al. (1998) J. Biol. Chem., 273, 19566-19572.
Campbell, R. E., et al. (2000) Biochemistry 39, 7012-7023.
Carter, G. R. and E. Annau. (1953) Am. J. Vet. Res. 14, 475478.
Charnock, S. J. and G. J. Davies. (1999) Biochemistry, 38, 6380-6385.
Chen, W Y. and Abstangelo G. (1999) Wound Repair Regen, 7, 79-89.
Chung, J. Y., et al. (2001) Infect. Immun., 69, 2487-2492.
Corpet, F. (1998) Nucleic Acids Res. 16, 10881-10890.
Crater, D. L., and I. van de Rijn. (1995) J. Biol. Chem. 270, 18452-18458.
Day, A. J., and Prestwich, G. D. (2002) J Biol Chem 277, 4585-4588
DeAngelis, P. L., et al. (1993) J. Biol. Chem., 268, 19181-19184.
DeAngelis, P. L., et al. (1993) J. Biol. Chem., 268, 14568-14571.
DeAngelis, P. L. and P. H. Weigel. (1994) Biochemistry, 33, 9033-9039.
DeAngelis, P. L., and A. M. Achyuthan. (1996) J. Biol. Chem. 271(39):23657-60.
DeAngelis, P. L. (1996) Biochemistry. 35(30):9768-71.
DeAngelis, P. L., et al. (1997) Science. 278(5344):1800-3.
DeAngelis P. L. (1998) Microb. Pathog. 24(4):203-9.
DeAngelis, P. L., et al. (1998) J. Biol. Chem. 273(14):8454-8.
DeAngelis, P. L. (1999) J. Biol. Chem. 274(37); 26557-62.
DeAngelis, P. L. (1999) Cell. Mol. Life Sci., 56, 670-682.
DeAngelis, P. L. (2000) Anal. Biochem. 284(1):167-9.
DeAngelis, P. L. and A. J. Padgett-McCue. (2000) J. Biol. Chem. 275(31):24124-9.
DeAngelis, P. L. (2002) Glycobiology. 12(1):9R-16R. Review.
DeAngelis, P. L., and C. L. White. (2002) J. Biol. Chem. 277(9):7209-13.
DeAngelis, P. L., et al. (2003) J. Biol. Chem., 278, 35199-35203.
DeAngelis, P. L., M. H. Graves, and J. L. Van Etten, unpublished results.
DeLuca, S. and J. E. Silbert. (1968) J. Biol. Chem. 243, 2725-2729.
Deed, R., et al. (1997) Int J Cancer. 71(2): p. 251-6.
Doughtery, B. A., and I. van de Rijn. (1994) J. Biol. Chem., 269, 169-175.
Drake, C. R., et al. (1990) FEMS Microbiol. Lett., 54, 227-230.
Duncan, G., et al. (2001) J. Clin. Invest., 108, 511-516.
Esko, J. D. and U. Lindahl. (2001) J. Clin. Invest. 108,169-173.
Fieber, C., et al. (2004) J Cell Sci, 117, 359-367
Finke, A., et al. (1991) J. Bacteriol., 173, 40884094.
Fitzgerald, K. A., et al. (2000) J Immunol, 164(4): p. 2053-63.
Forrester, J. V. and E. A. Balazs. (1980) Immunology, 40(3): p. 43546.
Fujita, Y., et al. (2002) FEBS Lett, 528(1-3): p. 101.
Gastinel, L. N., et al. (1999) EMBO J. 18, 3546-3557.
Gastinel, L. N., et al. (2001) EMBO J. 20, 638-649.
Ghatak, S., et al. (2002) J Biol Chem, 277(41): p. 38013-20.
Gherezghiher, T., et al. (1987) J. Chromatogr. 413, 9-15.
Gietz, R. D., et al. (1995) Yeast, 11, 355-360.
Griffiths, G., et al. (1998) J. Biol. Chem., 273, 11752-11757.
Hagopian, A. and E. H. Eylar. (1968) Arch. Biochim. Biophys., 128, 422433.
Halkes, K. M., et al. (1998) Carbohydr Res, 309, 161-174.
Hall, N. A. and A. D. Patrick. (1989) Anal. Biochem. 178, 378-384.
Hamburger, A. W., et al. (1980) Prog. Clin. Biol. Res. 48:43-52.
Hansen, L. M. and D. C. Hirch. (1989) Vet. Microbiol. 21, 177-184.
Hardingham, T. E. and A. J. Fosang. (1992) FASEB J. 6, 861-870.
Harmon, B. G., et al. (1991) Am. J. Vet. Res. 52, 1507-1511.
Hascall, V. C. and G. K. Hascall. (1981) in Cell Biology of Extracellular Matrix (Hay, E. D., ed) pp. 39-78, Plenum Publishing Corp. New York.
Heldermon, C., et al. (2001) J. Biol. Chem., 276, 2037-2046.
Hempel, J., et al. (1994) Protein Sci. 3, 1074-1080.
Hodge-Dufour, J., et al. (1997) J Immunol, 159(5): p. 2492-500.
Hodson, N., et al. (2000) J. Biol. Chem., 275, 27311-27315.
Hofmann, K. and W. Stoffel. (1993) J. Biol. Chem. 347, 166 (abstr.)
Iivanainen, E., et al. (2003) Microsc Res Tech, 60:13-22.
Homandberg, G. A., et al. (2003) Osteoarthritis Cartilage, 11(3): p. 177-86.
Horton, M. R., et al. (1998) J Immunol, 160(6): p. 3023-30.
Horton, M. R., et al. (1998) J Biol Chem, 273(52): p. 35088-94.
Horton, M. R., et al. (1999) J Immunol, 162(7): p. 4171-6.
Horton, M. R., et al. (2002) J Biol Chem, 277(46): p. 43757-62.
Huang, L., et al. (2003) J Biomed Mater Res A, 66(4): p. 880-4.
Ichikawa, T., et al. (1999) J Invest Dermatol, 113(6): p. 935-9.
Ikegami-Kawai, M., and Takahashi, T. (2002) Anal Biochem, 311, 157-165.
Isacke, C. M., and Yarwood, H. (2002) Int J Biochem Cell Biol, 34, 718-721.
Itano, N., et al. (1999) J. Biol. Chem. 274, 25085-25092.
Itano, N., et al. (1999) Cancer Res, 59(10): 2499-504.
Itano, N., et al. (2002) Proc Natl Acad Sci USA, 99(6):3609-14.
Itano, N., and Kimata, K. (2002) IUBMB Life, 54,195-199.
Jacobson, A., et al. (2002) Int J Cancer, 102(3):212-9.
Jing, W., and DeAngelis, P. L. (2000) Glycobiology, 10(9):883-889.
Jing, W., and DeAngelis, P. L. (2003) Glycobiology, 13:661R-671R.
Jojovic, M., et al. (2002) Cancer Lett, 188(1-2):181-9.
Kim, G., et al. (2003) J Vet Med Sci, 65(3):427-30.
Kitagawa, H., et al. (2001) J. Biol. Chem., 276, 38721-38726.
Knudson, C. B. and W. Knudson (1993) FASEB. J. 7, 1233-1241.
Knudson, W., et al. (2000) Arthritis Rheum, 43(5):1165-74.
Kobayashi, H., et al. (2002) Int J Cancer, 102(4):379-89.
Koizumi, S., et al. (1998) Nat Biotechnol, 16, 847-850.
Kosaki, R., et al. (1999) Cancer Res, 59(5):1141-5.
Koyama, M., et al. (1997) Proc. Natl. Acad. Sci. U.S.A. 94, 9091-9095.
Kroll, J. S., et al. (1990) Mol. Microbiol. 4, 1853-1862.
Kumari, K. and P. H. Weigel. (1997) J. Biol. Chem., 272, 32539-32546.
Laurent, T. C., and Fraser, J. R. (1992) Faseb J, 6, 2397-2404.
Laurent, T. C., et al. (1996) Immunol Cell Biol, 74(2): p. A1-7.
Laurich, C., et al. (2004) J Surg Res, 122(1):70-4.
Lee, C. J. (1987) Mol. Immunol., 24, 1005-1019.
Lee, H. G., and Cowman, M. K. (1994) Anal Biochem, 219, 278-287.
Lee, J. Y., and Spicer, A. P. (2000) Curr Opin Cell Biol, 12, 581-586.
Lees, V. C., et al. (1995) Lab Invest, 73(2):259-66.
Li, J., et al. (2001) Glycobiology, 11, 217-229.
Lidholt, K. and U. Lindahl. (1992) Biochem J. 287, 21-29.
Lidholt, K. (1997) Biochem. Soc. Trans. 25, 866-870.
Lidholt, K. and M. Fjelstad. (1997) J. Biol. Chem. 272, 2682-2687.
Lind, T., et al. (1993) J. Biol. Chem. 268, 20705-20708.
Lind, T., et al. (1998) J. Biol. Chem. 273, 11752-11757.
Lindahl, U. and M. Hook. (1978) Annu. Rev. Biochem. 47, 385-417.
Liu, N., et al. (2001) Cancer Res, 61(3):1022-8.
Lokeshwar, V. B. and M. G. Seizer. (2000) J Biol Chem, 275(36):27641-9.
Ludwigs, U., et al. (1987) Biochem. J., 245, 795-804.
Mahoney, D. J., et al. (2001) Glycobiology, 11, 1025-1033.
Makrydimas, G., et a. (2003) In vivo, 17(6):633-640.
Marhaba, R. And M. Zoller. (2004) J. Mol. Histol. 35(3):211-231.
Marieb, E. A., et al. (2004) Cancer Res, 64(4):1229-32.
Markovitz, A., et al. (1959) J. Biol. Chem. 234, 2343-2350.
Marks, D. L., et al. (2001) J. Biol. Chem. 276, 26492-26498.
Mascarenhas, M. M., et al. (2004) Am J Respir Cell Mol Biol, 30(1):51-60.
Maxwell, C. A., et al. (2004) Blood, 104(4):1151-1158.
May, B. J., et al. (2001) Proc. Natl. Acad. Sci. U.S.A. 98, 3460-3465.
McKee, C. M., et al. (1996) J Clin Invest 98, 2403-2413.
McKee, C. M., et al. (1997) J Biol Chem, 272(12):8013-8.
Meyer, M. F., and G. Kreil (1996) Proc. Natl. Acad. Sci. U.S.A. 93, 4543-4547.
Misra, S., et al. (2003) J Biol Chem, 278(28):25285-8.
Morera, S., et al. (1999) J. Mol. Biol. 311, 569-577.
Morera, S., et al. (2001) J. Mol. Biol., 311, 569-577.
Mustafa, A., et al. (2002) J Immunother, 25(6):476-88.
Nakamura, K., et al. (2004) J Gastroenterol, 39(4):346-54.
Nasreen, N., et al. (2002) Oncol Res, 13(2):71-8.
Nilsson, S. K., et al. (2003) Blood, 101(3):856-62.
Noble, P. W., et al. (1996) J Exp Med, 183(5):2373-8.
Noble, P. W., et al. (1993) J Clin Invest, 91(6):2368-77.
Oertli, B., et al. (1998) J Immunol, 161(7):3431-7.
Ohkawara, Y., et al. (2000) Am J Respir Cell Mol Biol, 23(4):444-51.
Ohno, S., et al. (2005) Arthritis Rheum, 52(3):800-9.
Ohya, T. and Y. Kaneko. (1970) Biochim. Biophys. Acta 198, 607-609.
Ozgenel, G. Y. (2003) Microsurgery, 23(6):575-81.
Pedersen, L. C., et al. (2000) J. Biol. Chem., 275, 34580-34585.
Persson, K., et al. (2001) Nat. Struct. Biol 8, 166-175.
Petit, C., et al. (1995) Mol. Microbiol., 17, 611-620.
Prehm, P. (1983) Biochem. J. 211, 181-189.
Prehm, P. (1983) Biochem. J. 211, 191-198.
Pummill, P. E., and P. L. DeAngelis. (2002) J. Biol. Chem. 277(24):21610-6.
Pummill P. E., et al. (1998) J. Biol. Chem. 273(9):4976-81.
Quinn, A. W., and K. P. Sing. (1957) Proc. Soc. Exp. Biol. Med. 95, 290-294.
Radominska, A. and R. R. Drake. (1994) Methods Enzymol. 230, 330-339.
Rahemtulla, F. and S. Lovtrup. (1975) Comp. Biochem. Physiol. 50B, 631-635.
Ramakrishnan, B. and P. Qasba. (2001) J. Mol. Biol. 310, 205-218.
Ribatti, D., et al. (1997) J. Vasc. Res. 34:455-463.
Ribatti, D., et al. (1996) Int. J. Dev. Biol. 40:1189-1197.
Rimler, R. B. (1994) Vet Rec. 134,191-192.
Rimler, R. B. and K. R. Rhodes. (1987) J. Clin. Microbiol. 25, 615-618.
Rimler, R. B. (1994) Vet. Rec. 134,191-192.
Rimler, R. B., et al. (1995) Vet. Microbiol. 47, 287-294.
Roberts, I. S., et al. (1988) J. Bacteriol. 170, 1305-1310.
Roberts, I. S. (1996) Annu. Rev. Microbiol. 50, 285-315.
Rockey, D. C., et al. (1998) Hepatology, 27(1):86-92.
Roden, L. (1980) in The Biochemistry of Glycoproteins and Proteoglycans (Lennarz, W. J., ed) pp. 267-371, Plenum Publishing Corp. New York.
Rodriguez, M. L, et al. (1988) Eur. J. Biochem. 177, 117-124.
Rohozinski, J., et al. (1989) Virology, 168:363.
Rosa, F., et al. (1988) Dev. Biol 129, 114-123.
Rosner, H., et al. (1992) Carbohydr. Res. 223, 329-333.
Sambrook, J., et al. (1989) Molecular Cloning: A Laboratory Manual, 2^{nd} edn. Cold Spring Harbor, N.Y.: Cold Spring Laboratory Press.
Sattar, A., et al. (1994) J Invest Dermatol, 103(4):576-9.
Saxena, I. M., et al. (1995) J. Bacteriol., 177: 1419-1424.
Semino, C. E. and P. W. Robbins. (1995) Proc. Natl. Acad. Sci. U.S.A. 92, 3498-3501.
Semino, C. E., et al. (1996) Proc. Natl. Acad. Sci. U.S.A. 93, 4548-4553.
Serra, M., et al. (2005) Int J Cancer, 114(6):879-886.
Simpson, M. A., et al. (2002) Am J Pathol, 161(3):849-57.
Slevin, M., et al. (1998) Lab Invest, 78(8): p. 987-1003.
Slevin, M., et al. (2002) J Biol Chem, 277:4104641059.
Soltes, L., et al. (2002) Biomed. Chromatogr. 16, 459462.
Song, H H. and Filmus, J. (2002) Biochim Biophys Acta, 1573:241-246.
Spessotto, P., et al. (2002) J Cell Biol, 158(6):1133-44.
Spicer, A. P., et al. (1998) J Biol Chem, 273:25117-25124.
Spicer, A. P. and J. A. McDonald. (1998) J. Biol. Chem. 273, 1923-1932.
Stoolmiller, A. C. and A. Dorfman. (1969) J. Biol. Chem. 244, 236-346.
Storgard, C., et al. (2005) Methods Mol Biol, 294:123-136.
Struve, J., et al. (2005) Glia.
Sugahara, K. N., et al. (2003) J Biol Chem, 278(34):32259-65.
Sugahara, K., et al. (1979) J. Biol. Chem. 254, 6252-6261.
Sunthankar, P. et al. (1998) Anal. Biochem., 258(2): 195-201.
Suzuki, M., et al. (2002) Biochim Biophys Acta, 1591(1-3):37-44.
Suzuki, A., et al. (2005) Biochim Biophys Acta, 1743(1-2):57-63.
Svanborg-Eden, C., et al. (2001) J. Mol. Biol. 314, 655-661.
Tawada, A., et al. (2002) Glycobiology, 12:421-426.
Taylor, K. A., and J. G. Buchanan-Smith. (1992) Anal. Biochem. 201, 190-196.
Taylor, K. R., et al. (2004) J Biol Chem, 279(17):17079-84.
Telser, A., et al. (1965) Proc. Natl. Acad. Sci. U.S.A. 54, 912-919.
Tengblad, A. (1980) Biochem. J. 185, 101-105.
Termeer, C. C., et al. (2000) J Immunol, 165(4):1863-70.
Termeer, C., et al. (2002) J Exp Med, 195(1):99-111.
Tlapak-Simmons, V. L., et al. (1998) J. Biol. Chem., 273, 26100-26109.
Tlapak-Simmons, V. L., et al. (1999) J. Biol. Chem. 274, 4246-4253.
Toole, B. P. (2001) Semin Cell Dev Biol, 12:79-87.
Toole, B. P. (2002) Glycobiology, 12:37R-42R.
Townsend, K. M., et al. (2001) J. Clin. Microbiol., 39:924-929.
Trabucchi, E., et al. (2002) Int J Tissue React, 24(2):65-71.
Trommer, H., et al. (2003) Int J Pharm, 254(2):223-34.
Tsuchida, K., et al. (1999) Eur. J. Biochem. 264, 461467.
Uebelhart, D., and Williams, J. M. (1999) Curr Opin Rheumatol, 11, 427-435.
Unligil, U. M. and J. M. Rini. (2000) Curr. Opin. Struct. Biol. 10, 510-517.
Unligil, U. M., et al. (2000) EMBO J. 19, 5269-5280.
van de Rijn, I. and R. E. Kessler. (1980) Infect Immun. 27, 444-448.
van de Rijn, I. and R. R. Drake (1992) J. Biol. Chem. 267, 24302-24306.
Van Etten, J. L., et al. (1985) Virology, 140:135.
Vann, W. F., et al. (1981) Eur. J. Biochem. 116,359-364.
Varki, A. (1996) Proc. Natl. Acad. Sci. U.S.A. 93, 4523-4525.
Vimr, E. R., et al. (1989) J. Bacteriol. 171, 1106-1117.
Vincent, T., et al. (2003) Br J Haematol, 121(2):259-69.
Vlodavsky, I et al. (1996) Cancer Metastasis, 15:177-186.
Vrielink, A., et al. (1994) EMBO J. 15, 3413-3422.
Ward, J. A., et al. (2003) Am J Pathol, 162(5):1403-9.
Weigel, P. H., et al. (1997) J. Biol. Chem., 272, 13997-14000.
Wessels, M. R., et al. (1991) Proc. Natl. Acad. Sci. U.S.A. 88, 8317-8321.
West, D. C., et al. (1985) Science, 228(4705):1324-6.
West, D. C., and Kumar, S. (1989) Ciba Found Symp, 143, 187-201.
West, D. C. and S. Kumar. (1989) Exp Cell Res, 183(1):179-96.
West, D. C. and S. Kumar. (1991) Int J Radiat Biol, 60(1-2):55-60.
West, D. C., et al. (1985) Science, 228:1324-1326.
Wiggins, C. A. R., and S. Munro. (1998) Proc. Natl. Acad. Sci. U.S.A. 95, 7945-7950.
Williams, J. M., et al. (2003) Osteoarthritis Cartilage, 11(1):44-9.
Wilson, K. Current Protocols in Molecular Biology. New York: Wiley Interscience Publishing, 1987: 2.4.1-2.4.5.
Wolf, D., et al. (2001) Hepatology, 34(3):535-47.
Woodfield, T B, et al. (2002) Crit Rev Eukaryot Gene Expr, 12:209-236.
Yamada, T., et al. (1991) Appl. Environ. Microbiol. 57:3433.
Yasuda, M., et al. (2002) Histol. Histopathol. 17(3):945-950.
Yoshida, M., et al. (2000) J. Biol. Chem., 275, 497-506.
Zeng, C., et al. (1998) Int J Cancer, 77(3):396-401.
Zimmerman, e., et al. (2002) Biophys J, 82(4):1848-57.

1. A method of inhibiting or preventing a disease or condition in a patient, comprising the steps of:
identifying a disease or condition in a patient;
selecting a glycosaminoglycan polymer having a specific size distribution, wherein the glycosaminoglycan polymer having the specific size distribution is effective in inhibiting the disease or condition;
providing a composition comprising recombinantly-produced defined glycosaminoglycan polymers having the desired specific size distribution such that the glycosaminoglycan polymers are substantially monodisperse in size, wherein at least 95% of the composition comprises the defined glycosaminoglycan polymers having the desired specific size distribution and less than 5% of the composition comprises glycosaminoglycan polymers of a different size distribution; and
administering to the patient an effective amount of the composition to inhibit the disease or condition.

2. The method of claim 1 wherein the substantially monodisperse glycosaminoglycan polymers have a molecular weight in a range of from about 600 Da to about 3.5 kDa.

3. The method of claim 2 wherein the substantially monodisperse glycosaminoglycan polymers have a polydispersity value in a range of from about 1.0 to about 1.1.

4. The method of claim 2 wherein the substantially monodisperse glycosaminoglycan polymers have a polydispersity value in a range of from about 1.0 to about 1.05.

5. The method of claim 1 wherein the defined glycosaminoglycan polymers are defined hyaluronan polymers having a size distribution in a range of from HA10 to HA25.

6. The method of claim 5 wherein the hyaluronan polymer is HA10.

7. The method of claim 5 wherein the hyaluronan polymer is HA12.

8. The method of claim 5 wherein the hyaluronan polymer is HA20.

9. The method of claim 5 wherein the hyaluronan polymer is HA22.

10. The method of claim 1 wherein the glycosaminoglycan polymers are chimeric or hybrid glycosaminoglycans having a non-natural structure.

11. The method of claim 1 wherein the disease or condition is cancer.

12. The method of claim 1 wherein the disease or condition is a disease or condition associated with abnormal levels of angiogenesis.

13. The method of claim 1 wherein a different size distribution of the glycosaminoglycan polymer is not effective in inhibiting the disease or condition.

14. The method of claim 1 wherein, the disease or condition is a first type of cancer, and the desired size distribution of the glycosaminoglycan polymer is effective in inhibiting the first type of cancer, but is not effective in inhibiting a second type of cancer.

15. The method of claim 1 wherein the defined glycosaminoglycan polymer is produced by a method comprising the steps of:
providing at least one functional acceptor, wherein the functional acceptor has at least two sugar units selected from the group consisting of uronic acid, hexosamine, structural variants and derivatives thereof, a hyaluronan polymer, a chondroitin polymer, a chondroitin sulfate polymer, a heparosan-like polymer, a heparinoid, mixed GAG chains, analog containing chains, and combinations thereof;
providing at least one recombinant glycosaminoglycan transferase capable of elongating the at least one functional acceptor in at least one of a controlled fashion and a repetitive fashion to form extended glycosaminoglycan-like molecules; and
providing at least one UDP-sugar selected from the group consisting of UDP-GlcUA, UDP-GlcNAc, UDP-Glc, UDP-GalNAc, UDP-GlcN, UDP-GalN and structural variants or derivatives thereof in a stoichiometric ratio to the at least one functional acceptor such that the at least one recombinant glycosaminoglycan transferase elongates the at least one functional acceptor to provide glycosaminoglycan polymers wherein the glycosaminoglycan polymers have a desired size distribution such that the glycosaminoglycan polymers are substantially monodisperse in size.

16. The method of claim 15 wherein, in the step of providing at least one functional acceptor, uronic acid is further defined as a uronic acid selected from the group consisting of GlcUA, IdoUA, GalUA, and structural variants or derivatives thereof, and hexosamine is further defined as a hexosamine selected from the group consisting of GlcNAc, GalNAc, GlcN, GalN, and structural variants or derivatives thereof.

17. The method of claim 15 wherein, in the step of providing at least one functional acceptor, the functional acceptor is selected from the group consisting of a chondroitin oligosaccharide comprising at least about three sugar units, a chondroitin polymer, a chondroitin sulfate polymer, a heparosan-like polymer, a heparinoid, and an extended acceptor selected from the group consisting of HA chains, chondroitin chains, heparosan chains, mixed glycosaminoglycan chains, analog containing chains, a sulfated functional acceptor, a modified oligosaccharide, and combinations thereof.

18. The method of claim 15 wherein, in the step of providing at least one recombinant glycosaminoglycan transferase, the at least one recombinant glycosaminoglycan transferase is selected from the group consisting of a recombinant hyaluronan synthase or active fragment or mutant thereof; a recombinant chondroitin synthase or active fragment or mutant thereof; a recombinant heparosan synthase or active fragment or mutant thereof; a recombinant single action glycosyltransferase capable of adding only one of GlcUA, GlcNAc, Glc, GalNAc, GlcN, GalN or a structural variant or derivative thereof; a recombinant synthetic chimeric glycosaminoglycan transferase capable of adding two or more of GlcUA, GlcNAc, Glc, GalNAc, GlcN, GalN or a structural variant or derivative thereof; and combinations thereof.

19. The method of claim 15 further comprising at least one of (A) through (D):
(A) the at least one functional acceptor is a plurality of functional acceptors immobilized on a substrate;
(B) the at least one functional acceptor is a plurality of functional acceptors in a liquid phase;
(C) the at least one recombinant glycosaminoglycan transferase is immobilized and the at least one functional acceptor and the at least one of UDP-GlcUA, UDP-GlcNAc, UDP-Glc, UDP-GalNAc, UDP-GlcN, UDP-GalN and a structural variant or derivative thereof are in a liquid phase; and
(D) the at least one functional acceptor is immobilized and the at least one UDP-sugar are in a liquid phase.

20. The method of claim 15, further comprising the step of providing a divalent metal ion, wherein the divalent metal ion is selected from the group consisting of manganese, magnesium, cobalt, nickel and combinations thereof.

21. The method of claim 15, wherein the method occurs in a buffer having a pH from about 6 to about 8.

22. The method of claim 15 wherein, in the step of providing the at least one recombinant glycosaminoglycan transferase, the at least one recombinant glycosaminoglycan transferase is selected from the group consisting of:
(A) a recombinant glycosaminoglycan transferase having an amino acid sequence encoded by a nucleotide sequence capable of hybridizing under standard stringent, moderately stringent, or less stringent hybridization conditions to a nucleotide sequence selected from the group consisting of SEQ ID NOS:1, 3, 5, 7, 9 or 11;
(B) a recombinant glycosaminoglycan transferase having an amino acid sequence essentially as set forth in SEQ ID NO:2, 4, 6, 8, 10, 12-22 or 25;
(C) a recombinant glycosaminoglycan transferase encoded by a nucleotide sequence essentially as set forth in SEQ ID NO:1, 3, 5, 7, 9 or 11; and
(D) a recombinant glycosaminoglycan transferase having at least one motif selected from the group consisting of SEQ ID NOS:23 and 24.

23. The method of claim 15 wherein, in the step of providing at least one functional acceptor, the at least one functional acceptor comprises a moiety selected from the group consisting of a fluorescent tag, a radioactive tag, an affinity tag, a detection probe, a medicant, and combinations thereof.

24. The method of claim 15 wherein, in the step of providing at least one UDP-sugar, at least one UDP-sugar is radioactively labeled.

25-47. (canceled)

48. A kit, comprising:
at least two compositions comprising recombinantly-produced defined glycosaminoglycan polymers having desired specific size distributions such that the glycosaminoglycan polymers of each composition are substantially monodisperse in size, wherein at least 95% of the compositions comprise the defined glycosaminoglycan polymers having the desired specific size distribution and less than 5% of the compositions comprise glycosaminoglycan polymers of a different size distribution, and wherein the at least two compositions comprise recombinantly-produced defined glycosaminoglycan polymers having different specific size distributions; and
means for testing the ability of each of the defined glycosaminoglycan polymers to inhibit or prevent a disease or condition in a sample from a patient.

49. The kit of claim 48 wherein the sample from the patient is a biopsy.

50. The kit of claim 48 wherein the disease or condition is cancer.

51. The kit of claim 48 wherein the disease or condition is a disease or condition associated with abnormal levels of angiogenesis.

52. The kit of claim 48 wherein one desired size distribution of the glycosaminoglycan polymer is effective in inhibiting or preventing the disease or condition, while a different size distribution of the glycosaminoglycan polymer is not effective in inhibiting or preventing the disease or condition.

53. The kit of claim 48 wherein the kit is a catalog available on the World Wide Web.

54. The kit of claim 48 wherein each of the at least two substantially monodisperse glycosaminoglycan polymers have a molecular weight in a range of from about 600 Da to about 3.5 kDa.

55. The kit of claim 54 wherein the substantially monodisperse glycosaminoglycan polymers have a polydispersity value in a range of from about 1.0 to about 1.1.

56. The kit of claim 54 wherein the substantially monodisperse glycosaminoglycan polymers have a polydispersity value in a range of from about 1.0 to about 1.05.

57. The kit of claim 48 wherein the at least two glycosaminoglycan polymers are hyaluronan polymers having a size distribution in a range of from HA10 to HA25.

58. The kit of claim 57 wherein one of the at least two hyaluronan polymers is HA10.

59. The kit of claim 57 wherein one of the at least two hyaluronan polymers is HA12.

60. The kit of claim 57 wherein one of the at least two hyaluronan polymers is HA20.

61. The kit of claim 57 wherein one of the at least two hyaluronan polymers is HA22.

62-71. (canceled)

72. A method of inhibiting or preventing a disease or condition in a patient, comprising the steps of:
providing at least two compositions comprising recombinantly-produced defined glycosaminoglycan polymers having desired specific size distributions such that the glycosaminoglycan polymers of each composition are substantially monodisperse in size, wherein at least 95% of the compositions comprise the defined glycosaminoglycan polymers having the desired specific size distribution and less than 5% of the compositions comprise glycosaminoglycan polymers of a different size distribution, and wherein the at least two compositions comprise recombinantly-produced defined glycosaminoglycan polymers having different specific size distributions;
providing a sample from a patient suffering from or predisposed for a disease or condition;
reacting each of the at least two defined glycosaminoglycan polymer compositions with a portion of the sample from the patient;
identifying at least one defined glycosaminoglycan polymer composition that inhibits or prevents the disease or condition in the sample; and
administering to the patient an effective amount of the defined glycosaminoglycan polymer composition that inhibited or prevented the disease or condition in the sample, thus inhibiting or preventing the disease or condition in the patient.

73. The method of claim 72 wherein the sample from the patient is a biopsy.

74. The method of claim 72 wherein the disease or condition is cancer.

75. The method of claim 72 wherein the disease or condition is a disease or condition associated with abnormal levels of angiogenesis.

76. The method of claim 72 wherein one desired size distribution of the glycosaminoglycan polymer is effective in inhibiting or preventing the disease or condition, while a different size distribution of the glycosaminoglycan polymer is not effective in inhibiting or preventing the disease or condition.

77. The method of claim 72 wherein each of the at least two substantially monodisperse glycosaminoglycan polymers have a molecular weight in a range of from about 600 Da to about 3.5 kDa.

78. The method of claim 77 wherein the substantially monodisperse glycosaminoglycan polymers have a polydispersity value in a range of from about 1.0 to about 1.1.

79. The method of claim 77 wherein the substantially monodisperse glycosaminoglycan polymers have a polydispersity value in a range of from about 1.0 to about 1.05.

80. The method of claim 72 wherein the at least two glycosaminoglycan polymers are hyaluronan polymers having a size distribution in a range of from HA10 to HA25.

81. The method of claim 80 wherein one of the at least two hyaluronan polymers is HA10.

82. The method of claim 80 wherein one of the at least two hyaluronan polymers is HA12.

83. The method of claim 80 wherein one of the at least two hyaluronan polymers is HA20.

84. The method of claim 80 wherein one of the at least two hyaluronan polymers is HA22.

85-93. (canceled)


SEQUENCE LISTING (25 sequences)

SEQ ID NO: 1 - DNA, 2920 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatattaaaatcatccagaataaagatgccaaaatcgcagtcagtattttttatcccaatacattaaacggcttagtgaaaaaactaaacaatattattgaatataataaaaatatattcgttattgttctacatgttgataagaatcatcttacaccagatatcaaaaaagaaatactagccttctatcataaacatcaagtgaatattttactaaataatgatatctcatattacacgagtaatagattaataaaaactgaggcgcatttaagtaatattaataaattaagtcagttaaatctaaattgtgaatacatcatttttgataatcatgacagcctattcgttaaaaatgacagctatgcttatatgaaaaaatatgatgtcggcatgaatttctcagcattaacacatgattggatcgagaaaatcaatgcgcatccaccatttaaaaagctcattaaaacttattttaatgacaatgacttaaaaagtatgaatgtgaaaggggcatcacaaggtatgtttatgacgtatgcgctagcgcatgagcttctgacgattattaaagaagtcatcacatcttgccagtcaattgatagtgtgccagaatataacactgaggatatttggttccaatttgcacttttaatcttagaaaagaaaaccggccatgtatttaataaaacatcgaccctgacttatatgccttgggaacgaaaattacaatggacaaatgaacaaattgaaagtgcaaaaagaggagaaaatatacctgttaacaagttcattattaatagtataactctataaa

SEQ ID NO: 2 - protein, 972 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 3 - DNA, 2979 bp, Pasteurella multocida
ttataaactgattaaagaaggtaaacgattcaagcaaggttaatttttaaaggaaagaaaatgaatacattatcacaagcaataaaagcatataacagcaatgactatgaattagcactcaaattatttgagaagtctgctgaaacctacgggcgaaaaatcgttgaattccaaattatcaaatgtaaagaaaaactctcgaccaattcttatgtaagtgaagataaaaaaaacagtgtttgcgatagctcattagatatcgcaacacagctcttactttccaacgtaaaaaaattaactctatccgaatcagaaaaaaacagtttaaaaaataaatggaaatctatcactgggaaaaaatcggagaacgcagaaatcagaaaggtggaactagtacccaaagattttcctaaagatcttgttcttgctccattgccagatcatgttaatgattttacatggtacaaaaatcgaaaaaaaagcttaggtataaagcctgtaaataagaatatcggtctttctattattattcctacatttaatcgtagccgtattttagatataacgttagcctgtttggtcaatcagaaaacaaactacccatttgaagtcgttgttgcagatgatggtagtaaggaaaacttacttaccattgtgcaaaaatacgaacaaaaacttgacataaagtatgtaagacaaaaagattatggatatcaattgtgtgcagtcagaaacttaggtttacgtacagcaaagtatgattttgtctcgattctagactgcgatatggcaccacaacaattatgggttcattcttatcttacagaactattagaagacaatgatattgttttaattggacctagaaaatatgtggatactcataatattaccgcagaacaattccttaacgatccatatttaatagaatcactacctgaaaccgctacaaataacaatccttcgattacatcaaaaggaaatatatcgttggattggagattagaacatttcaaaaaaaccgataatctacgtctatgtgattctccgtttcgttattttagttgcggtaatgttgcattttctaaagaatggctaaataaagtaggttggttcgatgaagaatttaatcattgggggggcgaagatgtagaatttggttacagattatttgccaaaggctgttttttcagagtaattgacggcggaatggcataccatcaagaaccacctggtaaagaaaatgaaacagaccgcgaagctggtaaaagtattacgcttaaaattgtgaaagaaaaggtaccttacatctatagaaagcttttaccaatagaagattcacatattcatagaatacctttagtttctatttatatccccgcttataactgtgcaaattatattcaaagatgtgtagatagtgctcttaatcaaactgttgtcgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccattttagaatgtttacgattagagcttggcatttaacggatggatttaacgaaaatattgaaaacgccgtggattatgacatgttccttaaactcagtgaagttggaaaatttaaacatcttaataaaatctgctataaccgcgtattacatggtgataacacatccattaagaaactcggcattcaaaagaaaaaccattttgttgtagtcaatcagtcattaaatagacaaggcatcaattattataattatgacaaatttgatgatttagatgaaagtagaaagtatatcttcaataaaaccgctgaatatcaagaagaaatggatattttaaaagatcttaaactcattcaaaataaagatgccaaaatcgcagtcagtattttctatcccaatacattaaacggcttagtgaaaaaactaaacaatattattgaatataataaaaatatattcgttattattctacatgttgataagaatcatcttacaccagacatcaaaaaagaaatattggctttctatcataagcaccaagtgaatattttactaaataatgacatctcatattacacgagtaatagactaataaaaactgaggcacatttaagtaatattaataaattaagtcagttaaatctaaattgtgaatacatcatttttgataatcatgacagcctattcgttaaaaatgacagctatgcttatatgaaaaaatatgatgtcggcatgaatttctcagcattaacacatgattggatcgagaaaatcaatgcgcatccaccatttaaaaagctgattaaaacctattttaatgacaatgacttaagaagtatgaatgtgaaaggggcatcacaaggtatgtttatgaagtatgcgctaccgcatgagcttctgacgattattaaagaagtcatcacatcctgccaatcaattgatagtgtgccagaatataacactgaggatatttggttccaatttgcacttttaatcttagaaaagaaaaccggccatgtatttaataaaacatcgaccctgacttatatgccttgggaacgaaaattacaatggacaaatgaacaaattcaaagtgcaaaaaaaggcgaaaatatccccgttaacaagttcattattaatagtataacgctataaaacatttgcattttattaaaa

SEQ ID NO: 4 - protein, 965 aa, Pasteurella multocida
MNTLSQAIKAYNSNDYELALKLFEKSAETYGRKIVEFQIIKCKEKLSTNSYVSEDKKNSVCDSSLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNENIENAVDYDMFLKLSEVGKFKHLNKICYNRVLHGDNTSIKKLGIQKKNHFVVVNQSLNRQGINYYNYDKFDDLDESRKYIFNKTAEYQEEMDILKDLKLIQNKDAKIAVSIFYPNTLNGLVKKLNNIIEYNKNIFVIILHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLRSMNVKGASQGMFMKYALPHELLTIIKEVITSCQSIDSVPEYNTEDIWFQFALLILEKKTGHVFNKTSTLTYMPWERKLQWTNEQIQSAKKGENIPVNKFIINSITL

SEQ ID NO: 5 - DNA, 1851 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacacaaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccactattaaataataatgctaattattctgaacttattccgtgtttatctttttataacgag

SEQ ID NO: 6 - protein, 615 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNLEVIVVDDYSTDKTFQIASRIANSTSKVKTFRLNSNLGTYFAKNTGILKSKGDIIFFQSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYTNTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 7 - DNA, 1854 bp, Pasteurella multocida
atgagcttatttaaacgtgctactgagctatttaagtcaggaaactataaagatgcactaactctatatgaaaatatagctaaaatttatggttcagaaagccttgttaaatataatattgatatatgtaaaaaaaatataacacaatcaaaaagtaataaaatagaagaagataatatttctggagaaaacaaattttcagtatcaataaaagatctatataacgaaataagcaatagtgaattagggattacaaaagaaagactaggagccccccctctagtcagtattataatgacttctcataatacagaaaaattcattgaagcctcaattaattcactattattgcaaacatacaataacttagaagttatcgttgtagatgattatagcacagataaaacatttcagatcgcatccagaatagcaaactctacaagtaaagtaaaaacattccgattaaactcaaatctagggacatactttgcgaaaaatacaggaattttaaagtctaaaggagatattattttctttcaggatagcgatgatgtatgtcaccatgaaagaatcgaaagatgtgttaatgcattattatcgaataaagataatatagctgttagatgtgcatattctagaataaatctagaaacacaaaatataataaaagttaatgataataaatacaaattaggattaataactttaggcgtttatagaaaagtatttaatgaaattggtttttttaactgcacaaccaaagcatcggatgatgaattttatcatagaataattaaatactatggtaaaaataggataaataacttatttctaccactgtattataacacaatgcgtgaagattcattattttctgatatggttgagtgggtagatgaaaataatataaagcaaaaaacctctgatgctagacaaaattatctccatgaattccaaaaaatacacaatgaaaggaaattaaatgaattaaaagagatttttagctttcctagaattcatgacgccttacctatatcaaaagaaatgagtaagctcagcaaccctaaaattcctgtttatataaatatatgctcaataccttcaagaataaaacaacttcaatacactattggagtactaaaaaaccaatgcgatcattttcatatttatcttgatggatatccagaagtacctgattttataaaaaaactagggaataaagcgaccgttattaattgtcaaaacaaaaatgagtctattagagataatggaaagtttattctattagaaaaacttataaaggaaaataaagatggatattatataacttgtgatgatgatatccggtatcctgctgactacataaacactatgataaaaaaaattaataaatacaatgataaagcagcaattggattacatggtgttatattcccaagtagagtcaacaagtatttttcatcagacagaattgtctataattttcaaaaacctttagaaaatgatactgctgtaaatatattaggaactggaactgttgcctttagagtatctatttttaataaattttctctatctgattttgagcatcctggcatggtagatatctatttttctatactatgtaagaaaaacaatatactccaagtttgtatatcacgaccatcgaattggctaacagaagataacaaaaacactgagaccttatttcatgaattccaaaatagagatgaaatacaaagtaaactcattatttcaaacaacccttggggatactcaagtatatatccattattaaataataatgctaattattctgaacttattccgtgtttatctttttataacgagtaa

SEQ ID NO: 8 - protein, 617 aa, Pasteurella multocida
MSLFKRATELFKSGNYKDALTLYENIAKIYGSESLVKYNIDICKKNITQSKSNKIEEDNISGENKFSVSIKDLYNEISNSELGITKERLGAPPLVSIIMTSHNTEKFIEASINSLLLQTYNNLEVIVVDDYSTDKTFQIASRIANSTSKVKTFRLNSNLGTYFAKNTGILKSKGDIIFFQDSDDVCHHERIERCVNALLSNKDNIAVRCAYSRINLETQNIIKVNDNKYKLGLITLGVYRKVFNEIGFFNCTTKASDDEFYHRIIKYYGKNRINNLFLPLYYNTMREDSLFSDMVEWVDENNIKQKTSDARQNYLHEFQKIHNERKLNELKEIFSFPRIHDALPISKEMSKLSNPKIPVYINICSIPSRIKQLQYTIGVLKNQCDHFHIYLDGYPEVPDFIKKLGNKATVINCQNKNESIRDNGKFILLEKLIKENKDGYYITCDDDIRYPADYINTMIKKINKYNDKAAIGLHGVIFPSRVNKYFSSDRIVYNFQKPLENDTAVNILGTGTVAFRVSIFNKFSLSDFEHPGMVDIYFSILCKKNNILQVCISRPSNWLTEDNKNTETLFHEFQNRDEIQSKLIISNNPWGYSSIYPLLNNNANYSELIPCLSFYNE

SEQ ID NO: 9 - DNA, 780 bp, Pasteurella multocida
aacaggggataaggtcagtaaatttaggatgatttttgactaatggataaatacttgaatatccccatggaccgttttccatgatcagctgagtttgttgctcatcattgtctcgatattgatgatagagtgtttcgctgtctctattatcttccgttagccagtttgctggtcttgaaatacaaatctgaagaatattatttttcttacacaagagagagaaatagatatcagccatgcctgaatgggtaaagtcagaaagagaaaattgattaaagagactgactctaaagctaacagttcctgtacctaatacattgaccgctttgtctttttccagaggtttatagaagctatataccagtctatccgccgaaaaatatttggtcattctacttggaaagagaatgccgtgtaaaccaataaccgctttatcatcgtattcattcagcttcttgatcatcgtattgatgtaatcgcttggatagataatgtcatcatcacaggttatataatatccatcttgatttttttcaatcaactcttccagtaaaatgaatttgccattatctctaatggagttatctttatctttgcaatgaacaacggttgctttattacctaaattttttatgaagtcagggatttctacatagccatcaagataaatatgaaaatgatcacattgattttttagtatgccgataatacgtcgtaattgcgctattcttgagggaatagaacaaatattgatataaacaggaatcttaggattggaca

SEQ ID NO: 10 - protein, 651 aa, Pasteurella multocida
MKRKKEMTQKQMTKNPPQHEKENELNTFQNKIDSLKTTLNKDIISQQTLLAKQDSKHPLSASLENENKLLLKQLQLVLQEFEKIYTYNQALEAKLEKDKQTTSITDLYNEVAKSDLGLVKETNSVNPLVSIIMTSHNTAQFIEASINSLLLQTYKNIEIIIVDDDSSDNTFEIASRIANTTSKVRVFRLNSNLGTYFAKNTGILKSKGDIIFFQDSDDVCHHERIERCVNILLANKETIAVRCAYSRLAPETQHIIKVNNMDYRLGFITLGMHRKVFQEIGFFNCTTKGSDDEFFHRIAKYYGKEKIKNLLLPLYYNTMRENSLFTDMVEWIDNHNIIQKMSDTRQHYATLFQAMHNETASHDFKNLFQFPRIYDALPVPQEMSKLSNPKIPVYINICSIPSRIAQLRRIIGILKNQCDHFHIYLDGYVEIPDFIKNLGNKATVVHCKDKDNSIRDNGKFILLEELIEKNQDGYYITCDDDIIYPSDYINTMIKKLNEYDDKAVIGLHGILFPSRMTKYFSADRLVYSFYKPLEKDKAVNVLGTGTVSFRVSLFNQFSLSDFTHSGMADIYFSLLCKKNNILQICISRPANWLTEDNRDSETLYHQYRDNDEQQTQLIMENGPWGYSSIYPLVKNHPKFTDLIPCLPFYFL

SEQ ID NO: 11 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 12 - protein, 703 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDI

SEQ ID NO: 13 - DNA, 1980 bp, Pasteurella multocida
atgctctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 14 - DNA, 1902 bp, Pasteurella multocida
atgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 15 - DNA, 1830 bp, Pasteurella multocida
atgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 16 - DNA, 1764 bp, Pasteurella multocida
atgcttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 17 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcaactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 18 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtaatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 19 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttaaattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 20 - DNA, 2112 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgcaaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcaaatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatatttaa

SEQ ID NO: 21 - protein, 703 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLNCNMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDI

SEQ ID NO: 22 - protein, 703 aa, Pasteurella multocida
MNTLSQAIKAYNSNDYQLALKLFEKSAEIYGRKIVEFQITKCKEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLNSNDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDI

SEQ ID NO: 23 - protein, 76 aa, Artificial Sequence
  motif
QTYXNXEXXXXDDXXXDXXXXXXXXIAXXXXXVXXXXXXXNXGXYXXXXXXXXXXXXXXXXFQDXDDXXHXERIXR

SEQ ID NO: 24 - protein, 102 aa, Artificial Sequence
  motif
XDXGKFIXXXXXXXXXXXXXXXXXDDDIXYPXDYXXXMXXXXXXXXXXXXXXXXXXXXXXXXXXXXXXXXXXXXXXXXXXXXXXXXXXXXXXXVNXLGTGTV

SEQ ID NO: 25 - protein, 704 aa, Pasteurella multocida
MNTLSQAIKAYNSNDYELALKLFEKSAETYGRKIVEFQIIKCKEKLSTNSYVSEDKKNSVCDSSLDIATQLLLSNVKKLTLSESEKNSLKNKWKSITGKKSENAEIRKVELVPKDFPKDLVLAPLPDHVNDFTWYKNRKKSLGIKPVNKNIGLSIIIPTFNRSRILDITLACLVNQKTNYPFEVVVADDGSKENLLTIVQKYEQKLDIKYVRQKDYGYQLCAVRNLGLRTAKYDFVSILDCDMAPQQLWVHSYLTELLEDNDIVLIGPRKYVDTHNITAEQFLNDPYLIESLPETATNNNPSITSKGNISLDWRLEHFKKTDNLRLCDSPFRYFSCGNVAFSKEWLNKVGWFDEEFNHWGGEDVEFGYRLFAKGCFFRVIDGGMAYHQEPPGKENETDREAGKSITLKIVKEKVPYIYRKLLPIEDSHIHRIPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNENIENAVDYDMFLKLSEVGKFKHLNKICYNRVLHGDNTSIKKLGIQKKNHFVVVNQSLNRQGINYYNYDKFDDLDESRKYIFNKTAEYQEEMDILKDLKLIQNKDA